(19) United States
(12) Patent Application Publication
(10) Pub. No.: US 2015/0051352 A1
(43) Pub. Date: Feb. 19, 2015

(54) MULTI-FUNCTIONAL GRAFTED POLYMERS

(71) Applicant: ISP Investments Inc., Wilmington, DE (US)

(72) Inventors: Osama M. Musa, Kinnelon, NJ (US); Xuejun Liu, Whippany, NJ (US); Jui-Chang Chuang, Wayne, NJ (US); Karen Winkowski, Springfield, NJ (US)

(21) Appl. No.: 14/384,090

(22) PCT Filed: Mar. 11, 2013
(86) PCT No.: PCT/US13/30115

§ 371 Date: Sep. 9, 2014

Related U.S. Application Data
(60) Provisional application No. 61/608,962, filed on Mar. 9, 2012.

(51) Int. Cl. C08F 26/06; A61Q 19/00; A61K 8/81

(52) U.S. Cl. CPC C08F 26/06; A61K 8/8164; A61Q 19/00; A61K 2800/74
USPC 525/326.7

(57) ABSTRACT

Provided are multifunctional polymers comprising at least one anhydride repeating unit with at least one pseudo-cationic moiety graft and at least one hydrophobic graft. The grafting functionalizations can occur before, during, or after polymerizing the monomer(s). The anhydride employed may be partially or fully opened to provide amic acids, carboxylic acids, carboxylic acidic salts, imides, esters, and mixtures thereof. The polymers also may comprise other repeating units, including, but not limited to, alpha-olefins. In one embodiment the polymers exhibit antimicrobial activity, and can be employed in a wide variety of compositions, including those where antimicrobial activity is useful. A method of providing microbial activity also is provided.

### BACKGROUND

1. Field of the Invention

The invention provides multifunctional polymers. The polymers may be prepared by functionalization of copolymers of an alkene and a maleic anhydride or copolymers of a vinyl ether and a maleic anhydride with a hydrophilic functionality and a hydrophobic functionality. The maleic anhydride employed may be partially or fully opened to provide amic acids, carboxylic acids, carboxylic acidic salts, imides, esters, and mixtures thereof. The polymers exhibit broad spectrum antimicrobial activity, useful skin/hair care properties and are compatible with cosmetic ingredients. The multifunctional polymers of the invention can be employed in a wide variety of compositions.

2. Description of Related Art

Antimicrobial compounds are widely used in many formulations, where they may assist in killing or inhibiting the growth and presence of microbes as bacterium, fungus, or protozoan, or combinations thereof. In the personal care arts antimicrobial compounds may be called “preservatives,” while in non-personal care applications—such as adhesives, coatings, inks, membranes, textiles, and paints—antimicrobial compounds may be called “biocides.” Regardless, regulatory and environmental concerns have put limits on the selection and usage of traditional preservatives, such as formaldehyde-donors, parabens, iodo-2-propynylbutylcarbamate (IPBC), and other active ingredients. Non-traditional preservatives, such as multifunctional polymers, have attracted much attention in the chemical industry. Antimicrobial polymers are nonvolatile, do not penetrate the skin, have better long-term efficiency and possibly higher selectivity compared to traditional preservatives. Antimicrobial polymers also minimize environmental problems by minimizing residual toxicity.

Multifunctional polymers are described in the following disclosures, each of which is incorporated herein by reference. De Grado, et al., in J. Am. Chem. Soc., 2005, 127, 4128, and U.S. Pat. Appl. No. 2006/0024264 disclose the synthesis and uses of amphiphilic polymethacrylate derivatives as antimicrobial agents. Kuroda, et al., in Chem. Eur. J., 2009, 15, 1123, describes the role of hydrophobicity in the antimicrobial and hemolytic activities of polymethacrylate derivatives. Gellman, et al., in Org. Lett., 2004, 4, 557, discloses the biocidal activity of polystyrene derivatives bearing cationic properties through reversible amine protonation. U.S. Pat. No. 6,214,885 describes the use of polymers containing β-hydroxyalkylvinylamine units as biocides. U.S. Pat. No. 5,208,016 discloses antimicrobial resin compositions containing ethylene copolymer from radical polymerization of ethylene and dialkylaminoalkylacrylamide comonomers.

Other references related to these polymers include the following patents and patent applications: EP 40,498; GB 686,381; 730,463; 870,398; 922,878; 1,286,966; 1,329,033; JP 53,090,397; 57,161,859; U.S. Pat. Nos. 3,449,250; 3,555,001; 4,048,422; 4,058,491; 4,734,446; 4,767,616; 5,229,458; 5,352,729; 5,408,022; 5,449,775; 5,492,988; 5,756,181; 6,025,501; 6,071,993; 6,075,107; 6,299,866; 6,646,082; 6,682,725; 6,737,049; 6,838,078; 6,951,598; 7,033,607; 7,041,281; 7,323,163; 7,326,262; 7,592,040; 7,955,594; US 2005/0152855; 2006/0024264; 2007/0082196; 2007/0161519; 2007/0238807; 2009/0029129; 2009/0312214; 2010/00029838; 2010/00298504; 2010/0130678; 2010/0137455; 2010/0174040; 2011/0060166, and WO 2010/0014655; 2010/031144.

Accordingly, there is a need for multifunctional polymers to alter or improve the physicochemical properties of such polymers.

### SUMMARY

Functionalized polymers has been discovered that in part exhibit antimicrobial activity. The polymers comprise at least one anhydride repeating unit with at least one pseudo-cationic moiety graft and at least one hydrophobic graft. The polymer may be a homopolymer of the anhydride-containing monomer, or may be a non-homopolymer when that monomer is polymerized with other monomers. The grafting functionalizations can occur before, during, or after polymerizing the monomer(s).

Given the options available to functionalize the polymers, they may be formulated into any number of compositions that may benefit from the polymers' antimicrobial activity. These compositions may or may not include other antibacterial compounds.

Also provided is a method of providing antimicrobial activity through the use of the multifunctional polymers.

### DETAILED DESCRIPTION

Multifunctional polymers are described that can exhibit antimicrobial and other properties valuable to many end-use applications. The polymers exhibit broad spectrum antimicrobial activity against gram-positive and gram-negative bacteria, and may be synthesized from at least two routes. In various embodiments, the weight-average molecular weight of the polymers ranges from about 1,000 to about 50,000 Da. They may be employed singly in these application, or may be formulated with or without other ingredients, including other multifunctional polymers, preservatives, and/or biocides, as necessary. Non-limiting examples of compositions having one or more multifunctional polymers include: adhesive, agricultural composition, beverage composition, coating composition, pharmaceutical composition, nutrition composition, household/industrial/institutional composition, oilfield composition, personal care composition, pharmaceutical composition, or pigment composition.

As used herein, the following terms have the meanings set out below.

The term “microbe” refers to any bacterium, fungus, protozoan, and any combinations thereof.

The term “antimicrobial” refers to a substance that kills or inhibits the growth of microbes such as bacterium, fungus, or protozoan, or combinations thereof. Antimicrobials may kill microbes (microbiocidal) and/or prevent the growth of microbes (microbiostatic). The term “antimicrobial activity” refers to activity that kills and/or inhibits the growth of microbes.

The term “functionalized” refers to replacing one or more hydrogens with one or more non-hydrogen groups, for e.g., alkyl, alkoxy, cycloalkyl, alkenyl, cycloalkenyl, alkynyl, and/or aryl groups. Alkyl, alkenyl and/or alkynyl groups include C₁-C₆₀, more particularly C₁-C₃₆, and most particularly C₁-C₁₈ groups. Cycloalkyl groups include cyclopentane, cyclohexane, cycloheptane, and the like. Alkoxy groups include methoxy, ethoxy, n-propoxy, isopropoxy, and the like. Aryl groups include benzenes, naphthalenes (2 rings), anthracenes (3 rings), and the like.

The term “anion” refers to an ion with more electrons than protons, giving it a net negative charge.

The term “cation” refers to an ion with fewer electrons than protons, giving it a net positive charge.

The term “halogenated” refers to functionalizations involving chloro, bromo, iodo and fluoro. In one embodiment halogen may be bromo and/or chloro.

The term “branched and unbranched alkyl groups” refers to alkyl groups which may be straight chained or branched. The alkyl group may have from 1 to about 18 carbon atoms, more particularly, from 1 to about 10 carbon atoms, and yet more particularly from 1 to about 6 carbon atoms. Branched groups include iso-propyl, tert-butyl, sec-butyl, and the like.

The term “hydrocarbyl” refers to straight-chain and/or branched-chain groups comprising carbon and hydrogen atoms with optional heteroatom(s). Particularly, the hydrocarbyl group includes C₁-C₆₀, more particularly C₁-C₃₆, and most particularly C₁-C₁₈ alkyl and alkenyl groups optionally having one or more hetero atoms. The hydrocarbyl group may be mono-, di- or polyvalent.

The term “heteroatom” refers to oxygen, nitrogen, sulfur, silicon, and/or phosphorous. The heteroatom may be present as a part of one or more functional groups on the hydrocarbyl chain and/or as a part of the hydrocarbyl chain itself. When the heteroatom is a nitrogen atom, the nitrogen atom may be present in the form of a quaternary amine.

The term “generic substituent(s)” refer(s) to substituent(s) such as R₁-R₆, and integers x, y, and z used and defined in the invention.

The term “amphiphilic” refers to a compound possessing both hydrophilic (water-loving, polar) and hydrophobic (lipophilic, fat-loving, non-polar) properties. Such compounds are also referred to as amphipathic.

The term “C₁-C₂₀ alkyl” refers to groups such as: methyl, ethyl, n-propyl, iso-propyl, n-butyl, iso-butyl, sec-butyl, tert-butyl, n-pentyl, sec-pentyl, tert-pentyl, n-hexyl, n-heptyl, n-octyl, 2-ethylhexyl, n-nonyl, iso-nonyl, 2-propylheptyl, n-decyl, n-dodecyl, n-tridecyl, iso-tri-decyl, n-tetradecyl, n-hexydecyl, n-octadecyl and eicosyl.

The term “C₁-C₂₀ alkylene” refers to groups such as: methylene, ethylene, n-propylene, iso-propylene, n-butylene, iso-butylene, sec-butylene, tert-butylene, n-pentylene, sec-pentylene, tert-pentylene, n-hexylene, n-heptylene, n-octylene, 2-ethylhexylene, n-nonylene, iso-nonylene, 2-propylheptylene, n-decylene, n-dodecylene, n-tridecylene, iso-tri-decylene, n-tetradecylene, n-hexydecylene, n-octadecylene and eicosylene.

The term “pseudo-cationic moiety” refers to moiety that comprises one or more functionalized and unfunctionalized nitrogen or phosphorus.

The term “monomer” refers to a small molecule that chemically bonds during polymerization to one or more monomers of the same or different kind to form a polymer.

The term “polymer” refers to a large molecule (macromolecule) comprising repeating structural units polymerized from one or more monomers connected by covalent chemical bonds.

The term “polymerization” refers to methods for chemically reacting monomers to form polymer chains. The type of polymerization method may be selected from a wide variety of methods. Such methods include, but are not limited to, free radical polymerization, such as classical radical polymerization and controlled radical polymerization, Nitroxide Mediation Polymerization (NMP), Atom Transfer Radical Polymerization (ATRP), and Reversible Addition Fragmentation Chain-Transfer (RAFT).

The term “homopolymer” refers to a polymer comprising essentially one type of monomer. Homopolymers include polymers polymerized from one monomer that may be modified during or after polymerization, for example, by grafting, hydrolyzing, or end-capping. Homopolymers may be associated with solvent adducts.

The term “non-homopolymer” refers to a polymer obtained by polymerization of two or more different kinds of monomers. The definition includes essentially all polymers that are not homopolymers. Nonlimiting examples of non-homopolymers include copolymers, terpolymers, tetrapolymers, and the like, wherein the non-homopolymer may be a random, block, or an alternating polymer.

The term “hydrophilic” refers to a molecular entity that tends to be polar and water-soluble or water-miscible. A hydrophilic molecule or portion of a molecule may be charge-polarized and/or capable of hydrogen bonding enabling it to dissolve in water.

The term “hydrophobic” refers to a molecular entity that tends to be non-polar and non-water-soluble.

The term “inert solvent” refers to a solvent that does not interfere chemically with the reaction.

The term “lower molecular weight alcohols” refers to alcohols having from one to four carbon atoms. Examples of lower molecular weight alcohols include: methanol, ethanol, 1-propanol, 2-propanol, allyl alcohol, propargyl alcohol, 2-aminoethanol, ethylene glycol, methyl propargyl alcohol, 1-butyn-4-ol, 2-butyn-1-ol, 2-buten-1-ol, 2-butanol, 2-methyl-2-propanol, and tert-butanol. In various embodiments of the invention, the lower molecular weight alcohol may be methanol, ethanol, 1-propanol, 2-propanol, or tert-butanol, or combinations thereof.

The term “quaternary ammonium cation”, also known as “quat,” refers to a positively charged polyatomic ion having the structure NR′₄⁺, wherein each of the four R′ can independently be an alkyl group or an aryl group. Unlike the ammonium ion (NH₄⁺) and primary, secondary, and tertiary ammonium cations, the quaternary ammonium cations are permanently charged, independent of the pH value of their solution. Accordingly, quaternary ammonium cations are accompanied by an anion (negative charge) to balance the overall charge.

The term “are each independently selected from the group consisting of” means that when a group appears more than once in a structure, that group may be independently selected each time it appears. For example, in the structure below:

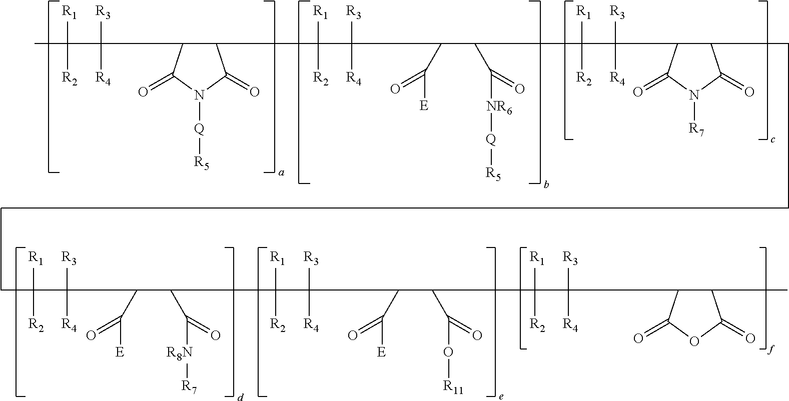

the generic substituents R₁-R₅, R₇, and Q and E each appear more than once. The term “are each independently selected from the group consisting of” means that each generic substituent may be the same or different.

The term “weight-average molecular weight” refers to a method of describing the molecular weight of a polymer, and may be calculated by the equation:

$${M}_{w} = \frac{{\sum }_{i} {N}_{i} {M}_{i}^{2}}{{\sum }_{i} {N}_{i} {M}_{i}}$$

wherein N₁ is the number of molecules having molecular weight Mᵢ.

The term “number-average molecular weight” refers to another method of describing the molecular weight of a polymer, and may be calculated by the equation:

$${M}_{n} = \frac{{\sum }_{i} {N}_{i} {M}_{i}}{{\sum }_{i} {N}_{i}}$$

wherein N₁ is the number of molecules having molecular weight Mᵢ.

The term “personal care composition,” also referred to as “cosmetics,” refer to such illustrative non-limiting compositions as skin, sun, oil, hair, and preservative compositions, including those to alter the color, condition, or appearance of the skin. Potential personal care compositions include, but are not limited to, compositions for increased flexibility in styling, durable styling, increased humidity resistance for hair, skin, color cosmetics, water-proof/resistance, wear-resistance, and thermal protecting/enhancing compositions.

The term “performance chemicals composition” refers to any non-personal care composition. Performance chemicals compositions serve a broad spectrum of arts, and include non-limiting compositions such as: adhesives; agricultural, biocides, coatings, electronics, household-industrial-institutional (HI&I), inks, membranes, metal fluids, oilfield, paper, paints, plastics, printing, plasters, and wood-care compositions.

The term “imide” refers to an organic compound comprising two carbonyl groups (acyl groups) bound to a common nitrogen atom. The nitrogen atom in the imide functional group may or may not be substituted with an organic functional group.

The term “Jeffamine” is a brand name of The Huntsman Corporate and refers to polyetheramines containing primary amino groups attached to the end of a polyether backbone. The polyether may be based on either propylene oxide (PO), ethylene oxide (EO), or mixed PO/EO. The polyetheramines undergo typical amine reactions, often imparting increased flexibility, toughness, low viscosity, and low color. The wide range of molecular weight, amine functionality, repeating unit type, and distribution can provide flexibility in the design of new compounds or mixtures. Jeffamines are available from Huntsman Corporation, The Woodlands, Tex.

Multifunctional polymers have been discovered that comprise at least: (A) at least a first repeating unit selected from the group consisting of:

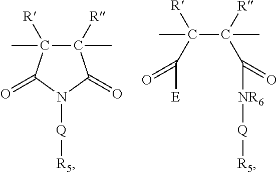

and combinations thereof, and (B) at least a second repeating unit is selected from the group consisting of:

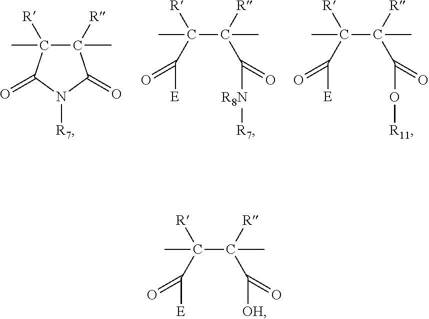

and combinations thereof, wherein each C— indicates a bond from said unit to another unit along the polymer backbone;
each R′ and R″ is independently selected from the group consisting of: hydrogen, alkyl, cycloalkyl, aryl, and combinations thereof;
each R₅ is independently selected from the group consisting of —NR₉R₁₀, functionalized and unfunctionalized nitrogen or phosphorus containing C₅-C₇ cyclic groups, and mixtures thereof;
each R₆, R₈, R₉, and R₁₀ is independently selected from the group consisting of hydrogen, functionalized and unfunctionalized alkyl, alkoxy, cycloalkyl, alkenyl, cycloalkenyl, aryl groups, wherein any of the before mentioned groups may be with or without heteroatoms, and mixtures thereof;
each R₇ and R₁₁ is independently selected from the group consisting of functionalized and unfunctionalized alkyl, alkoxy, cycloalkyl, alkenyl, cycloalkenyl, and aryl groups, wherein any of the before mentioned groups may be with or without heteroatoms, and mixtures thereof;
each Q is independently selected from the group consisting of functionalized or unfunctionalized alkylene, cycloalkylene, and combinations thereof, wherein any of the functionalized or unfunctionalized alkylene groups may be with or without heteroatoms, and mixtures thereof;
each E is independently selected from the group consisting of —OM, —OR₇, —NHR₇, —NR₇R₁₁, and mixtures thereof; and
each M is independently selected from the group consisting of hydrogen, alkali metal ions, alkaline earth metal ions, ammonium ions, and mixtures thereof.

The multifunctional polymer may have a weight-average molecular weight ranging from about 1,000 Da to about 300,000 Da, more particularly from about 1,000 Da to about 50,000 Da; yet more particularly from about 1,000 Da to about 30,000 Da; yet more particularly from about 1,000 Da to about 15,000 Da; and most particularly from about 1,000 Da to about 10,000 Da. The molecular weight may be determined, in part, based on the addition level of the multifunctional polymer, multifunctional polymer type, rheology considerations, and desired level of antimicrobial activity.

Selection of the generic substituent R′ and R″, R₅, R₆, R₇, R₈, R₉, R₁₀, R₁₁, Q, and M provides polymers that exhibit antimicrobial activity as well functionality for formulated compositions.

The multifunctional polymers provided herein may be synthesized from at least three methods. By a first method, one or more pre-formed polymers having at least one anhydride moiety are functionalized by a grafting reaction with at least two reactants. The first reactant has a pseudo-cationic moiety and a group that is reactive to the anhydride moiety. The second reactant helps impart and/or modulate hydrophobic character to the multifunctional polymer, and it also has a group that is reactive to the anhydride moiety. In addition to the first method, a second method also may be employed, wherein at least one anhydride-containing monomer is reacted with the abovementioned first and second reactants, and then polymerized. The third method of synthesis is a combination of the first and second methods, i.e., at least one anhydride-containing monomer is functionalized by the first and/or second reactant, a polymerization is conducted with at least that anhydride-containing monomer, and then after polymerization, additional functionalization reactions are performed.

A brief description first is given of the anhydride-containing monomer, followed by the first and second reactants to help illustrate non-limiting aspects of this invention. Then, a generalized structure of the multifunctional polymers conforming to some embodiments is provided.

The anhydride-containing monomer may be any polymerizable anhydride. Particularly, the anhydride-containing monomer may be maleic anhydride, methyl maleic anhydride, dimethyl maleic anhydride, itaconic anhydride, citraconic anhydride, and/or tetrahydrophthalic anhydride, and their functionalized analogues.

The first reactant having a pseudo-cationic moiety and a group reactive to the anhydride moiety may be represented by the structure: X-Q-R₅, wherein

X is —OH or —NR₆,

R₅ may be selected from the group consisting of —NR₉R₁₀, functionalized and unfunctionalized nitrogen or phosphorus containing C₅-C₇ cyclic groups, and mixtures thereof;
R₆ may be selected from the group consisting of hydrogen, functionalized and unfunctionalized alkyl, alkoxy, cycloalkyl, alkenyl, cycloalkenyl, aryl groups, wherein any of the before mentioned groups may be with or without heteroatoms, and mixtures thereof; and
R₉ and R₁₀ may be independently selected from the group consisting of hydrogen, functionalized and unfunctionalized alkyl, alkoxy, cycloalkyl, alkenyl, cycloalkenyl, aryl groups, wherein any of the before mentioned groups may be with or without heteroatoms, and mixtures thereof.

With regard to the first reactant, the term “pseudo-cationic moiety” refers to the —R₅ moiety, wherein the nitrogen or phosphorus atoms are capable of being protonated to form a transient positively-charged species. The term “reactive to the anhydride moiety” refers to the —X group.

More particularly, X may be —NR₆, and R₆ may be hydrogen. In yet another aspect, Q may be a functionalized or unfunctionalized C₁-C₄ alkylene and/or cycloalkylene group. Possible choices for the first reactant include, but are not limited to, the following compounds:

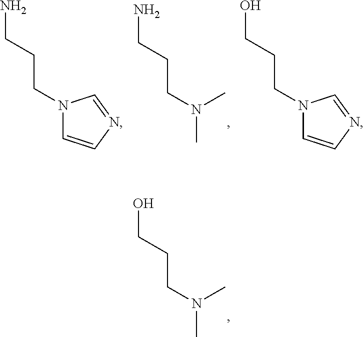

and combinations of these reactants may be employed. Of course, one skilled in the art may devise other choices for this first reactant in accordance with the generic structure outlined above.

In addition to a first reactant, at least one second reactant also is employed that may impart hydrophobic character to the grafted polymer. This second reactant has a group reactive to the anhydride moiety. In one aspect, this second reactant may be represented by the structures:

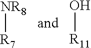

wherein
R₇ and R₁₁ may be independently selected from the group consisting of functionalized and unfunctionalized alkyl, alkoxy, cycloalkyl, alkenyl, cycloalkenyl, and aryl groups, wherein any of the before mentioned groups may be with or without heteroatoms, and mixtures thereof; and
R₈ may be independently selected from the group consisting of hydrogen, functionalized and unfunctionalized alkyl, alkoxy, cycloalkyl, alkenyl, cycloalkenyl, aryl groups, wherein any of the before mentioned groups may be with or without heteroatoms, and mixtures thereof.

More particularly, R₇ may be a functionalized or unfunctionalized C₁-C₂₂ alkyl group, and R₈ may be hydrogen. Independently, R₁₁ may be a functionalized or unfunctionalized C₁-C₂₂ alkyl group. The Examples illustrate some of the possible choices for the second reactant, including the following compounds:

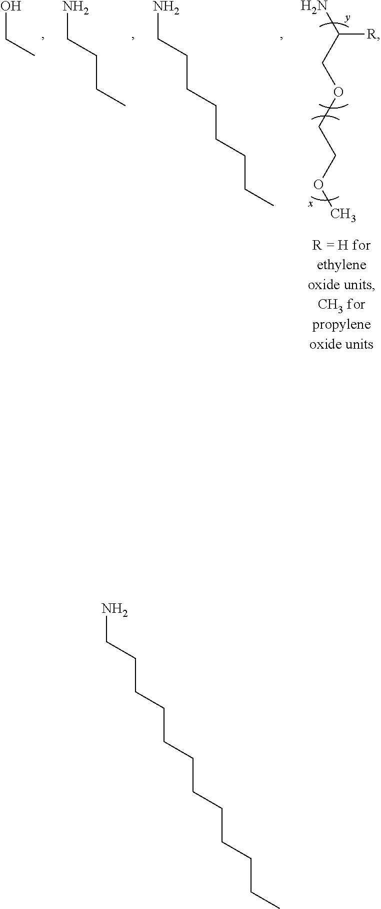

and combinations of these reactants may be employed. Of course, one skilled in the art may devise other choices for this first reactant in accordance with the generic structure outlined above.

Mention is made that the second reactant may be a solvent in which the grafting reaction occurs. For example, a lower molecular weight alcohol may be used. The Examples illustrate the use of ethanol in this capacity. Ethyl ester grafts may help provide sufficient hydrophobicity to assist in solubilizing the polymer in alcohols and/or other solvents.

Returning now to the description of the first method, the first and second reactants may react with a pre-formed polymer having at least one anhydride moiety. Examples of this pre-formed, anhydride-containing polymer include polymers of maleic anhydride, methyl maleic anhydride, dimethyl maleic anhydride, itaconic anhydride, citraconic anhydride, and tetrahydrophthalic anhydride, as well as their functionalized analogues. The pre-formed polymer may be a homopolymer, such as poly(maleic anhydride), poly(dimethyl maleic anhydride), poly(methyl maleic anhydride), poly(citraconic anhydride), and poly(tetrahydrophthalic anhydride), as well as their functionalized analogues. Disclosure of these homopolymers is made in the following documents, each of which is incorporated in its entirety by reference: U.S. Pat. Nos. 3,359,246; 3,385,834; and GB 1,120,789.

Alternatively, the anhydride-containing polymer may be a pre-formed non-homopolymer polymerized from an anhydride-containing monomer with one or more other monomers. The non-anhydride monomer(s) may be selected among alpha-olefins, vinyl ethers, styrenes, (meth)acrylates, (meth)acrylamides, 4-vinyl-1,2,3-triazoles, 5-vinyl-1,2,3-triazoles, vinyls, allyls, maleates, maleimides, α-β-olefinically unsaturated carboxylic nitriles, vinyl esters, vinyl acetates, vinyl amides, vinyl alcohols, vinyl carbonates, vinyl carbamates, vinyl thiocarbamates, vinyl ureas, vinyl halides, vinyl imidazoles, vinyl lactams, vinyl pyridines, vinyl silanes, vinyl siloxanes, vinyl sulfones, allyl ethers, and combinations thereof. For example, a non-anhydride monomer may be iso-butylene, 1-decene, styrene, methyl vinyl ether, ethyl vinyl ether, iso-propyl vinyl ether, n-butyl vinyl ether, iso-butyl vinyl ether, sec-butyl vinyl ether, octyl vinyl ether, decyl vinyl ether, dodecyl vinyl ether, hexadecyl vinyl ether, octadecyl vinyl ether, and combinations thereof.

In one embodiment, the pre-formed non-homopolymer may be poly(styrene-co-maleic anhydride), which is a general class of alternating copolymers of styrene and maleic anhydride, or the non-equimolar copolymers containing less than about 50 mole percent of the anhydride monomer. This copolymer may be represented by the structure:

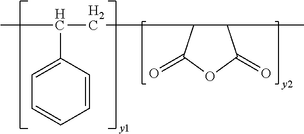

wherein the subscripts y₁ and y₂ represent the molar ratios of the two constituent blocks. This copolymer is available for purchase from Sigma Aldrich in a variety of molecular weights ranging from a number-average molecular weight (Mₙ) of 1,600 Da to about 350,000 Da.

As a further example, multifunctional polymers may be prepared by the grafting of dimethylaminopropylamine and ethanol onto alternating poly(styrene-co-maleic anhydride), e.g., having a (Mₙ) of 1,600 Da to about 350,000 Da:

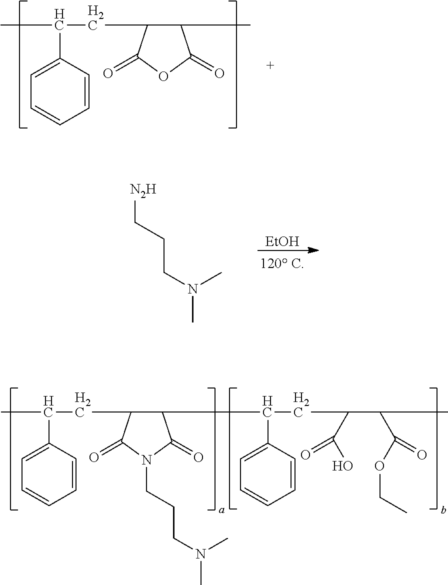

where the subscripts a and b are the molar ratios of the grafted reaction products.

Other multifunctional polymers may be prepared, for example, by replacing the pre-formed poly(iso-butylene-co-maleic anhydride) polymer of Examples 1-43 with poly(styrene-co-maleic anhydride). Furthermore, other pseudo-cationic moieties and/or hydrophobic moieties also may be used.

The styrene constituent in poly(styrene-co-maleic anhydride) may be replaced in whole or in part by other vinyl aromatic monomers such as α-methyl styrene, ethyl styrene, iso-propyl styrene, tert-butyl styrene, chlorostyrenes, dichlorostyrenes, bromostyrenes, dibromostyrenes, vinylnaphthalene and the like. Similarly, the maleic anhydride can be replaced in whole or in part by another alpha, beta-unsaturated cyclic dicarboxylic acid anhydride such as citraconic, chloromaleic, bromomaleic, dichloromaleic, dibromomaleic, phenylmaleic, and the like. The preferred α,β-unsaturated cyclic anhydride is maleic anhydride. As is the case with all other non-homopolymers, this polymer also may contain a termonomer, such as 1-3 carbons alkyl acrylate or methacrylate, acrylonitrile, methacrylonitrile, acrylamide, methacrylamide, acrylic acid or methacrylic acid, or any of the optional polymerizable groups presented later.

Suitable poly(styrene-co-maleic anhydride) copolymers may be prepared by any of the several methods available for the preparation of styrene-maleic anhydride copolymers or they may be purchased commercially. Non-equimolar copolymers may be prepared by solution polymerization directly from the respective monomers by the incremental addition of the reactive monomer as taught by U.S. Pat. No. 2,971,939, by a continuous recycle polymerization process such as described in U.S. Pat. Nos. 2,769,804 and 2,989,517, by the suspension polymerization process described in U.S. Pat. No. 3,509,110, or by numerous known variations.

In another embodiment, the pre-formed non-homopolymer may be a poly(alkyl vinyl ether-co-maleic anhydride), including those wherein the alkyl ether group may comprise from 1 to 20 carbon atoms. When the alkyl ether group contains 1 carbon atom, it may be known as a poly(methyl vinyl ether-co-maleic anhydride), including those offered into commercial sale under the trade name of Gantrez™ by Ashland Specialty Ingredients.

In yet another aspect, the pre-formed non-homopolymer may be a poly(iso-butylene-co-maleic anhydride). These polymers are available in a wide range of molecular weights, including those having a weight-average molecular weight of about 6,000 Da up to 240,000 Da and more. Poly(iso-butylene-co-maleic anhydride) polymers are available from Sigma.

A known, functionalized poly(iso-butylene-co-maleic anhydride) polymer is polyimide-1, also known by its trade name Aquaflex™ XL-30. It is the dimethylaminopropyl imide, poly(ethylene oxide/propylene oxide)imide, ethyl ester of poly(iso-butylene-co-maleic anhydride). It has a molecular weight of about 70,000 Da and is known as a film former giving clear, thick gels, and finds use in hair care applications. This polymer is disclosed in the product brochure, “Aquaflex® XL-30, A Volumizing Styling Resin with Long Lasting Hold,” International Specialty Products, May 2003.

Where a pre-formed polymer is functionalized with at least one first reactant and at least one second reactant, the pre-formed polymer may have a weight-average molecular weight ranging from about 1,000 Da to about 300,000 Da, more particularly from about 1,000 Da to about 50,000 Da; yet more particularly from about 1,000 Da to about 30,000 Da; yet more particularly from about 1,000 Da to about 15,000 Da; and most particularly from about 1,000 Da to about 10,000 Da. The amphiphilic characteristics of the multifunctional polymer may be fine-tuned by adjusting the type of hydrophobe, hydrophobicity/hydrophilicity balance, and molecular weight of the polymer.

As mentioned earlier, a second synthesis method is available that may be employed in the preparation of the multifunctional polymers. Compounds of the invention may be prepared by first reacting an anhydride-containing monomer with at least a first reactant and at least a second reactant, and then polymerizing the anhydride monomer. The anhydride-containing monomers, first and second reactant retain the non-limiting description provided for the first method.

In yet another embodiment, multifunctional polymers may be prepared by a third method, which is a combination of the first two methods. For example, an anhydride-containing monomer may be partly functionalized with a first and/or second reactants, and then the monomer polymerized by itself (to produce an homopolymer) or with other monomers (to yield a non-homopolymer). Then, that polymer product may be functionalized with first and/or second reactants to generate the multifunctional polymer.

Regardless of the synthesis approach (method 1, 2, or 3), the imide forms can be created from the maleamic acid form through the application of heat, the use of a reaction catalyst, or the use of a reaction initiator, or combinations thereof.

In another embodiment, the multifunctional polymers may be represented by the structure:

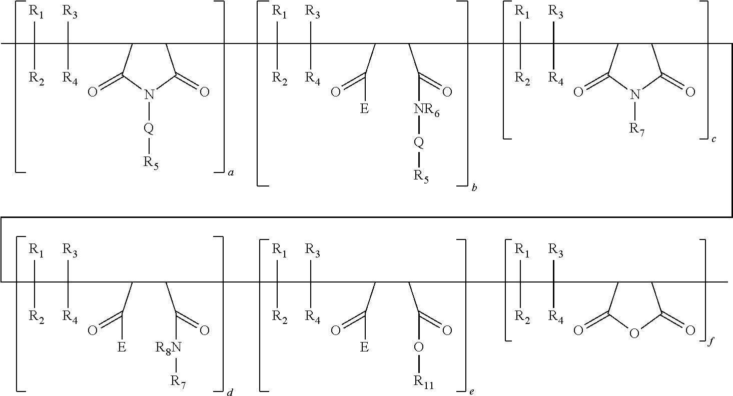

wherein each R₁, R₂, R₃, R₄, R₆, R₈, R₉, and R₁₀ is independently selected from the group consisting of hydrogen, functionalized and unfunctionalized alkyl, alkoxy, cycloalkyl, alkenyl, cycloalkenyl, aryl groups, wherein any of the before mentioned groups may be with or without heteroatoms, and mixtures thereof;
each R₅ is independently selected from the group consisting of —NR₉R₁₀, functionalized and unfunctionalized nitrogen or phosphorus containing C₅-C₇ cyclic groups, and mixtures thereof;
each R₇ and R₁₁ is independently selected from the group consisting of functionalized and unfunctionalized alkyl, alkoxy, cycloalkyl, alkenyl, cycloalkenyl, and aryl groups, wherein any of the before mentioned groups may be with or without heteroatoms, and mixtures thereof;
each Q is independently selected from the group consisting of functionalized or unfunctionalized alkylene, cycloalkylene, and combinations thereof, wherein any of the functionalized or unfunctionalized alkylene groups may be with or without heteroatoms, and mixtures thereof;
each E is independently selected from the group consisting of —OM, —OR₇, —NHR₇, —NR₇R₁₁, and mixtures thereof;
each M is independently selected from the group consisting of hydrogen, alkali metal ions, alkaline earth metal ions, ammonium ions, and mixtures thereof; and
a, b, c, d, e, and f are mole percents whose sum in each polymer equals 100%, with the proviso that at least one of a and b is not zero; and at least one of c, d, and e is not zero, wherein the polymer is alternating, block, or random.

While the generic polymer structure illustrated immediately above may seem suggesting copolymers from only two different monomers, the invention embraces multifunctional polymers polymerized from more than two monomer types (i.e., different choices of R₁-R₄ each).

In one aspect, wherein each R₁, R₂, R₃, and R₄ independently may be selected from the group consisting of hydrogen, alkyl, and aryl groups, wherein any of the before mentioned groups may be with or without heteroatoms, and mixtures thereof; each R₁, R₂, R₃, and R₄ independently may be selected from the group consisting of hydrogen, methyl, ethyl, phenyl, methoxy, and ethoxy groups; each R₅ independently may be selected from the group consisting of —NR₉R₁₀, functionalized and unfunctionalized nitrogen containing C₅-C₇ cyclic groups, and di-(C₁-C₈ alkyl)amino group or imidazolyl groups wherein any of the before mentioned groups may be with or without heteroatoms, and mixtures thereof; each Q may be selected independently from functionalized or unfunctionalized C₁-C₄ alkylene groups; each R₇ is independently a functionalized or unfunctionalized C₁-C₉ alkyl group; each R₁₁ may be independently a functionalized or unfunctionalized C₁-C₇ alkyl group.

The multifunctional polymers described herein may have a weight-average molecular weight of less than 300,000 Da, more particularly the weight-average molecular weight is less than 80,000 Da, and yet more particularly the weight-average molecular weight is less than 50,000 Da.

In one embodiment, the multifunctional polymers express antimicrobial activity against a microbe, including bacteria, fungi, and/or protozoa such S. aureus, E. coli, P. aeruginosa, A. niger, C. albicans, and mixtures thereof. In a separate embodiment, the multifunctional polymers express antimicrobial activity at a microorganism concentration of 10⁵-10⁶ cfu/mL and a polymer concentration of 1% (w/w) or greater.

Non-limiting examples of the multifunctional polymers embraced by the invention include:

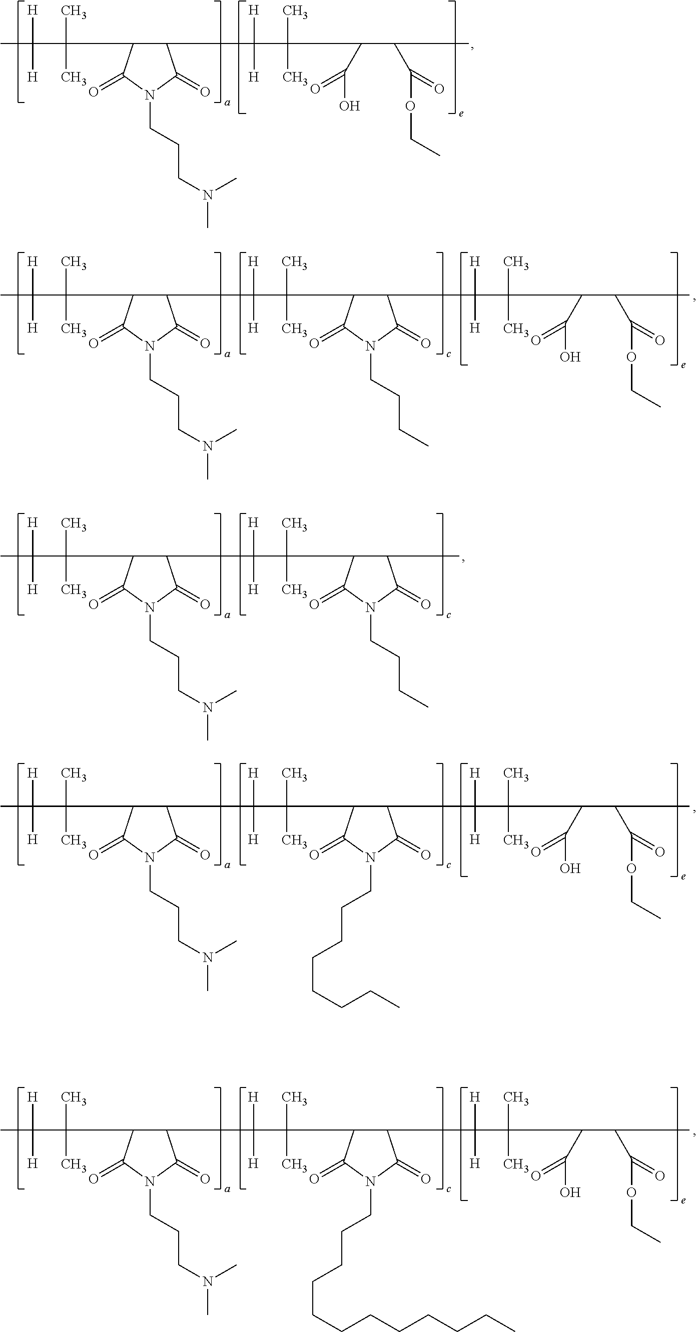

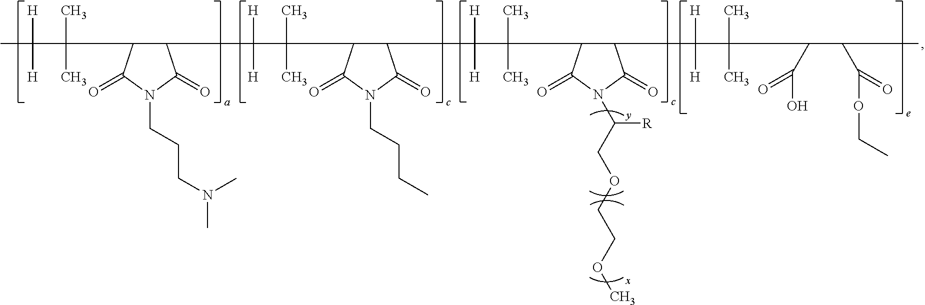

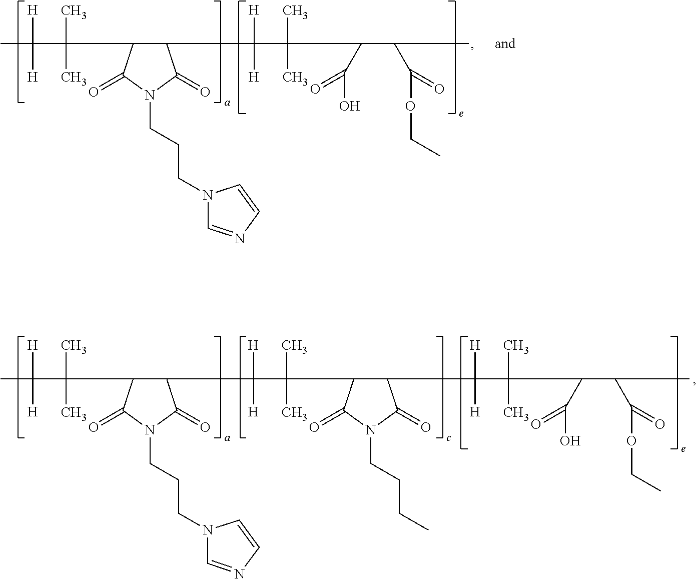

wherein the subscripts a, b, c, d, and e are molar ratios whose sum in each polymer equal 100%.

As set out above, the maleic anhydride based polymer may be partially or fully ring-opened to provide amic acids, carboxylic acids, carboxylic acid salts, imides, esters, and mixtures thereof. The partially or fully ring-opened polymers, and mixtures thereof, can be converted to a variety of useful polymers having a wide variety of physical and mechanical properties to suit a particular application. The polymers may be random, block, or alternating polymers. The properties of the multifunctional polymers can be further designed by appropriate selection of the types of polymers employed, the ratios of the polymers and the degree and type of ring opening, and the hydrophilic/hydrophobic amino functionalities to provide the desired physical properties of the multifunctional polymers including the hydrophilic, hydrophobic, and mechanical properties.

Also embraced by the invention is a method of providing antimicrobial activity in or on a composition, wherein the method comprising the step: contacting a composition with at least one multifunctional polymer comprising:

(A) at least a first repeating unit selected from the group consisting of:

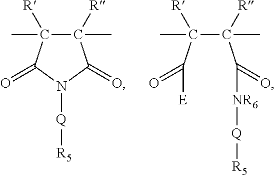

and combinations thereof, and
(B) at least a second repeating unit is selected from the group consisting of:

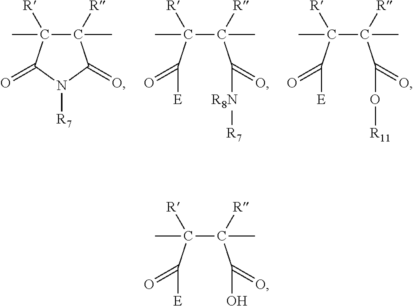

and combinations thereof,
wherein
each C— indicates a bond from said unit to another unit along the polymer backbone;
each R′ and R″ is independently selected from the group consisting of: hydrogen, alkyl, cycloalkyl, aryl, and combinations thereof;
each R₅ is independently selected from the group consisting of —NR₉R₁₀, functionalized and unfunctionalized nitrogen or phosphorus containing C₅-C₇ cyclic groups, and mixtures thereof;
each R₆, R₈, R₉, and R₁₀ is independently selected from the group consisting of hydrogen, functionalized and unfunctionalized alkyl, alkoxy, cycloalkyl, alkenyl, cycloalkenyl, aryl groups, wherein any of the before mentioned groups may be with or without heteroatoms, and mixtures thereof;
each R₇ and R₁₁ is independently selected from the group consisting of functionalized and unfunctionalized alkyl, alkoxy, cycloalkyl, alkenyl, cycloalkenyl, and aryl groups, wherein any of the before mentioned groups may be with or without heteroatoms, and mixtures thereof;
each Q is independently selected from the group consisting of functionalized or unfunctionalized alkylene, cycloalkylene, and combinations thereof, wherein any of the functionalized or unfunctionalized alkylene groups may be with or without heteroatoms, and mixtures thereof;
each E is independently selected from the group consisting of —OM, —OR₇, —NHR₇, —NR₇R₁₁, and mixtures thereof;
each M is independently selected from the group consisting of hydrogen, alkali metal ions, alkaline earth metal ions, ammonium ions, and mixtures thereof.

The multifunctional polymer for this method may have a weight-average molecular weight ranging from about 1,000 Da to about 300,000 Da, more particularly from about 1,000 Da to about 50,000 Da; yet more particularly from about 1,000 Da to about 30,000 Da; yet more particularly from about 1,000 Da to about 15,000 Da; and most particularly from about 1,000 Da to about 10,000 Da. The molecular weight may be determined, in part, based on the addition level of the multifunctional polymer, multifunctional polymer type, rheology considerations, and desired level of antimicrobial activity.

The aforementioned method comprises the step, “contacting a composition with a multifunctional polymer” meaning that the composition may be molecular blend, a nano/micro/macroscopic dispersion, and/or nano/micro/macroscopic emulsion with one or more multifunctional polymer(s). Additionally, the composition may contact one or more multifunctional polymer(s) at an interface, e.g., as a film, in one or more layers, and/or along a phase boundary.

In one embodiment, the method provides antimicrobial activity against a microbe selected from the group consisting of S. aureus, E. coli, P. aeruginosa, A. niger, C. albicans, and mixtures thereof. In a separate embodiment, the multifunctional polymers express antimicrobial activity at a microorganism concentration of 10⁵-10⁶ cfu/mL and a polymer concentration of 1% (w/w) or greater.

The polymer that may be used in the method may be a homopolymer, or it may be a non-homopolymers as described earlier. A suitable non-homopolymer may be polymerized to have one or more of the functionalized anhydride-containing units described above with one or more other repeating units. These other repeating units are the polymerized residue of one or more monomers selected from the group consisting of: alpha-olefins, vinyl ethers, styrenes, (meth)acrylates, (meth)acrylamides, styrenes, 4-vinyl-1,2,3-triazoles, 5-vinyl-1,2,3-triazoles, vinyls, allyls, maleates, maleimides, α-β-olefinically unsaturated carboxylic nitriles, vinyl esters, vinyl acetates, vinyl amides, vinyl alcohols, vinyl carbonates, vinyl carbamates, vinyl thiocarbamates, vinyl ureas, vinyl halides, vinyl imidazoles, vinyl lactams, vinyl pyridines, vinyl silanes, vinyl siloxanes, vinyl sulfones, allyl ethers, and combinations thereof. In one regard, the alpha-olefin may be iso-butylene, 1-decene, methyl vinyl ether, ethyl vinyl ether, iso-propyl vinyl ether, n-butyl vinyl ether, iso-butyl vinyl ether, sec-butyl vinyl ether, octyl vinyl ether, decyl vinyl ether, dodecyl vinyl ether, hexadecyl vinyl ether, octadecyl vinyl ether, and combinations thereof.

When non-homopolymers are employed in the method, they may be represented by the structure:

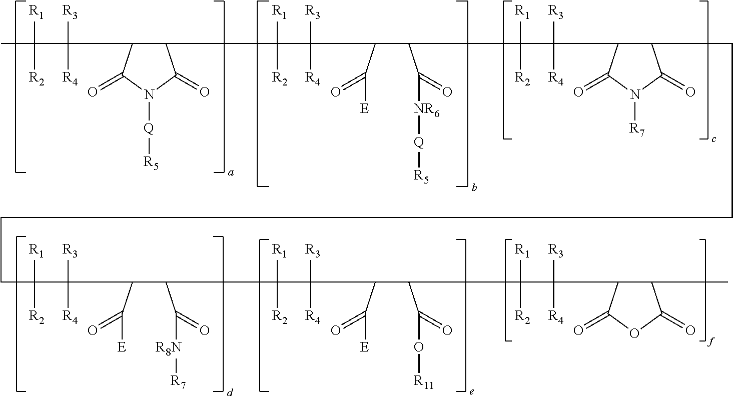

wherein each R₁, R₂, R₃, R₄, R₆, R₈, R₉, and R₁₀ is independently selected from the group consisting of hydrogen, functionalized and unfunctionalized alkyl, alkoxy, cycloalkyl, alkenyl, cycloalkenyl, aryl groups, wherein any of the before mentioned groups may be with or without heteroatoms, and mixtures thereof;
each R₅ is independently selected from the group consisting of —NR₉R₁₀, functionalized and unfunctionalized nitrogen or phosphorus containing C₅-C₇ cyclic groups, and mixtures thereof;
each R₇ and R₁₁ is independently selected from the group consisting of functionalized and unfunctionalized alkyl, alkoxy, cycloalkyl, alkenyl, cycloalkenyl, and aryl groups, wherein any of the before mentioned groups may be with or without heteroatoms, and mixtures thereof;
each Q is independently selected from the group consisting of functionalized or unfunctionalized alkylene, cycloalkylene, and combinations thereof, wherein any of the functionalized or unfunctionalized alkylene groups may be with or without heteroatoms, and mixtures thereof;
each E is independently selected from the group consisting of —OM, —OR₇, —NHR₇, —NR₇R₁₁, and mixtures thereof;
each M is independently selected from the group consisting of hydrogen, alkali metal ions, alkaline earth metal ions, ammonium ions, and mixtures thereof; and
a, b, c, d, e, and f are mole percents whose sum in each polymer equals 100%, with the proviso that at least one of a and b is not zero; and at least one of c, d, and e is not zero, wherein said polymer is alternating, block, or random.

Examples of such polymers include:

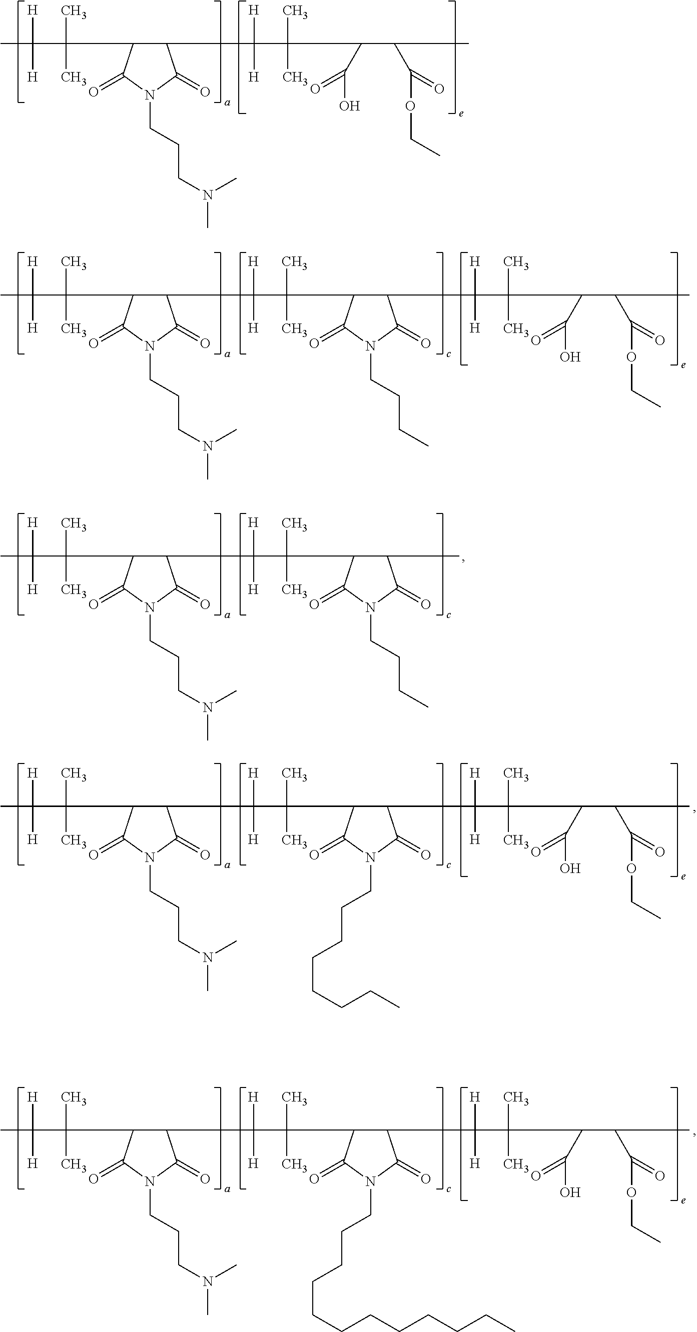

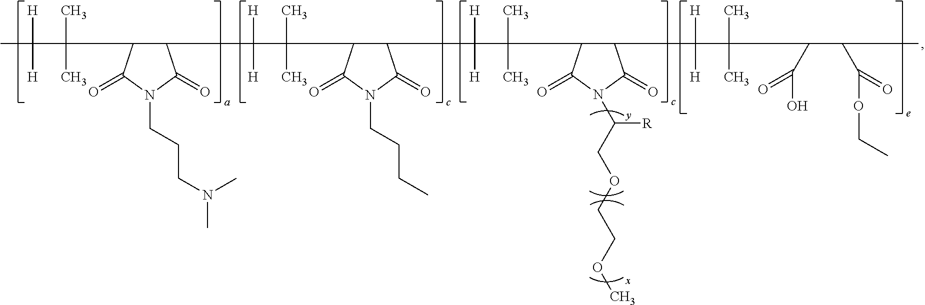

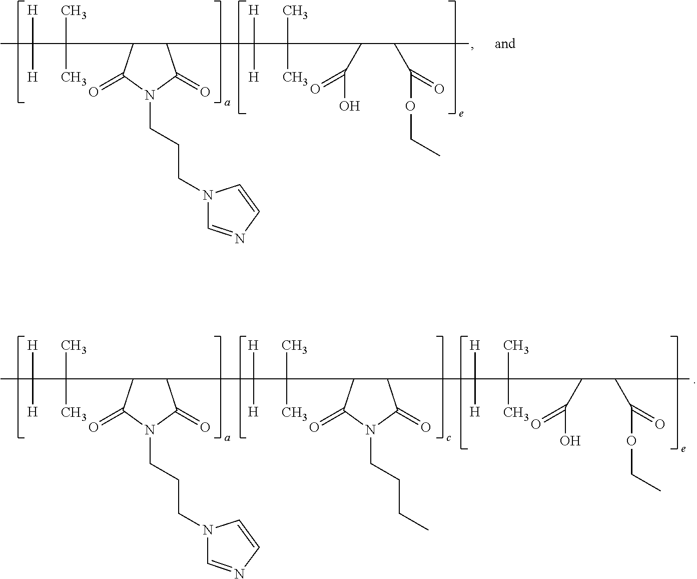

wherein the subscripts a, b, c, d, and e are molar ratios whose sum in each polymer equal 100%.

Polymers of the invention may be used in any compositions that might benefit from their functionalized properties. A few examples of the compositions include: such as in adhesives, agricultural, biocides, coatings, electronics, household-industrial-institutional (HI&I), inks, membranes, metal fluids, oilfield, paper, personal care, paints, plastics, printing, plasters, and wood-care compositions.

Depending on the end application, one or more fillers may be included in the compositions and may be added for improved rheological properties and/or stress reduction. Examples of suitable nonconductive fillers include alumina, aluminum hydroxide, silica, fused silica, fumed silica, vermiculite, mica, wollastonite, calcium carbonate, titania, sand, glass, barium sulfate, zirconium, carbon black, organic fillers, and halogenated ethylene polymers, such as, tetrafluoroethylene, trifluoroethylene, vinylidene fluoride, vinyl fluoride, vinylidene chloride, and vinyl chloride. Examples of suitable conductive fillers include carbon black, graphite, gold, silver, copper, platinum, palladium, nickel, aluminum, silicon carbide, boron nitride, diamond, and alumina. Combinations of these fillers may be used.

The filler particles may be of any appropriate size, particularly from the nano to micro range. The choice of such size for any particular end use is within the expertise of one skilled in the art. The filler may be present in an amount from about 10% to about 90% by weight of the total composition. More than one filler type may be used in a composition and the fillers may or may not be surface treated. Appropriate filler sizes can be determined by the practitioner, and, in particular, may be within the range from about 20 nm to about 100 μm.

Other materials, such as adhesion promoters (e.g. epoxides, silanes), dyes, pigments, and rheology modifiers may be added as desired for the modification of the final properties. Such materials and the amounts needed are within the expertise of those skilled in the art.

Compositions belonging to the personal care/cosmetic and pharmaceutical arts find utility in altering, delivering an active, enhancing, improving, modifying the appearance, condition, color, health, style of the skin (including face, scalp, and lips), hair, nails, and oral cavity. Many examples and product forms of these compositions are known. These compositions can impart benefits that include, but are not limited to, hair style flexibility, hair style durability, humidity resistance for hair, color and/or color protection, moisturization, wrinkle reduction, protection from ultraviolet radiation, water proofness, water resistance, wear resistance, thermal protection, adhesion, active ingredient delivery, anti-cavity, and/or anti-gingivitis protection. As such, these compositions are sometimes categorized in the following areas: skin care, hair care (both styling and non-styling), sun care, cosmetics (including color cosmetics), antiperspirants, deodorants, oral hygiene, and men's and women's personal hygiene/grooming. In some cases these benefits and care areas overlap with another.

Skin care compositions include those materials used on the body, face, hands, lips, and/or scalp, and are beneficial for many reasons, such as firming, anti-cellulite, moisturizing, nourishing, cleaning, reducing or eliminating the appearance of wrinkles or lentigo, toning, and/or purifying. They also can be used to sanitize.

Consumers can identify many of the compositions that serve the sun care area, for example after-fun, children's, beach, self-tan, sports (i.e., being sweat proof, waterproof, resistant to running, or having added UV absorbers and/or antioxidants), sensitive skin products (i.e., having low irritation to the eyes and/or skin, and/or being free of fragrances and/or dyes), daily wear, leave-on hair creams, lotions, styling products, and hair sprays. Typically, sun care products also comprise one or more UV actives, which are those organic and inorganic materials that scatter, absorb, and/or reflect radiation having a wavelength from about 100 nm to about 400 nm. In one aspect, the sun care product protects against UV-A and/or UV-B radiation. UV-A radiation, from about 320 nm to about 400 nm, has the longest wavelength within the UV spectrum, and consequently is the least energetic. While UV-A rays can induce skin tanning, they are liable to induce adverse changes as well, especially in the case of sensitive skin or of skin, which is continually exposed to solar radiation. In particular UV-A rays cause a loss of skin elasticity and the appearance of wrinkles, leading to premature skin aging. UV-B rays have shorter wavelengths, from about 290 nm to about 320 nm, and their higher energy can cause erythema and skin burns, which may be harmful. Alternatively, sun care products may omit UV actives, and may be regarded as a tanning oil or a tan promoter. Some sun care compositions may promote soothe skin after sun exposure, and/or be formulated for application to the lips, hair, or the area around the eyes. Self-tan compositions, which are products that color skin without requiring full sun exposure, also fit under the sun care umbrella. The many different sun care product formats include may assume a consistency ranging from liquid to semi-liquid forms (e.g., milks, creams), to thicker forms like gels, creams, pastes, and even solid- and wax-like forms. Sun care products also may take the form of an aerosol, spray, mist, roll-on, or wipe.

Hair care compositions include shampoos, leave-on and rinse-out conditioners used for conditioning, moisturizing, repairing, hair colors, hair relaxers, and deep conditioners and treatments such as hot oils and waxes, 2-in-1 shampoo/conditioner combination products, 3-in-1 shampoo/conditioner/styling agent. The many types of hair care products can be delivered in an array of formats, including aerosol sprays, pump sprays, gel sprays, mousses, gels, waxes, creams, pomades, spritzes, putties, lacquers, de-frizzing serums, perms, relaxants and colorants.

Color cosmetic compositions include facial make-up, eye makeup, mascaras, lip and nail products. Facial make-up compositions include foundation (liquid, solid, and semi-solid)—skin tinted creams, liquid, sticks, mousses used as a base under make-up, rouge, face powder, blusher, highlighters, face bronzers, concealers, and 2-way cake products.

Personal care/cosmetics also include eye make-up, mascaras, eyeliners, eye shadows, eyebrow pencils and eye pencils. Lip products include lipsticks, lip pencils, lip gloss, transparent bases and tinted lip moisturizers as well as multi-function color sticks that can also be used for cheeks and eyes. Nail products include nail varnishes/enamels, nail varnish removers, treatments, home-manicure products such as cuticle softeners and nail strengtheners.

In addition to the skin, hair, and sun care compositions summarized above, the polymers related herein also find application in oral care compositions. Non-limiting examples or oral care compositions include toothpastes (including toothpaste gels), denture adhesives, whiteners, anesthetics, and dental floss and related products. These compositions may take any product format, such as pastes, gels, creams, solutions, dispersions, rinses, flosses, aerosols, powders, and lozenges.

Grooming products for men and women include shaving products and toiletries, which may find use in preparing the skin and/or hair for dry or wet shaving. In addition, these compositions may help to moisturize, cool, and/or soothe skin. A variety of product forms are known, a few of which are foams, gels, creams, sticks, oils, solutions, tonics, balms, aerosols, mists, sprays, and wipes.

The polymer can also be used in other personal care/cosmetic applications, such as an absorbent material in appropriate applications such as diapers, incontinence products, feminine products, and other related products.

The polymers described herein also find application in bath and shower compositions, such as foams, gels, salts, oils, balls, liquids, powders and pearls. Also included are bar soaps, body washes, shower gels, cleansers, gels, oils, foams, scrubs and creams. As a natural extension of this category, these compositions also include liquid soaps and hand sanitizers used for cleaning hands.

The polymer of the invention can be used in combination with one or more additional personal care/cosmetically acceptable additives chosen from, for example, conditioning agents, protecting agents, such as, for example, hydrosoluble, liposoluble and water-insoluble UV filters, antiradical agents, antioxidants, vitamins and pro-vitamins, fixing agents, oxidizing agents, reducing agents, dyes, cleansing agents, anionic, cationic, nonionic and amphoteric surfactants, thickeners, perfumes, pearlizing agents, stabilizers, pH adjusters, filters, hydroxy acids, various cationic, anionic and nonionic polymers, cationic and nonionic polyether associative polyurethanes, preservatives, vegetable oils, mineral oils, synthetic oils, polyols such as glycols and glycerol, silicones, aliphatic alcohols, colorants, bleaching agents, highlighting agents and sequestrants.

These additives may be present in the composition according to the invention in proportions that may range from about 0% to about 20% by weight in relation to the total weight of the composition. An expert in the field according to its nature and its function may easily determine the precise amount of each additive.

Examples of these co-ingredients and many others can be found in the following references, each of which is herein incorporated in its entirety by reference: “Inventory and common nomenclature of ingredients employed in cosmetic products,” Official Journal of the European Union, 5.4.2006, pages L 97/1 through L 97/528; and International Cosmetic Ingredient Dictionary and Handbook, 13^{th} edition, ISBN: 1882621476, published by The Personal Care Products Council in January 2010.

Any known conditioning agent is useful in the personal care/cosmetic compositions of this invention. Conditioning agents function to improve the cosmetic properties of the hair, particularly softness, thickening, untangling, feel, and static electricity and may be in liquid, semi-solid, or solid form such as oils, waxes, or gums. Similarly, any known skin-altering agent is useful in the compositions of this invention. A few examples of conditioning agents include cationic polymers, cationic surfactants and cationic silicones. Conditioning agents may be chosen from synthesis oils, mineral oils, vegetable oils, fluorinated or perfluorinated oils, natural or synthetic waxes, silicones, cationic polymers, proteins and hydrolyzed proteins, ceramide type compounds, cationic surfactants, fatty amines, fatty acids and their derivatives, as well as mixtures of these different compounds.

The cationic polymers that may be used as a conditioning agent according to the invention are those known to improve the cosmetic properties of hair treated by detergent compositions. The expression “cationic polymer” as used herein, indicates any polymer containing cationic groups and/or ionizable groups in cationic groups. The cationic polymers used generally have a number-average molecular weight which falls between about 500 and 5,000,000, for example between 1000 and 3,000,000. Cationic polymers may be chosen from among those containing units including primary, secondary, tertiary, and/or quaternary amine groups that may either form part of the main polymer chain or a side chain. Useful cationic polymers include known polyamine, polyaminoamide, and quaternary polyammonium types of polymers, such as:
(1) homopolymers and copolymers derived from acrylic or methacrylic esters or amides. The copolymers can contain one or more units derived from acrylamides, methacrylamides, diacetone acrylamides, acrylic or methacrylic acids or their esters, vinyl lactams such as vinyl pyrrolidone or vinyl caprolactam, and vinyl esters. Specific examples include: copolymers of acrylamide and N,N-dimethylaminoethyl methacrylate quaternized with dimethyl sulfate or with an alkyl halide; copolymers of acrylamide and methacryloyloxyethyl trimethyl ammonium chloride; the copolymer of acrylamide and methacryloyloxyethyl trimethyl ammonium methosulfate; copolymers of vinyl pyrrolidone/dialkylaminoalkyl acrylate or methacrylate, optionally quaternized, such as the products sold under the name Gafquat™ by Ashland Specialty Ingredients; the N,N-dimethylaminoethyl methacrylate/vinyl caprolactam/vinyl pyrrolidone terpolymers, such as the product sold under the name Gaffix™ VC 713 by Ashland Specialty Ingredients; the vinyl pyrrolidone/methacrylamidopropyl dimethylamine copolymer, marketed under the name Styleze™ CC-10 by Ashland Specialty Ingredients; the vinyl pyrrolidone/quaternized dimethyl amino propyl methacrylamide copolymers such as the product sold under the name Gafquat™ HS-100 by Ashland Specialty Ingredients; and the vinyl pyrrolidone/dimethylaminopropyl methacrylamide/C₉-C₂₄ alkyldimethylaminopropyl methacrylic acid quaternized terpolymers described in U.S. Pat. No. 6,207,778 and marketed under the name Styleze™ W-20 by Ashland Specialty Ingredients.
(2) derivatives of cellulose ethers containing quaternary ammonium groups, such as hydroxyethyl cellulose quaternary ammonium that has reacted with an epoxide substituted by a trimethyl ammonium group.
(3) derivatives of cationic cellulose such as cellulose copolymers or derivatives of cellulose grafted with a hydrosoluble quaternary ammonium monomer, as described in U.S. Pat. No. 4,131,576, such as the hydroxy alkyl cellulose, and the hydroxymethyl-, hydroxyethyl- or hydroxypropyl-cellulose grafted with a salt of methacryloyl ethyl trimethyl ammonium, methacrylamidopropyl trimethyl ammonium, or dimethyl diallyl ammonium.
(4) cationic polysaccharides such as described in U.S. Pat. Nos. 3,589,578 and 4,031,307, guar gums containing cationic trialkyl ammonium groups and guar gums modified by a salt, e.g., chloride of 2,3-epoxy propyl trimethyl ammonium.
(5) polymers composed of piperazinyl units and alkylene or hydroxy alkylene divalent radicals with straight or branched chains, possibly interrupted by atoms of oxygen, sulfur, nitrogen, or by aromatic or heterocyclic cycles, as well as the products of the oxidation and/or quaternization of such polymers.
(6) water-soluble polyamino amides prepared by polycondensation of an acid compound with a polyamine. These polyamino amides may be reticulated.
(7) derivatives of polyamino amides resulting from the condensation of polyalkylene polyamines with polycarboxylic acids followed by alkylation by bi-functional agents.
(8) polymers obtained by reaction of a polyalkylene polyamine containing two primary amine groups and at least one secondary amine group with a dioxycarboxylic acid chosen from among diglycolic acid and saturated dicarboxylic aliphatic acids having 3 to 8 atoms of carbon. Such polymers are described in U.S. Pat. Nos. 3,227,615 and 2,961,347.
(9) the cyclopolymers of alkyl diallyl amine or dialkyl diallyl ammonium such as the homopolymer of dimethyl diallyl ammonium chloride and copolymers of diallyl dimethyl ammonium chloride and acrylamide.
(10) quaternary diammonium polymers such as hexadimethrine chloride. Polymers of this type are described particularly in U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945, and 4,027,020.
(11) quaternary polyammonium polymers, including, for example, Mirapol® A 15, Mirapol® AD1, Mirapol® AZ1, and Mirapol® 175 products sold by Miranol.
(12) the quaternary polymers of vinyl pyrrolidone and vinyl imidazole such as the products sold under the names Luviquat® FC 905, FC 550, and FC 370 by BASF.
(13) quaternary polyamines.
(14) reticulated polymers known in the art.

The conditioning agent can be a protein or hydrolyzed cationic or non-cationic protein. Examples of these compounds include hydrolyzed collagens having triethyl ammonium groups, hydrolyzed collagens having trimethyl ammonium and trimethyl stearyl ammonium chloride groups, hydrolyzed animal proteins having trimethyl benzyl ammonium groups (benzyltrimonium hydrolyzed animal protein), hydrolyzed proteins having groups of quaternary ammonium on the polypeptide chain, including at least one C₁-C₁₈ alkyl. Hydrolyzed proteins include Croquat™ L, in which the quaternary ammonium groups include a C₁₂ alkyl group, Croquat™ M, in which the quaternary ammonium groups include C₁₀-C₁₈ alkyl groups, Croquat™ S in which the quaternary ammonium groups include a C₁₈ alkyl group and Crotein Q in which the quaternary ammonium groups include at least one C₁-C₁₈ alkyl group. These products are sold by Croda. The conditioning agent can comprise quaternized vegetable proteins such as wheat, corn, or soy proteins such as cocodimonium hydrolyzed wheat protein, laurdimonium hydrolyzed wheat protein and steardimonium hydrolyzed wheat protein.

The conditioning agent can be a ceramide type of compound such as a ceramide, a glycoceramide, a pseudoceramide, or a neoceramide. These compounds can be natural or synthetic. Compounds of the ceramide type are, for example, described in patents pending DE4424530, DE4424533, DE4402929, DE4420736, WO95/23807, WO94/07844, EP-A-0646572, WO95/16665, FR-2 673 179, EP-A-0227994, WO 94/07844, WO 94/24097, and WO 94/10131. Ceramide type compounds useful herein include 2-N-linoleoyl amino-octadecane-1,3-diol, 2-N-oleoyl amino-octadecane-1,3-diol, 2-N-palmitoyl amino-octadecane-1,3-diol, 2-N-stearoyl amino-octadecane-1,3-diol, 2-N-behenoyl amino-octadecane-1,3-diol, 2-N-[2-hydroxy-palmitoyl]-amino-octadecane-1,3-diol, 2-N-stearoyl amino-octadecane-1,3,4-triol, N-stearoyl phytosphingosine, 2-N-palmitoyl amino-hexadecane-1,3-diol, bis-(N-hydroxy ethyl N-cetyl) malonamide, N(2-hydroxy ethyl)-N-(3-cetoxyl-2-hydroxy propyl) amide of cetylic acid, N-docosanoyl N-methyl-D-glucamine and mixtures of such compounds.

The conditioning agent can be a cationic surfactant such as a salt of a primary, secondary, or tertiary fatty amine, optionally polyoxyalkylenated, a quaternary ammonium salt, a derivative of imadazoline, or an amine oxide. Suitable examples include mono-, di-, or tri-alkyl quaternary ammonium compounds with a counter-ion such as a chloride, methosulfate, tosylate, etc. including, but not limited to, cetrimonium chloride, dicetyldimonium chloride, behentrimonium methosulfate, and the like. The presence of a quaternary ammonium compound in conjunction with the polymer described above reduces static and enhances combing of hair in the dry state. The polymer also enhances the deposition of the quaternary ammonium compound onto the hair substrate thus enhancing the conditioning effect of hair.

The conditioning agent can be any fatty amine known to be useful as a conditioning agent; e.g. dodecyl, cetyl or stearyl amines, such as stearamidopropyl dimethylamine. The conditioning agent can be a fatty acid or derivatives thereof known to be useful as conditioning agents. Suitable fatty acids include myristic acid, palmitic acid, stearic acid, behenic acid, oleic acid, linoleic acid, and isostearic acid. The derivatives of fatty acids include carboxylic esters including mono-, di-, tri- and tetra-carboxylic acids.

The conditioning agent can be a fluorinated or perfluorinated oil. Fluorinated oils include perfluoropolyethers described in EP-A-486135 and the fluorohydrocarbon compounds described in WO 93/11103. The fluoridated oils may also be fluorocarbons such as fluoramines, e.g., perfluorotributylamine, fluoridated hydrocarbons, such as perfluorodecahydronaphthalene, fluoroesters, and fluoroethers. Of course, mixtures of two or more conditioning agents can be used.

The conditioning agent can be any silicone known by those skilled in the art to be useful as a conditioning agent. The silicones suitable for use according to the invention include polyorganosiloxanes that are insoluble in the composition. The silicones may be present in the form of oils, waxes, polymers, or gums. They may be volatile or non-volatile. The silicones can be selected from polyalkyl siloxanes, polyaryl siloxanes, polyalkyl aryl siloxanes, silicone gums and polymers, and polyorgano siloxanes modified by organofunctional groups, and mixtures thereof. Suitable polyalkyl siloxanes include polydimethyl siloxanes with terminal trimethyl silyl groups or terminal dimethyl silanol groups (dimethiconol) and polyalkyl (C₁-C₂₀) siloxanes. Suitable polyalkyl aryl siloxanes include polydimethyl methyl phenyl siloxanes and polydimethyl diphenyl siloxanes, linear or branched. The silicone gums suitable for use herein include polydiorganosiloxanes including those having a number-average molecular weight between 200,000 and 1,000,000, used alone or mixed with a solvent. Examples include polymethyl siloxane, polydimethyl siloxane/methyl vinyl siloxane gums, polydimethyl siloxane/diphenyl siloxane, polydimethyl siloxane/phenyl methyl siloxane and polydimethyl siloxane/diphenyl siloxane/methyl vinyl siloxane. Suitable silicone polymers include silicones with a dimethyl/trimethyl siloxane structure and polymers of the trimethyl siloxysilicate type. The organo-modified silicones suitable for use in the invention include silicones such as those previously defined and containing one or more organofunctional groups attached by means of a hydrocarbon radical and grafted siliconated polymers. In one embodiment the silicones are amino functional silicones. The silicones may be used in the form of emulsions, nano-emulsions, or micro-emulsions.

The conditioning agent or agents can be present in an amount from about 0.001% to about 20%, particularly from about 0.01% to about 10%, and even more particularly from about 0.1% to about 3% by weight based on the total weight of the final composition. The personal care/cosmetic compositions of the invention can contain one or more protecting agents in combination with the above-described polymer to prevent or limit the degrading effects of natural physical and/or chemical assaults on the keratinous materials.

The protecting agent can be chosen from hydrosoluble, liposoluble and water-insoluble UV filters, antiradical agents, antioxidants, vitamins and pro-vitamins. The above-described cationic polymer enhances the deposition of these materials onto the hair or skin substrate enhancing protection of hair to UV damage. Organic UV filters (systems that filter out UV rays) can be chosen from among hydrosoluble or liposoluble filters, whether siliconated or nonsiliconated, and mineral oxide particles, the surface of which may be treated. Hydrosoluble organic UV filters may be chosen from para-amino benzoic acid and its salts, anthranilic acid and its salts, salicylic acid and its salts, hydroxy cinnamic acid and its salts, sulfonic derivatives of benzothiazoles, benzimidizoles, benzoxazoles and their salts, sulfonic derivatives of benzophenone and their salts, sulfonic derivatives of benzylidene camphor and their salts, derivatives of benzylidene camphor substituted by a quaternary amine and their salts, derivatives of phthalydene-camphosulfonic acids and their salts, sulfonic derivatives of benzotriazole, and mixtures thereof. Hydrophilic polymers, which have light-protective qualities against UV rays, can be used. These include polymers containing benzylidene camphor and/or benzotriazole groups.

Suitable liposoluble organic UV filters include derivatives of para-aminobenzoic acid, such as the esters or amides of para-aminobenzoic acid; derivatives of salicylic acid; derivatives of benzophenone; derivatives of dibenzoyl methane; derivatives of diphenyl acrylates; derivatives of benzofurans; UV filter polymers containing one or more silico-organic residues; esters of cinnamic acid; derivatives of camphor; derivatives of trianilino-s-triazine; the ethylic ester urocanic acid; benzotriazoles; derivatives of hydroxy phenyl triazine; bis-resorcinol-dialkyl amino triazine; and mixtures thereof. The liposoluble (or lipophilic) organic UV filter can be chosen from octyl salicylate; 4-tert-butyl-4′-methoxy dibenzoyl methane; octocrylene; 4-methoxy cinnamate; 2-ethylhexyl[2-ethylhexyl 4-methoxycinnamate]; and 2-(2H-benzotriazol-2-yl)-4-methyl-6-[2-methyl-3-[1,3,3,3-tetramethyl-1-[(trimethyl sily)oxy]disiloxanyl]propynyl]phenol. Other UV filters that may be useful are derivatives of benzophenones such as 2-hydroxy-4-methoxy benzophenone-5-sulfonic acid, 2-hydroxy-4-methoxy benzophenone, derivatives of benzalmalonates such as poly dimethyl/methyl(3(4-(2,2-bis-ethoxy carbonyl vinyl)-phenoxy)-propenyl) siloxane, derivatives of benzylidene camphor such as β-β′camphosulfonic[1-4 divinylbenzene] acid and derivatives of benzimidazole such as 2-phenyl-benzimidazol-5-sulfonic acid. Water-insoluble UV filters include various mineral oxides. The mineral oxides may be selected from among titanium oxides, zinc oxides, and cerium oxides. The mineral oxides can be used in the form of ultrafine nanoparticles. For example, the UV filters can include Escalol™ HP-610 (dimethylpabamido propyl laurdimonium tosylate and propylene glycol stearate) or Crodasorb HP (polyquaternium 59).

The antioxidants or antiradical agents can be selected from phenols such as BHA (tert-butyl-4-hydroxy anisole), BHT (2,6-di-tert-butyl-p-cresol), TBHQ (tert-butyl hydroquinone), polyphenols such as proanthocyanodic oligomers, flavonoids, hindered amines such as tetra amino piperidine, erythorbic acid, polyamines such as spermine, cysteine, glutathione, superoxide dismutase, and lactoferrin.

The vitamins can be selected from ascorbic acid (vitamin C), vitamin E, vitamin E acetate, vitamin E phosphate, B vitamins such as B3 and B5, vitamin PP, vitamin A, and derivatives thereof. The provitamins can be selected from panthenol and retinol.

The protecting agent can be present in an amount from about 0.001% to about 20% by weight, particularly from about 0.01% to about 10% by weight, and more particularly from 0.1% to about 5% by weight of the total weight of the final composition.

The composition of the invention can contain a fixing agent in combination with the above-described polymer. The fixing agent can be an anionic polymer chosen from polymers containing carboxylic units derived from unsaturated carboxylic mono- or polyacids.

The fixing agent can be an amphoteric polymer chosen from the polymer containing recurring units derived from:
a) at least one comonomer containing carboxylic acid units, and
b) at least one basic comonomer, such as esters with primary, secondary, tertiary, and quaternary amino substituents of acrylic and methacrylic acids and the product of quaternization of dimethylaminoethyl methacrylate with dimethyl or diethyl sulfate.

The fixing agent can be a nonionic polymer chosen from polyalkyloxazolines; vinyl acetate homopolymers; vinyl acetate and acrylic ester copolymers; vinyl acetate and ethylene copolymers; vinyl acetate and maleic ester copolymers; polyethylene and maleic anhydride copolymers; homopolymers of alkyl acrylates; homopolymers of alkyl methacrylates; copolymers of acrylic esters; copolymers of alkyl acrylates and alkyl methacrylates; copolymers of acrylonitrile and a nonionic monomer chosen from among butadiene and alkyl (meth)acrylates; copolymers of alkyl acrylate and urethane; and polyamides. The fixing agent can be a functionalized or unfunctionalized, silicone or non-silicone polyurethane. The fixing polymer can be a polymer of the grafted silicone type containing a polysiloxane portion and a portion consisting of a nonsilicone organic chain, with one of the two portions forming the main chain of the polymer, and with the other being grafted onto the main chain.

The fixing agent can be present in the composition in a relative weight concentration between about 0.1% to about 10%, for example, from about 0.5% to about 5%.

The personal care/cosmetic composition of the invention can contain an oxidizing agent in combination with the above-described polymer. The oxidizing agent can be chosen from the group of hydrogen peroxide, urea peroxide, alkali metal bromates, ferricyanides, persalts, and redox enzymes, optionally with their respective donor or cofactor. For example, the oxidizing agent can be hydrogen peroxide. The oxidizing agent can be a solution of oxygenated water whose titer varies from 1 to 40 volumes.

The personal care/cosmetic composition of the invention can contain at least one reducing agent in combination with the above-described polymer in amounts from about 0.01% to about 30%, particularly from about 0.05% to about 20% of the total weight of the composition. The reducing agents can be selected from thiols, like cysteine, thioglycolic acid, thiolactic acid, their salts and esters, cysteamine, and its salts or sulfites. In the case of compositions intended for bleaching, ascorbic acid, its salts and its esters, erythorbic acid, its salts and its esters, and sulfinates, like sodium hydroxymethanesulfinate can be used.

The personal care/cosmetic composition of the invention can contain a dye in combination with the above-described polymer. The dye can be selected from the group consisting of neutral acid or cationic nitrobenzene dyes, neutral acid or cationic azo dyes, quinone dyes, neutral, acid or cationic anthraquinone dyes, azine dyes, triarylmethane dyes, indoamine dyes and natural dyes. The dye or dyes can be present in a concentration from about 0.001% to about 20%, and particularly from about 0.005% to about 10% based on the total weight of the composition.

In addition, the personal care/cosmetic compositions can include at least one surfactant in combination with the above-described polymer. The surfactant can be present in an amount from about 0.1% to about 60%, particularly from about 1% to about 40%, and more particularly from about 5% to about 30% by weight based on the total weight of the composition. The surfactant may be chosen from among anionic, amphoteric, or non-ionic surfactants, or mixtures of them known to be useful in personal care/cosmetic compositions.

One or more suitable thickeners or viscosity increasing agents may be included in combination with the above-described polymer in the personal care/cosmetic compositions of the invention. Suitable thickeners and/or viscosity increasing agents include: Acetamide MEA; acrylamide/ethalkonium chloride acrylate copolymer; acrylamide/ethyltrimonium chloride acrylate/ethalkonium chloride acrylate copolymer; acrylamides copolymer; acrylamide/sodium acrylate copolymer; acrylamide/sodium acryloyldimethyltaurate copolymer; acrylates/acetoacetoxyethyl methacrylate copolymer; acrylates/beheneth-25 methacrylate copolymer; acrylates/C₁₀-C₃₀ alkyl acrylate crosspolymer; acrylates/ceteth-20 itaconate copolymer; acrylates/ceteth-20 methacrylate copolymer; acrylates/laureth-25 methacrylate copolymer; acrylates/palmeth-25 acrylate copolymer; acrylates/palmeth-25 itaconate copolymer; acrylates/steareth-50 acrylate copolymer; acrylates/steareth-20 itaconate copolymer; acrylates/steareth-20 methacrylate copolymer; acrylates/stearyl methacrylate copolymer; acrylates/vinyl isodecanoate crosspolymer; acrylic acid/acrylonitrogens copolymer; adipic acid/methyl DEA crosspolymer; agar; agarose; alcaligenes polysaccharides; algin; alginic acid; almondamide DEA; almondamidopropyl betaine; aluminum/magnesium hydroxide stearate; ammonium acrylates/acrylonitrogens copolymer; ammonium acrylates copolymer; ammonium acryloyldimethyltaurate/vinyl formamide copolymer; ammonium acryloyldimethyltaurate/VP copolymer; ammonium alginate; ammonium chloride; ammonium polyacryloyldimethyl taurate; ammonium sulfate; amylopectin; apricotamide DEA; apricotamidopropyl betaine; arachidyl alcohol; arachidyl glycol; arachis hypogaea (peanut) flour; ascorbyl methylsilanol pectinate; astragalus gummifer gum; attapulgite; avena sativa (oat) kernel flour; avocadamide DEA; avocadamidopropyl betaine; azelamide MEA; babassuamide DEA; babassuamide MEA; babassuamidopropyl betaine; behenamide DEA; behenamide MEA; behenamidopropyl betaine; behenyl betaine; bentonite; butoxy chitosan; caesalpinia spinosa gum; calcium alginate; calcium carboxymethyl cellulose; calcium carrageenan; calcium chloride; calcium potassium carbomer; calcium starch octenylsuccinate; C₂₀-C₄₀ alkyl stearate; canolamidopropyl betaine; capramide DEA; capryl/capramidopropyl betaine; carbomer; carboxybutyl chitosan; carboxymethyl cellulose acetate butyrate; carboxymethyl chitin; carboxymethyl chitosan; carboxymethyl dextran; carboxymethyl hydroxyethylcellulose; carboxymethyl hydroxypropyl guar; carnitine; cellulose acetate propionate carboxylate; cellulose gum; ceratonia siliqua gum; cetearyl alcohol; cetyl alcohol; cetyl babassuate; cetyl betaine; cetyl glycol; cetyl hydroxyethylcellulose; chimyl alcohol; cholesterol/HDI/pullulan copolymer; cholesteryl hexyl dicarbamate pullulan; citrus aurantium dulcis (orange) peel extract; cocamide DEA; cocamide MEA; cocamide MIPA; cocamidoethyl betaine; cocamidopropyl betaine; cocamidopropyl hydroxysultaine; coco-betaine; coco-hydroxysultaine; coconut alcohol; coco/oleamidopropyl betaine; coco-Sultaine; cocoyl sarcosinamide DEA; cornamide/cocamide DEA; cornamide DEA; croscarmellose; crosslinked bacillus/glucose/sodium glutamate ferment; cyamopsis tetragonoloba (guar) gum; decyl alcohol; decyl betaine; dehydroxanthan gum; dextrin; dibenzylidene sorbitol; diethanolaminooleamide DEA; diglycol/CHDM/isophthalates/SIP copolymer; dihydroabietyl behenate; dihydrogenated tallow benzylmonium hectorite; dihydroxyaluminum aminoacetate; dimethicone/PEG-10 crosspolymer; dimethicone/PEG-15 crosspolymer; dimethicone propyl PG-betaine; dimethylacrylamide/acrylic acid/polystyrene ethyl methacrylate copolymer; dimethylacrylamide/sodium acryloyldimethyltaurate crosspolymer; disteareth-100 IPDI; DMAPA acrylates/acrylic acid/acrylonitrogens copolymer; erucamidopropyl hydroxysultaine; ethylene/sodium acrylate copolymer; gelatin; gellan gum; glyceryl alginate; glycine soja (soybean) flour; guar hydroxypropyltrimonium chloride; hectorite; hyaluronic acid; hydrated silica; hydrogenated potato starch; hydrogenated tallow; hydrogenated tallowamide DEA; hydrogenated tallow betaine; hydroxybutyl methylcellulose; hydroxyethyl acrylate/sodium acryloyldimethyl taurate copolymer; hydroxyethylcellulose; hydroxyethyl chitosan; hydroxyethyl ethylcellulose; hydroxyethyl stearamide-MIPA; hydroxylauryl/hydroxymyristyl betaine; hydroxypropylcellulose; hydroxypropyl chitosan; hydroxypropyl ethylenediamine carbomer; hydroxypropyl guar; hydroxypropyl methylcellulose; hydroxypropyl methylcellulose stearoxy ether; hydroxypropyl starch; hydroxypropyl starch phosphate; hydroxypropyl xanthan gum; hydroxystearamide MEA; isobutylene/sodium maleate copolymer; isostearamide DEA; isostearamide MEA; isostearamide mIPA; isostearamidopropyl betaine; lactamide MEA; lanolinamide DEA; lauramide DEA; lauramide MEA; lauramide MIPA; lauramide/myristamide DEA; lauramidopropyl betaine; lauramidopropyl hydroxysultaine; laurimino bispropanediol; lauryl alcohol; lauryl betaine; lauryl hydroxysultaine; lauryl/myristyl glycol hydroxypropyl ether; lauryl sultaine; lecithinamide DEA; linoleamide DEA; linoleamide MEA; linoleamide MIPA; lithium magnesium silicate; lithium magnesium sodium silicate; macrocystis pyrifera (kelp); magnesium alginate; magnesium/aluminum/hydroxide/carbonate; magnesium aluminum silicate; magnesium silicate; magnesium trisilicate; methoxy PEG-22/dodecyl glycol copolymer; methylcellulose; methyl ethylcellulose; methyl hydroxyethylcellulose; microcrystalline cellulose; milkamidopropyl betaine; minkamide DEA; minkamidopropyl betaine; MIPA-myristate; montmorillonite; Moroccan lava clay; myristamide DEA; myristamide MEA; myristamide MIPA; myristamidopropyl betaine; myristamidopropyl hydroxysultaine; myristyl alcohol; myristyl betaine; natto gum; nonoxynyl hydroxyethylcellulose; oatamide MEA; oatamidopropyl betaine; octacosanyl glycol isostearate; octadecene/MA copolymer; oleamide DEA; oleamide MEA; oleamide MIPA; oleamidopropyl betaine; oleamidopropyl hydroxysultaine; oleyl betaine; olivamide DEA; olivamidopropyl betaine; oliveamide MEA; palmamide DEA; palmamide MEA; palmamide MIPA; palmamidopropyl betaine; palmitamide DEA; palmitamide MEA; palmitamidopropyl betaine; palm kernel alcohol; palm kernelamide DEA; palm kernelamide MEA; palm kernelamide MIPA; palm kernelamidopropyl betaine; peanutamide MEA; peanutamide MIPA; pectin; PEG-800; PEG-crosspolymer; PEG-150/decyl alcohol/SMDI copolymer; PEG-175 diisostearate; PEG-190 distearate; PEG-15 glyceryl tristearate; PEG-140 glyceryl tristearate; PEG-240/HDI copolymer bis-decyltetradeceth-20 ether; PEG-100/IPDI copolymer; PEG-180/laureth-50/TMMG copolymer; PEG-10/lauryl dimethicone crosspolymer; PEG-15/lauryl dimethicone crosspolymer; PEG-2M; PEG-5M; PEG-7M; PEG-9M; PEG-14M; PEG-20M; PEG-23M; PEG-25M; PEG-45M; PEG-65M; PEG-90M; PEG-115M; PEG-160M; PEG-180M; PEG-120 methyl glucose trioleate; PEG-180/octoxynol-40/TMMG copolymer; PEG-150 pentaerythrityl tetrastearate; PEG-4 rapeseed amide; PEG-150/stearyl alcohol/SMDI copolymer; phaseolus angularis seed powder; polianthes tuberosa extract; polyacrylate-3; polyacrylic acid; polycyclopentadiene; polyether-1; polyethylene/isopropyl maleate/MA copolyol; polyglyceryl-3 disiloxane dimethicone; polyglyceryl-3 polydimethylsiloxyethyl dimethicone; polymethacrylic acid; polyquaternium-52; polyvinyl alcohol; potassium alginate; potassium aluminum polyacrylate; potassium carbomer; potassium carrageenan; potassium chloride; potassium palmate; potassium polyacrylate; potassium sulfate; potato starch modified; PPG-2 cocamide; PPG-1 hydroxyethyl caprylamide; PPG-2 hydroxyethyl cocamide; PPG-2 hydroxyethyl coco/isostearamide; PPG-3 hydroxyethyl soyamide; PPG-14 laureth-60 hexyl dicarbamate; PPG-14 laureth-60 isophoryl dicarbamate; PPG-14 palmeth-60 hexyl dicarbamate; propylene glycol alginate; PVP/decene copolymer; PVP montmorillonite; pyrus cydonia seed; pyrus malus (apple) fiber; rhizobian gum; ricebranamide DEA; ricinoleamide DEA; ricinoleamide MEA; ricinoleamide MIPA; ricinoleamidopropyl betaine; ricinoleic acid/adipic acid/AEEA copolymer; rosa multiflora flower wax; sclerotium gum; sesamide DEA; sesamidopropyl betaine; sodium acrylate/acryloyldimethyl taurate copolymer; sodium acrylates/acrolein copolymer; sodium acrylates/acrylonitrogens copolymer; sodium acrylates copolymer; sodium acrylates crosspolymer; sodium acrylate/sodium acrylamidomethylpropane sulfonate copolymer; sodium acrylates/vinyl isodecanoate crosspolymer; sodium acrylate/vinyl alcohol copolymer; sodium carbomer; sodium carboxymethyl chitin; sodium carboxymethyl dextran; sodium carboxymethyl beta-glucan; sodium carboxymethyl starch; sodium carrageenan; sodium cellulose sulfate; sodium chloride; sodium cyclodextrin sulfate; sodium hydroxypropyl starch phosphate; sodium isooctylene/MA copolymer; sodium magnesium fluorosilicate; sodium oleate; sodium palmitate; sodium palm kernelate; sodium polyacrylate; sodium polyacrylate starch; sodium polyacryloyldimethyl taurate; sodium polygamma-glutamate; sodium polymethacrylate; sodium polystyrene sulfonate; sodium silicoaluminate; sodium starch octenylsuccinate; sodium stearate; sodium stearoxy PG-hydroxyethylcellulose sulfonate; sodium styrene/acrylates copolymer; sodium sulfate; sodium tallowate; sodium tauride acrylates/acrylic acid/acrylonitrogens copolymer; sodium tocopheryl phosphate; solanum tuberosum (potato) starch; soyamide DEA; soyamidopropyl betaine; starch/acrylates/acrylamide copolymer; starch hydroxypropyltrimonium chloride; stearamide AMP; stearamide DEA; stearamide DEA-distearate; stearamide DIBA-stearate; stearamide MEA; stearamide MEA-stearate; stearamide MIPA; stearamidopropyl betaine; steareth-60 cetyl ether; steareth-100/PEG-136/HDI copolymer; stearyl alcohol; stearyl betaine; sterculia urens gum; synthetic fluorphlogopite; tallamide DEA; tallow alcohol; tallowamide DEA; tallowamide MEA; tallowamidopropyl betaine; tallowamidopropyl hydroxysultaine; tallowamine oxide; tallow betaine; tallow dihydroxyethyl betaine; tamarindus indica seed gum; tapioca starch; TEA-alginate; TEA-carbomer; TEA-hydrochloride; trideceth-2 carboxamide MEA; tridecyl alcohol; triethylene glycol dibenzoate; trimethyl pentanol hydroxyethyl ether; triticum vulgare (wheat) germ powder; triticum vulgare (wheat) kernel flour; triticum vulgare (wheat) starch; tromethamine acrylates/acrylonitrogens copolymer; tromethamine magnesium aluminum silicate; undecyl alcohol; undecylenamide DEA; undecylenamide MEA; undecylenamidopropyl betaine; welan gum; wheat germamide DEA; wheat germamidopropyl betaine; xanthan gum; yeast beta-glucan; yeast polysaccharides; zea mays(corn) starch; and blends thereof.

In one such embodiment, the thickeners or viscosity increasing agents include carbomers, Aculyn™ and Stabileze™, e.g., crosslinked acrylic acid, crosslinked poly(methylvinyl ether/maleic anhydride) copolymer, acrylamides, carboxymethyl cellulose, and the like.

The personal care/cosmetic composition of the invention can contain at least one amphoteric polymer or a cationic polymer in combination with the above-described polymer. The cationic or amphoteric polymer or polymers can be present in an amount from about 0.01% to about 10%, particularly from about 0.05% to about 5%, and more particularly from about 0.1% to about 3% by weight of the total weight of the composition.

For some embodiments, it may be preferred to add one or more preservatives and/or antimicrobial agents, such as, but not limited to, benzoic acid, sorbic acid, dehydroacetic acid, piroctone olamine, DMDM hydantoin, IPBC, triclosan, bronopol, formaldehyde, isothiazolinones, nitrates/nitrites, parabens, phenoxyethanol, potassium sorbate, sodium benzoate, sulphites, and sulphur dioxide. Combinations of preservatives may be used.

In other embodiments it may be desirable to incorporate preservative boosters/solvents, select examples of which include caprylyl glycol, hexylene glycol, pentylene glycol, ethylhexylglycerin, caprylhydroxamic acid, and glyceryl caprylate.

In other embodiments it may be desirable to include one or more other ingredients, such as synthetic and natural oils and waxes. The synthetic oils include polyolefins, e.g., poly-α-olefins such as polybutenes, polyisobutenes and polydecenes. The polyolefins can be hydrogenated. The mineral oils suitable for use in the compositions of the invention include hexadecane and oil of paraffin. Suitable animal and vegetable oils include sunflower, corn, soy, avocado, jojoba, squash, raisin seed, sesame seed, walnut oils, fish oils, glycerol tricaprocaprylate, Purcellin oil or liquid jojoba. Suitable natural or synthetic oils include eucalyptus, lavender, vetiver, litsea cubeba, lemon, sandalwood, rosemary, chamomile, savory, nutmeg, cinnamon, hyssop, caraway, orange, geranium, cade, and bergamot. Suitable natural and synthetic waxes include carnauba wax, candelila wax, alfa wax, paraffin wax, ozokerite wax, vegetable waxes such as olive wax, rice wax, hydrogenated jojoba wax, absolute flower waxes such as black currant flower wax, animal waxes such as bees wax, modified bees wax (cerabellina), marine waxes and polyolefin waxes such as polyethylene wax.

The personal care/cosmetic compositions may be used to wash and treat keratinous material such as hair, skin, eyelashes, eyebrows, fingernails, lips, and hairy skin. The invention provides a method for treating keratinous material including the skin or hair, by applying to skin or keratinous materials a personal care/cosmetic composition as described above, and then eventually rinsing it with water. Accordingly, the method makes it possible to maintain the hairstyle, treatment, care, washing, or make-up removal of the skin, the hair, and any other keratinous material.

The personal care/cosmetic compositions described herein are useful in personal care/cosmetic products, including, but not limited to, gels, lotions, glazes, glues, mousses, sprays, fixatives, shampoos, conditioners, 2-in-1 shampoos, temporary hair dyes, semi-permanent hair dyes, permanent hair dyes, straighteners, permanent waves, relaxers, creams, putties, waxes, pomades, moisturizers, mascaras, lip balms and foam enhancers. The personal care/cosmetic compositions can be detergent compositions such as shampoos, bath gels, and bubble baths. In this mode, the compositions will comprise a generally aqueous washing base. The surfactant or surfactants that form the washing base may be chosen alone or in blends, from known anionic, amphoteric, or non-ionic surfactants. The quantity and quality of the washing base must be sufficient to impart a satisfactory foaming and/or detergent value to the final composition. The washing base can be from about 4% to about 50% by weight, particularly from about 6% to about 35% by weight, and even more particularly from about 8% to about 25% by weight of the total weight of the final composition. The personal care/cosmetic compositions may also take the form of after-shampoo compositions, to be rinsed off or not, for permanents, straightening, waving, dyeing, or bleaching, or the form of rinse compositions to be applied before or after dyeing, bleaching, permanents, straightening, relaxing, waving or even between the two stages of a permanent or straightening process. The personal care/cosmetic compositions may also take the form of skin-washing compositions, and particularly in the form of solutions or gels for the bath or shower, or of make-up removal products. The personal care/cosmetic compositions may also be in the form of aqueous or hydro-alcoholic solutions for skin and/or hair care.

The pH of the composition applied to the keratinous material is generally between 2 and 12. In one embodiment, the pH is from about 3 to about 8, and may be adjusted to the desired value by means of acidifying or alkalinizing agents that are well known in the state of the art. Thus, the composition of the invention can contain at least one alkalizing or acidifying agent in amounts from about 0.01% to about 30% based on the total weight of the composition.

The alkalizing agent can be chosen from ammonia, alkali hydroxides, alkali carbonates, alkanolamines, like mono-, di- and triethanolamines, as well as their derivatives, hydroxyalkylamines and ethoxylated and/or propoxylated ethylenediamines, unsubstituted and substituted propylenediamines.

The acidifying agent can be chosen from mineral or organic acids, like hydrochloric acid, orthophosphoric acid, carboxylic acids like tartaric acid, citric acid, or lactic acid, or sulfonic acids, and the like.

The personal care/cosmetic compositions of the invention may include a physiological and cosmetically acceptable medium. Such medium may consist exclusively of water, a cosmetically acceptable solvent, or a blend of water and a cosmetically acceptable solvent, such as a lower alcohol composed of C₁ to C₄, such as ethanol, isopropanol, t-butanol, n-butanol, alkylene glycols such as propylene glycol, and glycol ethers. Alternatively, the personal care/cosmetic compositions can be anhydrous.

Generally, personal care/cosmetic compositions can be prepared by simple mixing procedures well known in the art.

The invented polymers can be prepared according to the examples set out below. The examples are presented for purposes of demonstrating, but not limiting, the preparation of the compounds and compositions of this invention.

### EXAMPLES

The following non-limiting examples are provided to illustrate but a few multifunctional polymers and their methods of syntheses.

### Example 1

### Grafting of Poly(Isobutylene-Co-Maleic Anhydride) [Poly(IB-Co-MA)] (M_{w} 6,000 Da) with N-(3-Dimethylaminopropyl)Amine and Ethanol (80% Dimethylaminopropyl Imide, 20% Ethyl Ester; Molar Ratios)

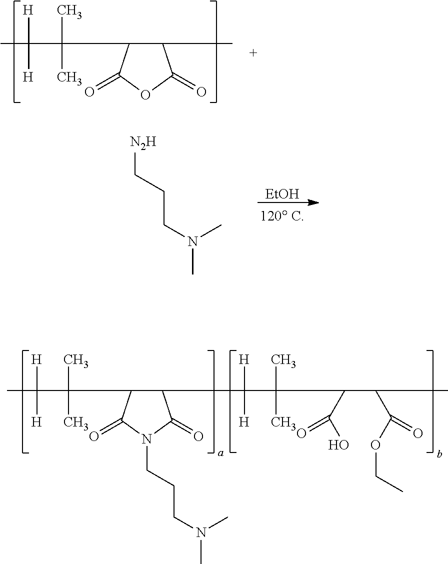

A quantity of 9.97 g of poly(isobutylene-co-maleic anhydride) (poly(IB-co-MA) (Mw 6,000 Da), 5.29 g of N-(3-dimethylaminopropyl)amine (DMAPA), 1.31 g triethylamine, and 30.77 g ethanol were charged into a sealed stainless steel reactor. The mixture was heated at 120° C. for 10 hours. The polymer solution was then cooled and discharged. The ethanol and triethylamine were removed by vacuum stripping and solvent exchange with water. Then, it was neutralized by 1 M HCl solution (1:1 molar of DMAPA). The water was then removed by vacuum stripping and a white powder resulted.

### Example 2

### Grafting of Poly(IB-Co-MA) (M_{w} 6,000 Da) with N-(3-Dimethylaminopropyl)Amine and Ethanol (50% Dimethylaminopropyl Imide, 50% Ethyl Ester; Molar Ratios)

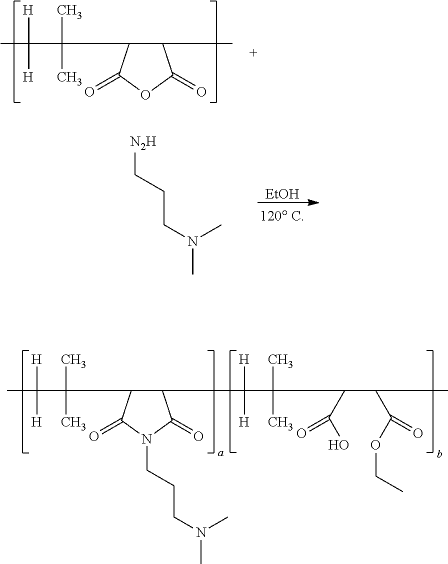

A quantity of 9.98 g of poly(IB-co-MA) (Mw 6,000 Da), 3.31 g of DMAPA, 3.28 g triethylamine, and 30.77 g ethanol were charged into a sealed stainless steel reactor. The mixture was heated at 120° C. for 10 hours. The polymer solution was then cooled and discharged. The ethanol and triethylamine were removed by vacuum stripping and solvent exchange with water. Then, it was neutralized by 1 M HCl solution (1:1 molar of DMAPA). The water was then removed by vacuum stripping and a white powder resulted.

### Example 3

### Grafting of Poly(IB-Co-MA) (M_{w} 6,000 Da) with N-(3-Dimethylaminopropyl)Amine and n-Butylamine, and Ethanol (60% Dimethylaminopropyl Imide, 20% Butylimide, 20% Ethyl Ester; Molar Ratios)

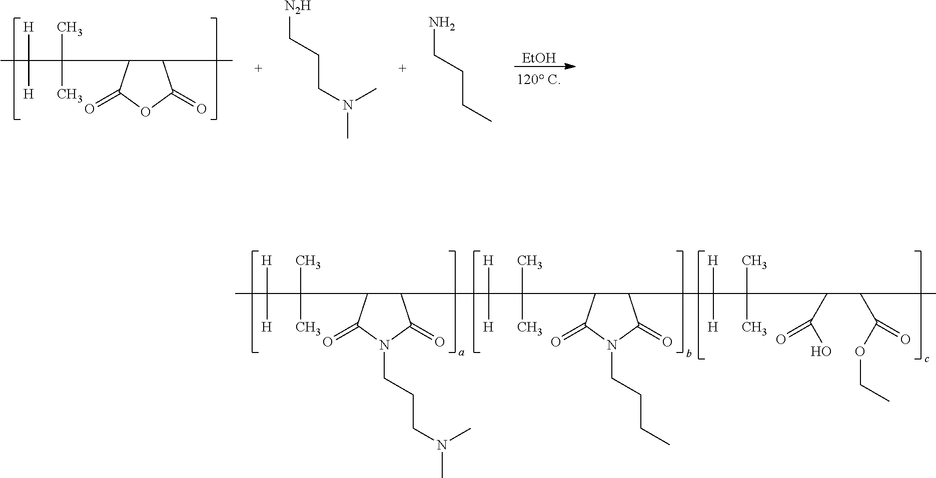

A quantity of 9.97 g of poly(IB-co-MA) (Mw 6,000 Da), 3.96 g of DMAPA, 1.31 g triethylamine, 0.95 g of n-butylamine and 30.77 g ethanol were charged into a sealed stainless steel reactor. The mixture was heated at 120° C. for 10 hours. The polymer solution was then cooled and discharged. The ethanol and triethylamine were removed by vacuum stripping and solvent exchange with water. Then, it was neutralized by 1 M HCl solution (1:1 molar of DMAPA). The water was then removed by vacuum stripping and a white powder resulted.

### Example 4

### Grafting of Poly(IB-Co-MA) (M_{w} 6,000 Da) with N-(3-Dimethylaminopropyl)Amine and n-Butylamine, and Ethanol (60% Dimethylaminopropyl Imide, 20% Butylimide, 20% Ethyl Ester; Molar Ratios)

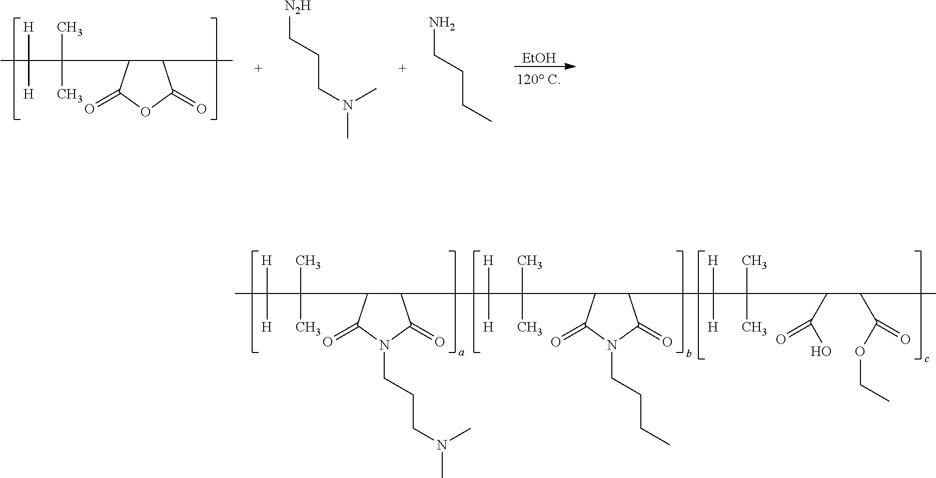

A quantity of 170.06 g of poly(IB-co-MA) (Mw 6,000 Da), 67.63 g of DMAPA, 22.32 g triethylamine, 16.14 g of n-butylamine and 512.85 g ethanol were charged into a sealed stainless steel reactor. The mixture was heated at 120° C. for 10 hours. The polymer solution was then cooled and discharged. The ethanol and triethylamine were removed by vacuum stripping and solvent exchange with water. Then, it was neutralized by 1 M HCl solution (1:1 molar of DMAPA). The water was then removed by vacuum stripping and a white powder resulted.

### Example 5

### Grafting of Poly(IB-Co-MA) (M_{w} 6,000 Da) with N-(3-Dimethylaminopropyl)Amine and n-Butylamine and Ethanol (60% Dimethylaminopropyl Imide, 20% Butylimide, 20% Ethyl Ester; Molar Ratios)

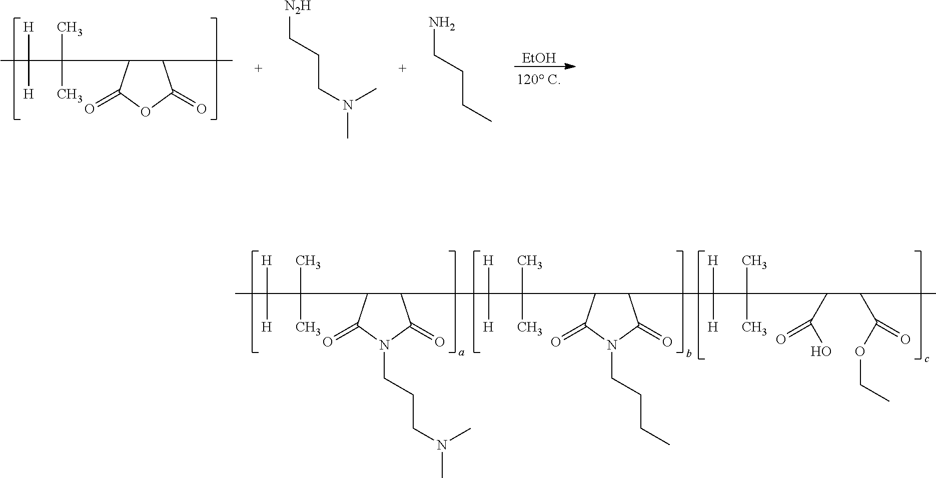

A quantity of 10.22 g of poly(IB-co-MA) (Mw 6,000 Da), 4.07 g DMAPA, 0.97 g n-butylamine, 1.35 g triethylamine and 30.78 g ethanol were added to a 75 mL bomb reactor. Reactor was sealed and placed on a rotation wheel in an oven to react for 10 hours at 120° C. The polymer solution was then cooled and discharged from the reactor. Ethanol and triethylamine were removed by vacuum stripping and the solution was solvent exchanged with water. The solution was then neutralized with 1 M HCl (1:1 molar of DMAPA). Water was then removed by vacuum stripping, resulting in a white powder.

### Example 6

### Grafting of Poly(IB-Co-MA) (M_{w} 6,000 Da) with N-(3-Dimethylaminopropyl)Amine, n-Butylamine, and Ethanol (60% Dimethylaminopropyl Imide, 20% Butylimide, 20% Ethyl Ester; Molar Ratios)

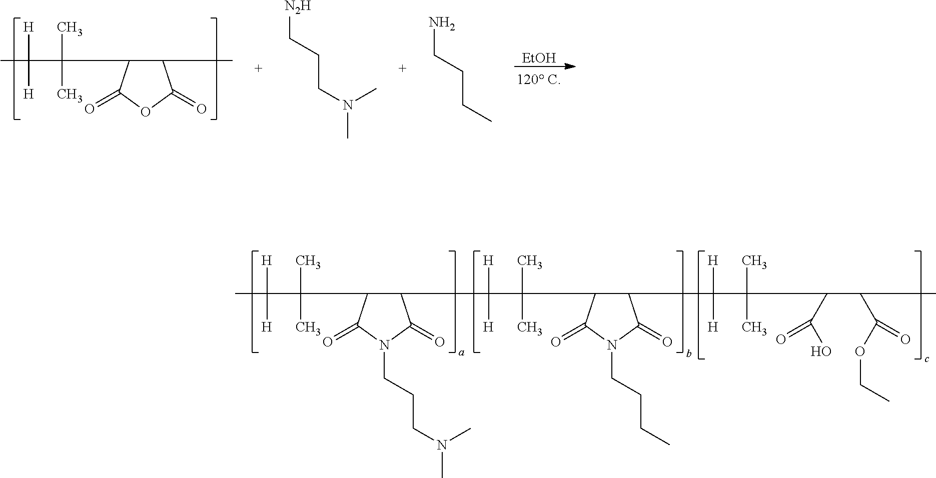

A quantity of 127.54 g of poly(IB-co-MA) (M_{w} 6,000 Da), 50.73 g DMAPA, 12.10 g n-butylamine, 16.74 g triethylamine and 384.64 g ethanol were added to a sealed 1 L stainless steel reactor. The mixture was heated to 120° C. and allowed to react for 10 hours. The polymer solution was then cooled and discharged from the reactor. Ethanol and triethylamine were removed by vacuum stripping and the solution was solvent exchanged with water. The solution was then neutralized with 1 M HCl (1:1 molar of DMAPA). Water was then removed by vacuum stripping, resulting in a white powder.

### Example 7

### Grafting of Poly(IB-Co-MA) (M_{w} 6,000 Da) with N-(3-Dimethylaminopropyl)Amine and n-Butylamine and Ethanol (60% Dimethylaminopropyl Imide, 20% Butylimide, 20% Ethyl Ester; Molar Ratios)

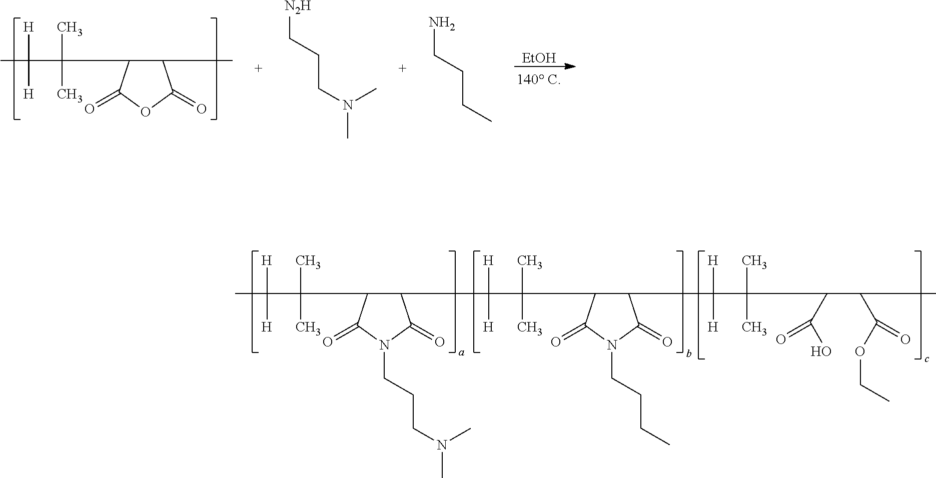

A quantity of 10.21 g of poly(IB-co-MA) (Mw 6,000 Da), 4.06 g DMAPA, 0.97 g n-butylamine, 1.34 g triethylamine and 30.76 g ethanol were added to a 75 mL bomb reactor. Reactor was sealed and placed on a rotation wheel in an oven to react for 10 hours at 140° C. The polymer solution was then cooled and discharged from the reactor. Ethanol and triethylamine were removed by vacuum stripping and the solution was solvent exchanged with water. The solution was then neutralized with 1 M HCl (1:1 molar of DMAPA). Water was then removed by vacuum stripping, resulting in a white powder.

### Example 8

### Grafting of Poly(IB-Co-MA) (M_{w} 6,000 Da) with N-(3-Dimethylaminopropyl)Amine, n-Butylamine, and Ethanol (60% Dimethylaminopropyl Imide, 30% Butylimide, 10% Ethyl Ester; Molar Ratios)

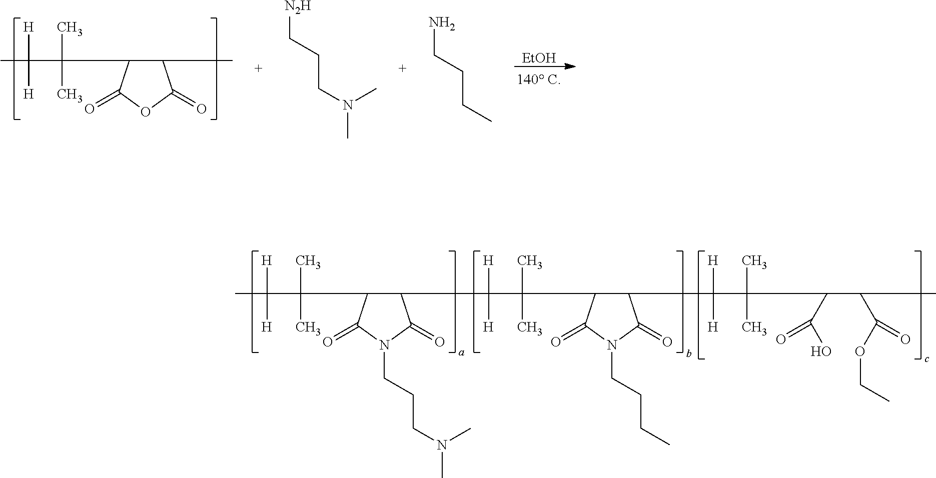

A quantity of 10.31 g of poly(IB-co-MA) (M_{w} 6,000 Da), 4.10 g DMAPA, 1.47 g butylamine, 0.68 g triethylamine and 30.78 g ethanol were added to a 75 mL bomb reactor. Reactor was sealed and placed on a rotation wheel in an oven to react for 10 hours at 140° C. The polymer solution was then cooled and discharged from the reactor. Ethanol and triethylamine were removed by vacuum stripping and the solution was solvent exchanged with water. The solution was then neutralized with 1 M HCl (1:1 molar of DMAPA). Water was then removed by vacuum stripping, resulting in a white powder.

### Example 9

### Grafting of Poly(IB-Co-MA) (M_{w} 6,000 Da) with N-(3-Dimethylaminopropyl)Amine, and n-Butylamine (60% Dimethylaminopropyl Imide, 40% Butylimide, Molar Ratios)

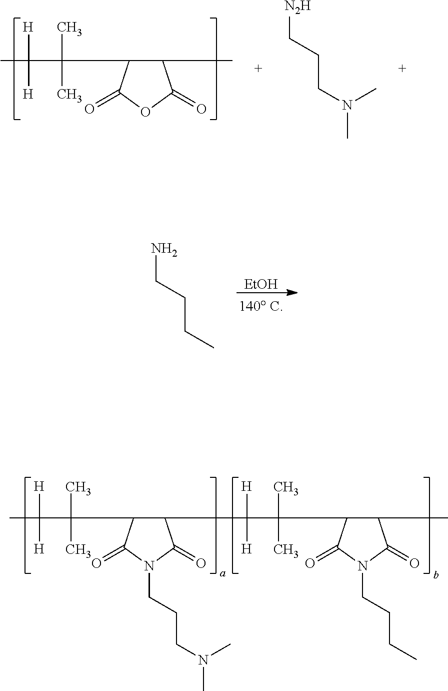

A quantity of 10.45 g of poly(IB-co-MA) (M_{w} 6,000 Da), 4.15 g DMAPA, 1.97 g n-butylamine, and 30.89 g ethanol were added to a 75 mL bomb reactor. Reactor was sealed and placed on a rotation wheel in an oven to react for 10 hours at 140° C. The polymer solution was then cooled and discharged from the reactor. Ethanol was removed by vacuum stripping and the solution was solvent exchanged with water. The solution was then neutralized with 1 M HCl (1:1 molar of DMAPA). Water was then removed by vacuum stripping, resulting in a white powder.

### Example 10

### Grafting of Poly(IB-Co-MA) (M_{w} 6,000 Da) with N-(3-Dimethylaminopropyl)Amine, n-Butylamine, and Ethanol (40% Dimethylaminopropyl Imide, 40% Butylimide, 20% Ethyl Ester; Molar Ratios)

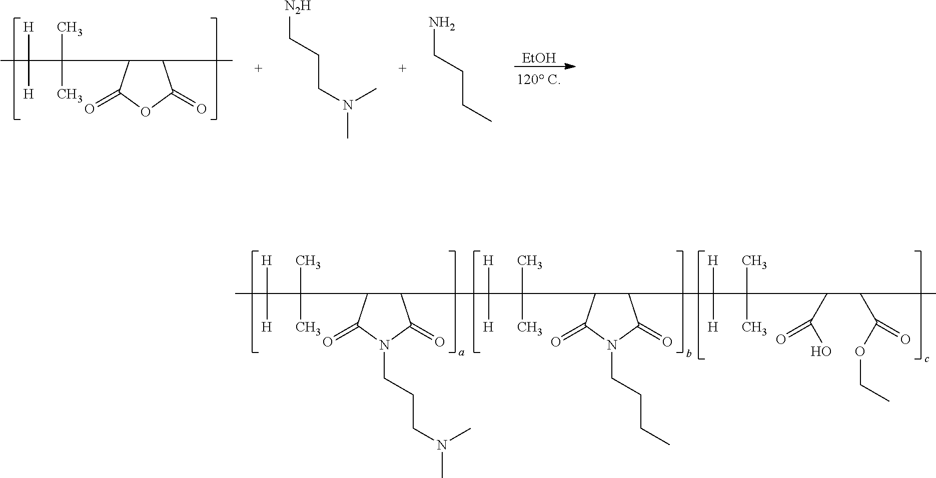

A quantity of 9.97 g of poly(IB-co-MA) (M_{w} 6,000 Da), 2.64 g of DMAPA, 1.31 g triethylamine, 1.89 g of n-butylamine and 30.77 g ethanol were charged into a sealed stainless steel reactor. The mixture was heated at 120° C. for 10 hours. The polymer solution was then cooled and discharged. The ethanol and triethylamine were removed by vacuum stripping and solvent exchange with water. Then, it was neutralized by 1 M HCl solution (1:1 molar of DMAPA). The water was then removed by vacuum stripping and a white powder resulted.

### Example 11

### Grafting of Poly(IB-Co-MA) (M_{w} 80,000 Da) with N-(3-Dimethylaminopropyl)Amine, and Ethanol (80% Dimethylaminopropyl Imide, 20% Ethyl Ester; Molar Ratios)

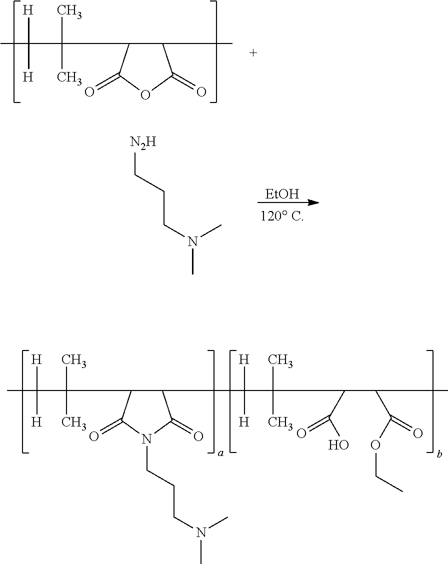

A quantity of 10.14 g of poly(IB-co-MA) (M_{w} 80,000 Da), 5.29 g of DMAPA, 1.31 g triethylamine, and 30.77 g ethanol were charged into a sealed stainless steel reactor. The mixture was heated at 120° C. for 10 hours. The polymer solution was then cooled and discharged. The ethanol and triethylamine were removed by vacuum stripping and solvent exchange with water. Then, it was neutralized by 1 M HCl solution (1:1 molar of DMAPA). The water was then removed by vacuum stripping and a white powder resulted.

### Example 12

### Grafting of Poly(IB-Co-MA) (M_{w} 80,000 Da) with N-(3-Dimethylaminopropyl)Amine and Ethanol (50% Dimethylaminopropyl Imide, 50% Ethyl Ester; Molar Ratios)

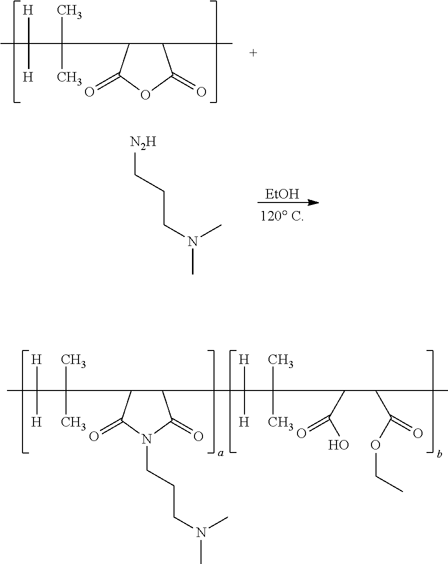

A quantity of 10.14 g of poly(IB-co-MA) (M_{w} 80,000 Da), 3.31 g of DMAPA, 3.28 g triethylamine, and 30.77 g ethanol were charged into a sealed stainless steel reactor. The mixture was heated at 120° C. for 10 hours. The polymer solution was then cooled and discharged. The ethanol and triethylamine were removed by vacuum stripping and solvent exchange with water. Then, it was neutralized by 1 M HCl solution (1:1 molar of DMAPA). The water was then removed by vacuum stripping and a white powder resulted.

### Example 13

### Grafting of Poly(IB-Co-MA) (M_{w} 80,000 Da) with N-(3-Dimethylaminopropyl)Amine and n-Butylamine (60% Dimethylaminopropyl Imide, 40% Butylimide, Molar Ratios)

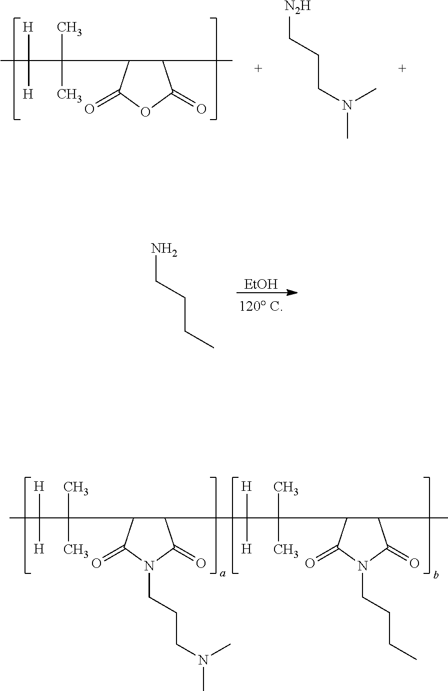

A quantity of 10.14 g of poly(IB-co-MA) (M_{w} 80,000 Da), 3.96 g of DMAPA, 1.31 g triethylamine, 0.95 g of n-butylamine and 30.77 g ethanol were charged into a sealed stainless steel reactor. The mixture was heated at 120° C. for 10 hours. The polymer solution was then cooled and discharged. The ethanol and triethylamine were removed by vacuum stripping and solvent exchange with water. Then, it was neutralized by 1 M HCl solution (1:1 molar of DMAPA). The water was then removed by vacuum stripping and a white powder resulted.

### Example 14

### Grafting of Poly(IB-Co-MA) (M_{w} 80,000 Da) with N-(3-Dimethylaminopropyl)Amine and n-Butylamine (60% Dimethylaminopropyl Imide, 40% Butylimide, Molar Ratios)

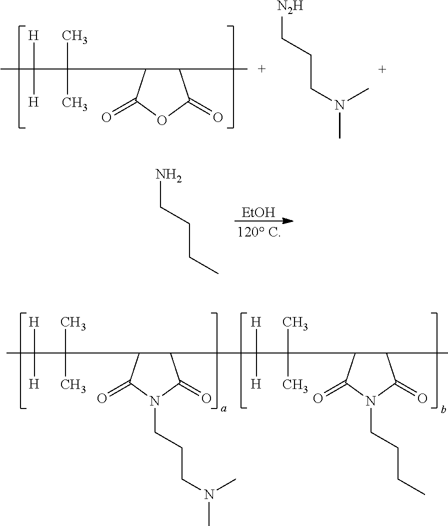

A quantity of 170.06 g of poly(IB-co-MA) (M_{w} 80,000 Da), 67.63 g of DMAPA, 22.32 g triethylamine, 16.14 g of n-butylamine and 512.85 g ethanol were charged into a sealed stainless steel reactor. The mixture was heated at 120° C. for 10 hours. The polymer solution was then cooled and discharged. The ethanol and triethylamine were removed by vacuum stripping and solvent exchange with water. Then, it was neutralized by 1 M HCl solution (1:1 molar of DMAPA). The water was then removed by vacuum stripping and a white powder resulted.

### Example 15

### Grafting of Poly(IB-Co-MA) (M_{w} 80,000 Da) with N-(3-Dimethylaminopropyl)Amine and n-Butylamine, and Ethanol (60% Dimethylaminopropyl Imide, 20% Butylimide, 20% Ethyl Ester; Molar Ratios)

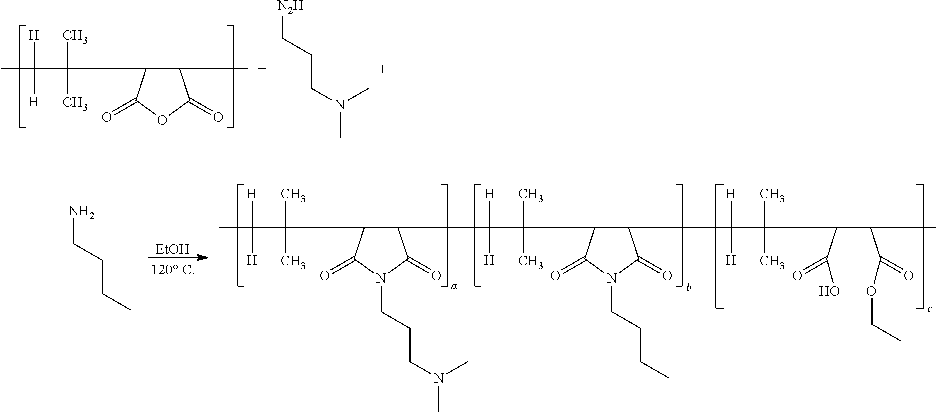

A quantity of 10.19 g of poly(IB-co-MA) (M_{w} 80,000 Da), 4.06 g DMAPA, 0.97 g n-butylamine, 1.34 g triethylamine and 30.78 g ethanol were added to a 75 mL bomb reactor. Reactor was sealed and placed on a rotation wheel in an oven to react for 10 hours at 120° C. The polymer solution was then cooled and discharged from the reactor. Ethanol and triethylamine were removed by vacuum stripping and the solution was solvent exchanged with water. The solution was then neutralized with 1 M HCl (1:1 molar of DMAPA). Water was then removed by vacuum stripping, resulting in a white powder.

### Example 16

### Grafting of Poly(IB-Co-MA) (M_{w} 80,000 Da) with N-(3-Dimethylaminopropyl)Amine and n-Butylamine, and Ethanol (60% Dimethylaminopropyl Imide, 20% Butylimide, 20% Ethyl Ester; Molar Ratios)

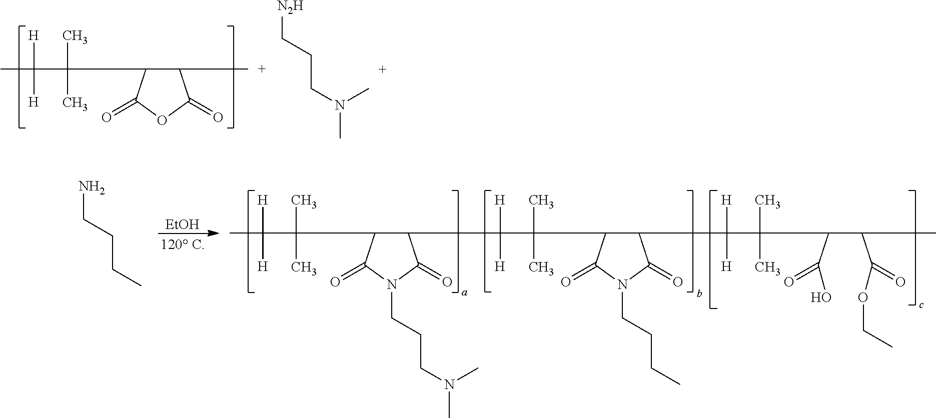

A quantity of 127.53 g of poly(IB-co-MA) (M_{w} 80,000 Da), 50.73 g DMAPA, 12.08 g n-butylamine, 16.75 g triethylamine and 384.69 g ethanol were added to a sealed 1 L stainless steel reactor. The mixture was heated to 120° C. and allowed to react for 10 hours. The polymer solution was then cooled and discharged from the reactor. Ethanol and triethylamine were removed by vacuum stripping and the solution was solvent exchanged with water. The solution was then neutralized with 1 M HCl (1:1 molar of DMAPA). Water was then removed by vacuum stripping, resulting in a white powder.

### Example 17

### Grafting of Poly(IB-Co-MA) (M_{w} 80,000 Da) with N-(3-Dimethylaminopropyl)Amine and n-Butylamine, and Ethanol (40% Dimethylaminopropyl Imide, 40% Butylimide, 20% Ethyl Ester; Molar Ratios)

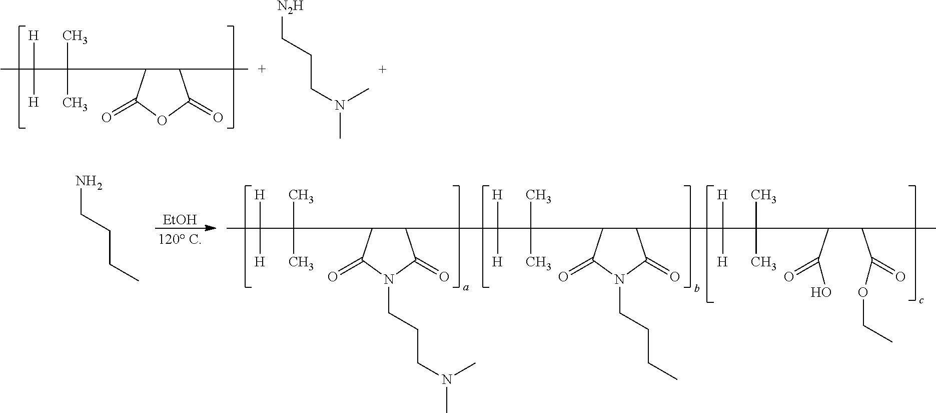

A quantity of 10.14 g of poly(IB-co-MA) (M_{w} 80,000 Da), 2.64 g of DMAPA, 1.31 g triethylamine, 1.89 g of n-butylamine and 30.77 g ethanol were charged into a sealed stainless steel reactor. The mixture was heated at 120° C. for 10 hours. The polymer solution was then cooled and discharged. The ethanol and triethylamine were removed by vacuum stripping and solvent exchange with water. Then, it was neutralized by 1 M HCl solution (1:1 molar of DMAPA). The water was then removed by vacuum stripping and a white powder resulted.

### Example 18

### Grafting of Poly(IB-Co-MA) (M_{w} 240,000 Da) with N-(3-Dimethylaminopropyl)Amine, and Ethanol (50% Dimethylaminopropyl Imide, 50% Ethyl Ester; Molar Ratios)

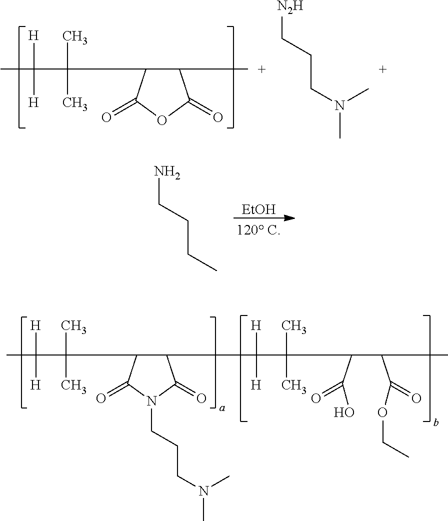

A quantity of 9.97 g of poly(IB-co-MA) (M_{w} 240,000 Da), 5.29 g of DMAPA, 1.31 g triethylamine, and 30.77 g ethanol were charged into a sealed stainless steel reactor. The mixture was heated at 120° C. for 10 hours. The polymer solution was then cooled and discharged. The ethanol and triethylamine were removed by vacuum stripping and solvent exchange with water. Then, it was neutralized by 1 M HCl solution (1:1 molar of DMAPA). The water was then removed by vacuum stripping and a white powder resulted.

### Example 19

### Grafting of Poly(IB-Co-MA) (M_{w} 240,000 Da) with N-(3-Dimethylaminopropyl)Amine, and Ethanol (50% Dimethylaminopropyl Imide, 50% Ethyl Ester; Molar Ratios)

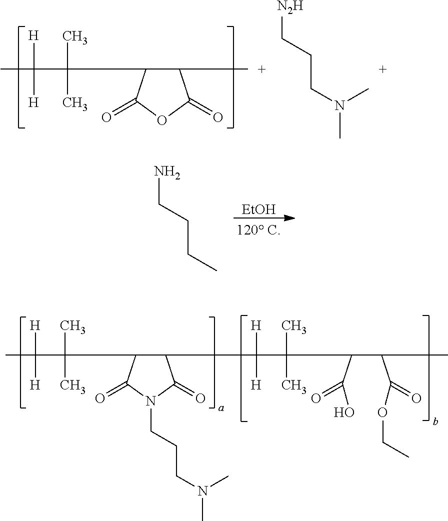

A quantity of 9.98 g of poly(IB-co-MA) (M_{w} 240,000 Da), 3.31 g of DMAPA, 3.28 g triethylamine, and 30.77 g ethanol were charged into a sealed stainless steel reactor. The mixture was heated at 120° C. for 10 hours. The polymer solution was then cooled and discharged. The ethanol and triethylamine were removed by vacuum stripping and solvent exchange with water. Then, it was neutralized by 1 M HCl solution (1:1 molar of DMAPA). The water was then removed by vacuum stripping and a white powder resulted.

### Example 20

### Grafting of Poly(IB-Co-MA), M_{w} 240,000 Da) with N-(3-Dimethylaminopropyl)Amine, Ethanol, and Butylamine (60% Dimethylaminopropyl Imide, 20% Butylimide, 20% Ethyl Ester; Molar Ratios)

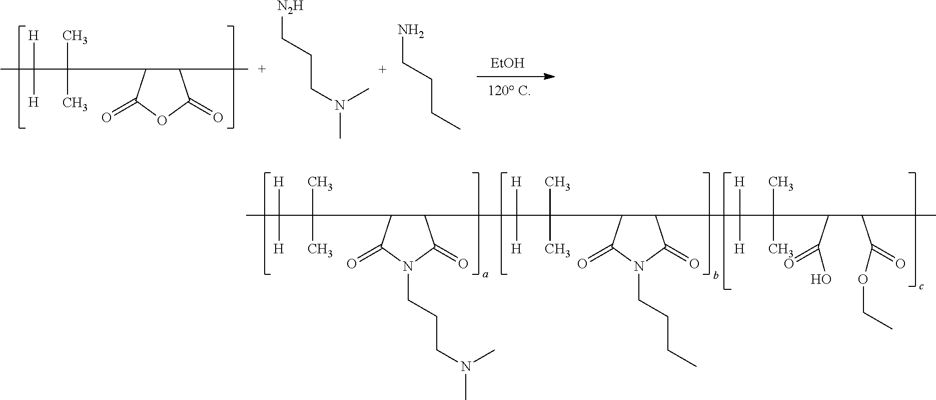

A quantity of 9.97 g of poly(IB-co-MA) (M_{w} 240,000 Da), 3.96 g of DMAPA, 1.31 g triethylamine, 0.95 g of butylamine and 30.77 g ethanol were charged into a sealed stainless steel reactor. The mixture was heated at 120° C. for 10 hours. The polymer solution was then cooled and discharged. The ethanol and triethylamine were removed by vacuum stripping and solvent exchange with water. Then, it was neutralized by 1 M HCl solution (1:1 molar of DMAPA). The water was then removed by vacuum stripping and a white powder resulted.

### Example 21

### Grafting of Poly(IB-Co-MA) (M_{w} 240,000 Da) with N-(3-Dimethylaminopropyl)Amine and n-Butylamine, and Ethanol (40% Dimethylaminopropyl Imide, 40% Butylimide, 20% Ethyl Ester; Molar Ratios)

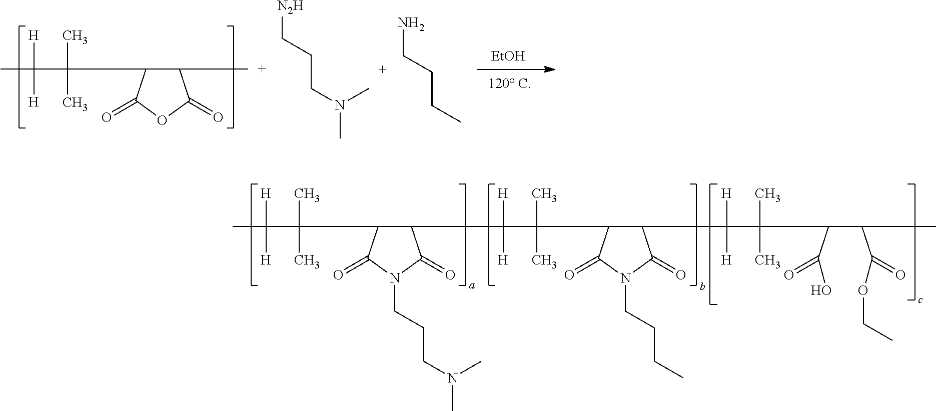

A quantity of 9.97 g of poly(IB-co-MA) (M_{w} 240,000 Da), 2.64 g of DMAPA, 1.31 g triethylamine, 1.89 g of n-butylamine and 30.77 g ethanol were charged into a sealed stainless steel reactor. The mixture was heated at 120° C. for 10 hours. The polymer solution was then cooled and discharged. The ethanol and triethylamine were removed by vacuum stripping and solvent exchange with water. Then, it was neutralized by 1 M HCl solution (1:1 molar of DMAPA). The water was then removed by vacuum stripping and a white powder resulted.

### Example 22

### Grafting of Poly(IB-Co-MA) (M_{w} 6,000 Da) with Dopamine, and Ethanol (50% Dopamine Imide, 50% Ethyl Ester; Molar Ratios)

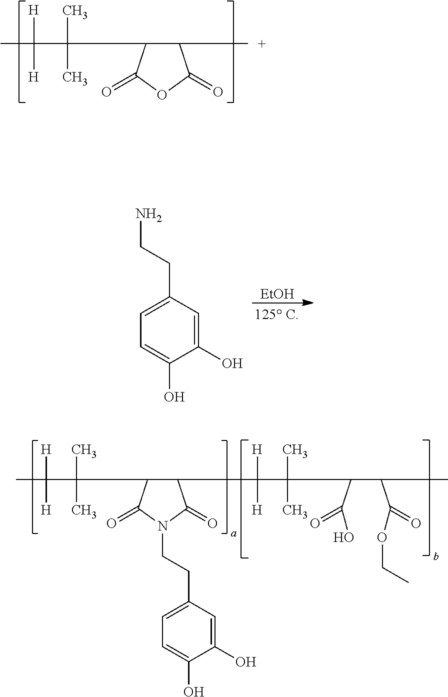

A quantity of 9.08 g of poly(IB-co-MA) (M_{w} 6,000 Da), 4.51 g of dopamine(4-(2-aminoethyl)benzene-1,2-diol), 2.98 g triethylamine, and 30.77 g ethanol were charged into a sealed stainless steel reactor. The mixture was heated at 125° C. for 10 hours. The polymer solution was then cooled and discharged. The ethanol and triethylamine were removed by vacuum stripping and solvent exchange with water. Then, it was neutralized by 1 M NaOH solution. The water was then removed by vacuum stripping and a powder resulted.

### Example 23

### Grafting of Poly(IB-Co-MA) (M_{w} 80,000 Da) with Dopamine, and Ethanol (50% Dopamine Imide, 50% Ethyl Ester; Molar Ratios)

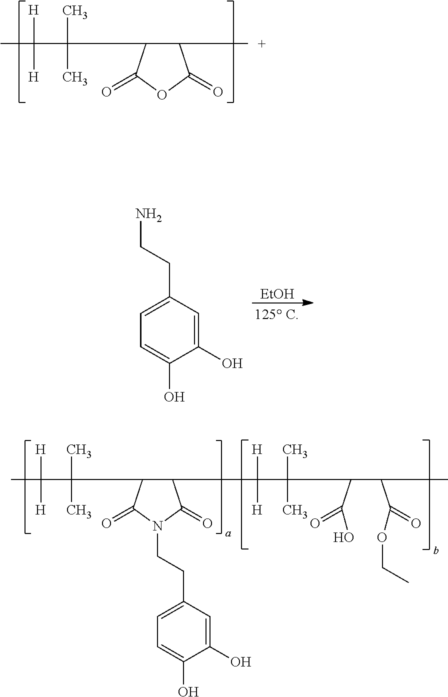

A quantity of 9.08 g of poly(IB-co-MA) (M_{w} 80,000 Da), 4.51 g of dopamine, 2.98 g triethylamine, and 30.77 g ethanol were charged into a sealed stainless steel reactor. The mixture was heated at 125° C. for 10 hours. The polymer solution was then cooled and discharged. The ethanol and triethylamine were removed by vacuum stripping and solvent exchange with water. Then, it was neutralized by 1 M NaOH solution. The water was then removed by vacuum stripping and a powder resulted.

### Example 24

### Grafting of Poly(IB-Co-MA) (M_{w} 240,000 Da) with Dopamine, and Ethanol (50% Dopamine Imide, 50% Ethyl Ester; Molar Ratios)

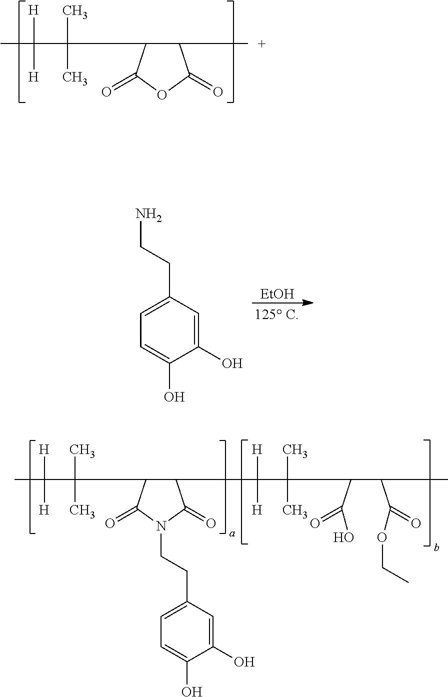

A quantity of 9.08 g of poly(IB-co-MA) (M_{w} 240,000 Da), 4.51 g of dopamine, 2.98 g triethylamine, and 30.77 g ethanol were charged into a sealed stainless steel reactor. The mixture was heated at 125° C. for 10 hours. The polymer solution was then cooled and discharged. The ethanol and triethylamine were removed by vacuum stripping and solvent exchange with water. Then, it was neutralized by 1 M NaOH solution. The water was then removed by vacuum stripping and a powder resulted.

### Example 25

### Grafting of Poly(Isobutylene-Co-Maleic Anhydride) (Poly(IB-Co-MA), M_{w} 6,000 Da) with Aminopropyl Imidazole, and Ethanol (50% Aminopropyl Imidazole Imide, 50% Ethyl Ester; Molar Ratios)

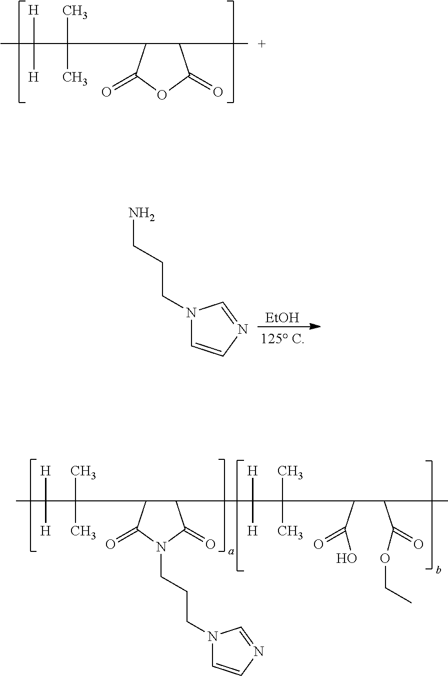

A quantity of 9.55 g of poly(IB-co-MA) (M_{w} 6,000 Da), 3.88 g of aminopropyl imidazole, 3.14 g triethylamine, and 30.77 g ethanol were charged into a sealed stainless steel reactor. The mixture was heated at 125° C. for 10 hours. The polymer solution was then cooled and discharged. The ethanol and triethylamine were removed by vacuum stripping and solvent exchange with water. Then, it was neutralized by 1 M HCl solution. The water was then removed by vacuum stripping and a powder resulted.

### Example 26

### Grafting of Poly(Isobutylene-Co-Maleic Anhydride) (Poly(IB-Co-MA), M_{w} 6,000 Da) with Aminopropyl Imidazole and n-Butylamine, Ethyl Ester (40% Aminopropyl Imidazole Imide, 40% Butylimide, 20% Ethyl Ester; Molar Ratios)

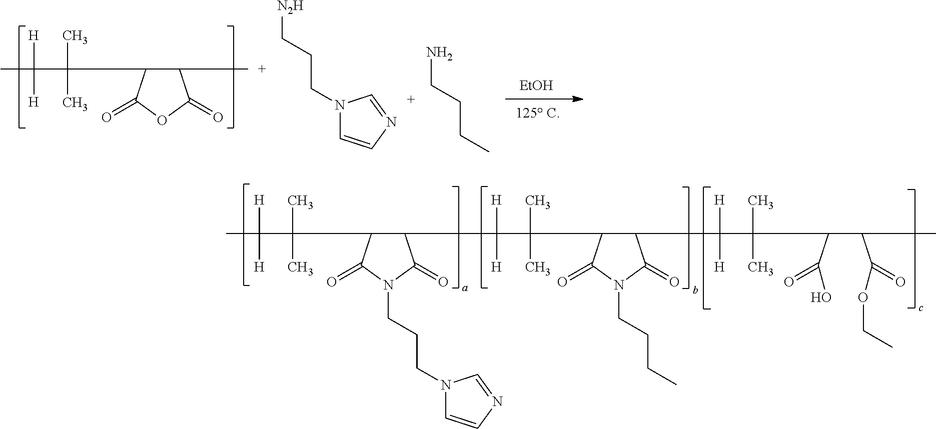

A quantity of 10.07 g of poly(IB-co-MA) (M_{w} 6,000 Da), 3.27 g of aminopropyl imidazole, 1.32 g triethylamine, and 30.77 g ethanol were charged into a sealed stainless steel reactor. The mixture was heated at 125° C. for 10 hours. The polymer solution was then cooled and discharged. The ethanol and triethylamine were removed by vacuum stripping and solvent exchange with water. Then, it was neutralized by 1 M HCl solution. The water was then removed by vacuum stripping and a powder resulted.

### Example 27

### Grafting of Poly(IB-Co-MA), M_{w} 6,000 Da) with Aminopropyl Imidazole, Ethanol, and Butylamine (60% Aminopropyl Imidazole Imide, 20% Butylamide, 20% Ethyl Ester; Molar Ratios)

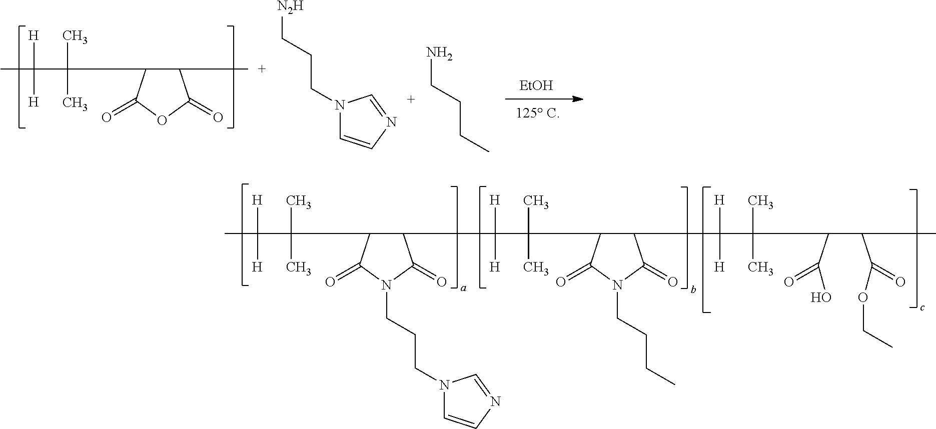

A quantity of 9.67 g of poly(IB-co-MA) (M_{w} 6,000 Da), 4.71 g of aminopropyl imidazole, 1.27 g triethylamine, 0.92 g of butylamine and 30.77 g ethanol were charged into a sealed stainless steel reactor. The mixture was heated at 125° C. for 10 hours. The polymer solution was then cooled and discharged. The ethanol and triethylamine were removed by vacuum stripping and solvent exchange with water. Then, it was neutralized by 1 M HCl solution. The water was then removed by vacuum stripping and a powder resulted.

### Example 28

### Grafting of Poly(Isobutylene-Co-Maleic Anhydride) (Poly(IB-Co-MA), M_{w} 80,000 Da) with Aminopropyl Imidazole and Ethanol (50% Aminopropylimidazole Imide, 50% Ethyl Ester; Molar Ratios)

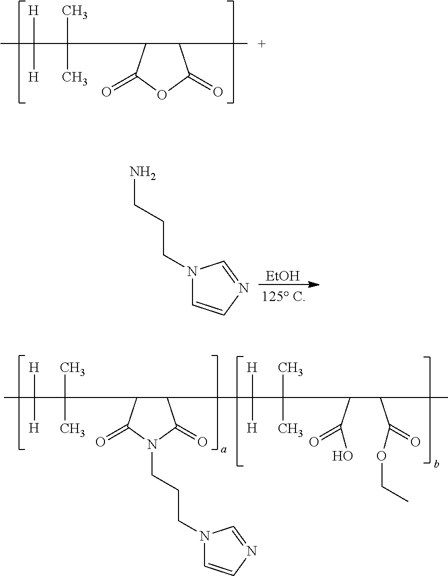

A quantity of 9.55 g of poly(IB-co-MA) (M_{w} 80,000 Da), 3.88 g of aminopropyl imidazole, 3.14 g triethylamine, and 30.77 g ethanol were charged into a sealed stainless steel reactor. The mixture was heated at 125° C. for 10 hours. The polymer solution was then cooled and discharged. The ethanol and triethylamine were removed by vacuum stripping and solvent exchange with water. Then, it was neutralized by 1 M HCl solution. The water was then removed by vacuum stripping and a powder resulted.

### Example 29

### Grafting of Poly(Isobutylene-Co-Maleic Anhydride) (Poly(IB-Co-MA), M_{w} 80,000 Da) with Aminopropyl Imidazole, Ethanol, and Butylamine (40% Aminopropyl Imidazole Imide, 40% Butylimide, 20% Ethyl Ester; Molar Ratios)

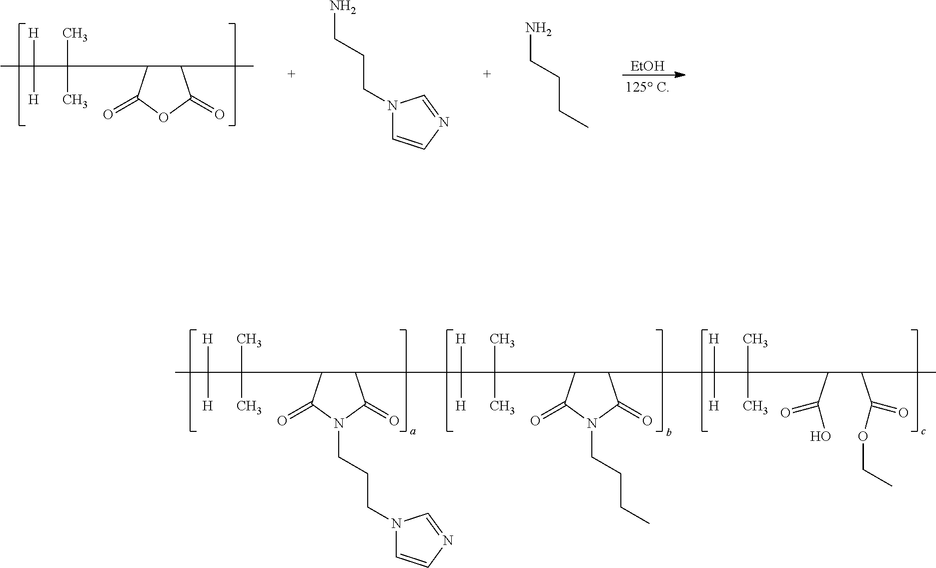

10.07 g of poly(IB-co-MA) (M_{w} 80,000 Da), 3.27 g of aminopropyl imidazole, 1.32 g triethylamine, and 30.77 g ethanol were charged into a sealed stainless steel reactor. The mixture was heated at 125° C. for 10 hours. The polymer solution was then cooled and discharged. The ethanol and triethylamine were removed by vacuum stripping and solvent exchange with water. Then, it was neutralized by 1 M HCl solution. The water was then removed by vacuum stripping and a powder resulted.

### Example 30

### Grafting of Poly(IB-Co-MA) (M_{w} 80,000 Da) with Aminopropyl Imidazole, Ethanol, and Butylamine (60% Aminopropyl Imidazole Imide, 20% Butylimide, and 20% Ethyl Ester; Molar Ratios)

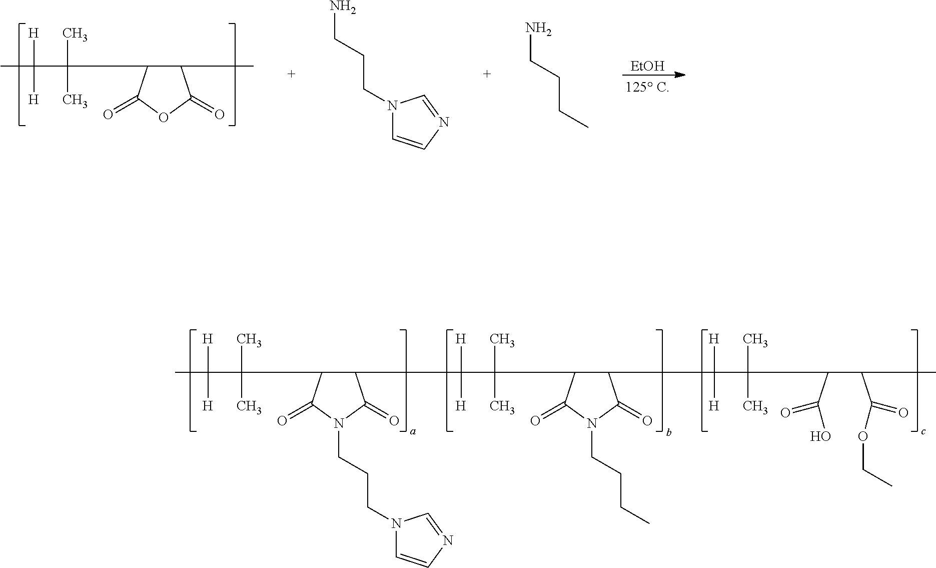

A quantity of 9.67 g of poly(IB-co-MA) (M_{w} 80,000 Da), 4.71 g of aminopropyl imidazole, 1.27 g triethylamine, 0.92 g of butylamine and 30.77 g ethanol were charged into a sealed stainless steel reactor. The mixture was heated at 125° C. for 10 hours. The polymer solution was then cooled and discharged. The ethanol and triethylamine were removed by vacuum stripping and solvent exchange with water. Then, it was neutralized by 1 M HCl solution. The water was then removed by vacuum stripping and a powder resulted.

### Example 31

### Grafting of Poly(IB-Co-MA), M_{w} 240,000 Da) with Aminopropyl Imidazole and Ethanol (50% Aminopropylimidiazole Imide, 50% Ethyl Ester; Molar Ratios)

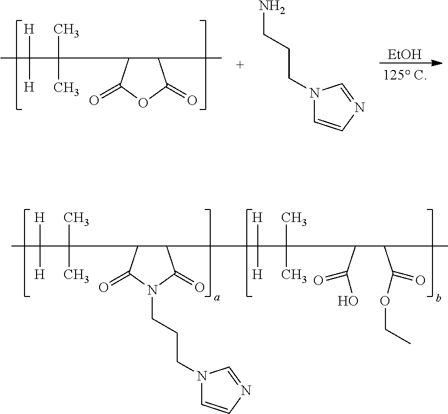

A quantity of 9.55 g of poly(IB-co-MA) (M_{w} 240,000 Da), 3.88 g of aminopropyl imidazole, 3.14 g triethylamine, and 30.77 g ethanol were charged into a sealed stainless steel reactor. The mixture was heated at 125° C. for 10 hours. The polymer solution was then cooled and discharged. The ethanol and triethylamine were removed by vacuum stripping and solvent exchange with water. Then, it was neutralized by 1 M HCl solution. The water was then removed by vacuum stripping and a powder resulted.

### Example 32

### Grafting of Poly(IB-Co-MA) (M_{w} 240,000 Da) with Aminopropyl Imidazole, Ethanol, and Butylamine (40% Aminopropyl Imidazole Imide, 40% Butylimide, 20% Ethyl Ester; Molar Ratios)

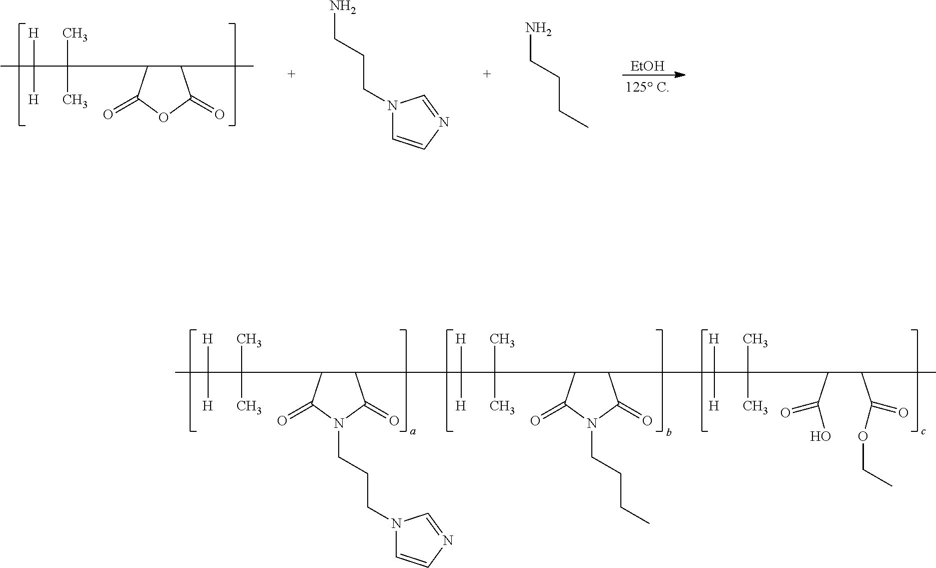

A quantity of 10.07 g of poly(IB-co-MA) (M_{w} 240,000 Da), 3.27 g of aminopropyl imidazole, 1.32 g triethylamine, and 30.77 g ethanol were charged into a sealed stainless steel reactor. The mixture was heated at 125° C. for 10 hours. The polymer solution was then cooled and discharged. The ethanol and triethylamine were removed by vacuum stripping and solvent exchange with water. Then, it was neutralized by 1 M HCl solution. The water was then removed by vacuum stripping and a powder resulted.

### Example 33

### Grafting of Poly(IB-Co-MA) (M_{w} 240,000 Da) with Aminopropyl Imidazole, n-Butylamine, and Ethanol (60% Aminopropyl Imidazole Imide, 20% Butylimide, 20% Ethyl Ester; Molar Ratios)

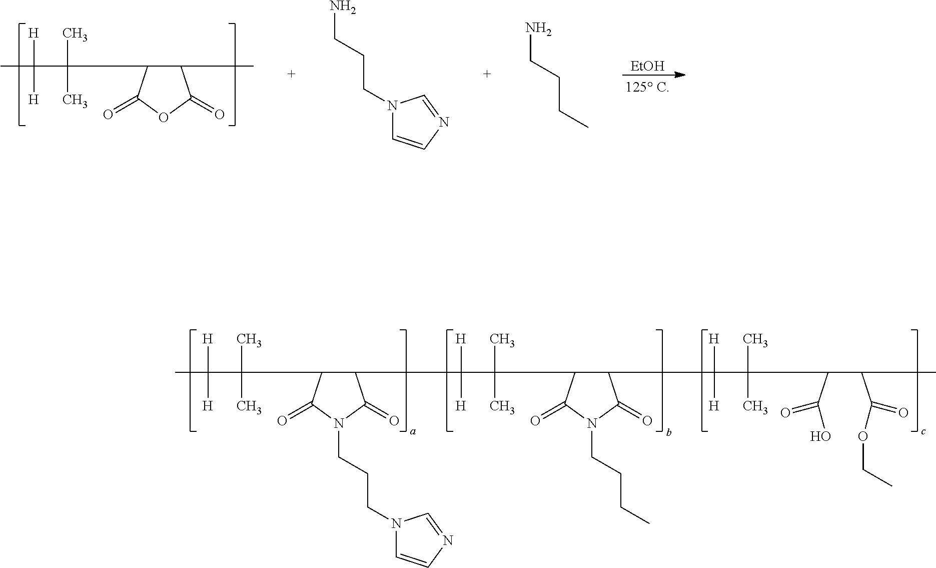

A quantity of 9.67 g of poly(IB-co-MA) (M_{w} 240,000 Da), 4.71 g of aminopropyl imidazole, 1.27 g triethylamine, 0.92 g of butylamine and 30.77 g ethanol were charged into a sealed stainless steel reactor. The mixture was heated at 125° C. for 10 hours. The polymer solution was then cooled and discharged. The ethanol and triethylamine were removed by vacuum stripping and solvent exchange with water. Then, it was neutralized by 1 M HCl solution. The water was then removed by vacuum stripping and a powder resulted.

### Example 34

### Grafting of Poly(Isobutylene-Co-Maleic Anhydride) (Poly(IB-Co-MA), M_{w} 6,000 Da) with p-Aminophenol and Ethanol (50% p-Aminophenol Imide, 50% Ethyl Ester; Molar Ratios)

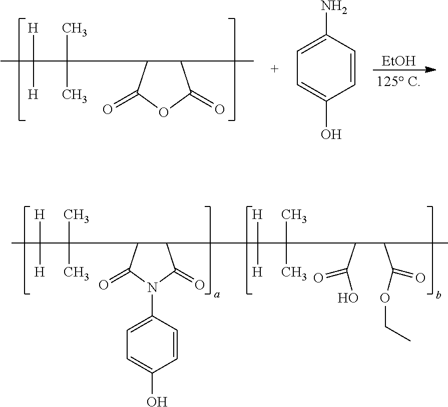

A quantity of 9.11 g of poly(IB-co-MA) (M_{w} 6,000 Da), 4.47 g of p-aminophenol, 2.99 g triethylamine, and 30.77 g ethanol were charged into a sealed stainless steel reactor. The mixture was heated at 125° C. for 10 hours. The polymer solution was then cooled and discharged. The ethanol and triethylamine were removed by vacuum stripping and solvent exchange with water. Then, it was neutralized by 1 M NaOH solution. The water was then removed by vacuum stripping and a powder resulted.

### Example 35

### Grafting of Poly(Isobutylene-Co-Maleic Anhydride) (Poly(IB-Co-MA), M_{w} 80,000 Da) with p-Aminophenol and Ethanol (50% p-Aminophenol Imide, 50% Ethyl Ester; Molar Ratios)

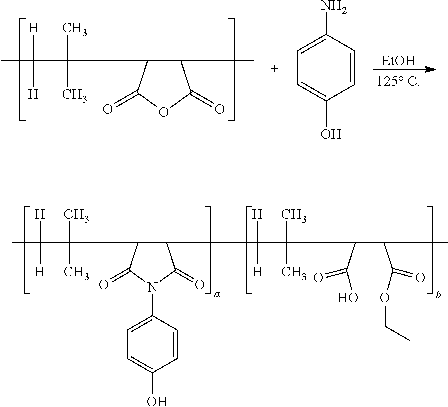

A quantity of 9.11 g of poly(IB-co-MA) (M_{w} 80,000 Da), 4.47 g of p-aminophenol, 2.99 g triethylamine, and 30.77 g ethanol were charged into a sealed stainless steel reactor. The mixture was heated at 125° C. for 10 hours. The polymer solution was then cooled and discharged. The ethanol and triethylamine were removed by vacuum stripping and solvent exchange with water. Then, it was neutralized by 1 M NaOH solution. The water was then removed by vacuum stripping and a powder resulted.

### Example 36

### Of Poly(Isobutylene-Co-Maleic Anhydride) (Poly(IB-Co-MA), M_{w} 240,000 Da) with p-Aminophenol and Ethanol (50% p-Aminophenol Imide, 50% Ethyl Ester; Molar Ratios)

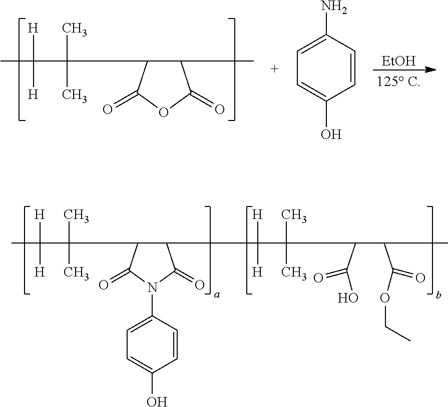

A quantity of 9.11 g of poly(IB-co-MA) (M_{w} 240,000 Da), 4.47 g of p-aminophenol, 2.99 g triethylamine, and 30.77 g ethanol were charged into a sealed stainless steel reactor. The mixture was heated at 125° C. for 10 hours. The polymer solution was then cooled and discharged. The ethanol and triethylamine were removed by vacuum stripping and solvent exchange with water. Then, it was neutralized by 1 M NaOH solution. The water was then removed by vacuum stripping and a powder resulted.

### Example 37

### Grafting of Poly(IB-Co-MA) (M_{w} 6,000 Da) with N-(3-Dimethylaminopropyl)Amine, n-Octylamine, and Ethanol (60% Dimethylaminopropyl Imide, 20% n-Octylimide, 20% Ethyl Ester; Molar Ratios)

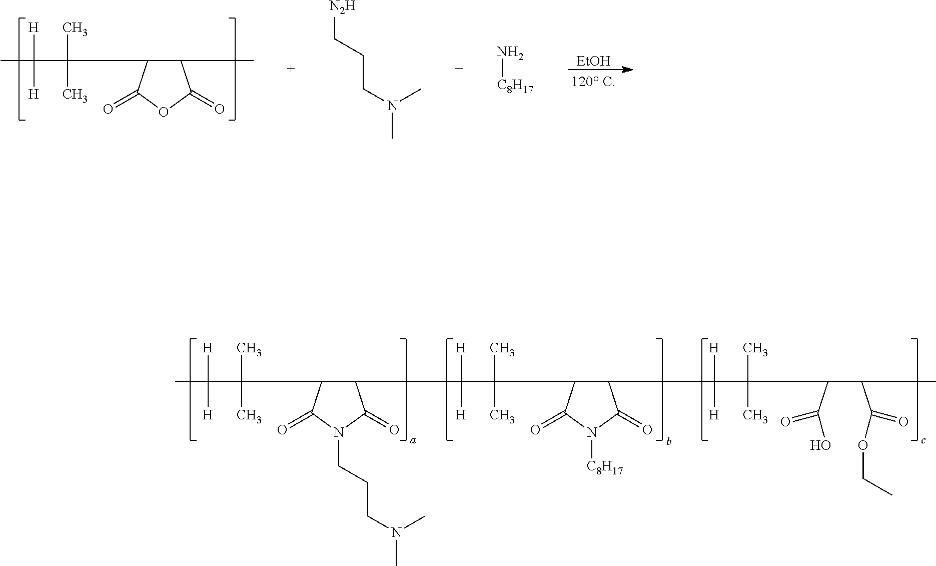

A quantity of 9.77 g of poly(IB-co-MA) (M_{w} 6,000 Da), 3.88 g of DMAPA, 1.28 g triethylamine, 1.64 g of octylamine and 30.77 g ethanol were charged into a sealed stainless steel reactor. The mixture was heated at 120° C. for 10 hours. The polymer solution was then cooled and discharged. The ethanol and triethylamine were removed by vacuum stripping and solvent exchange with water. Then, it was neutralized by 1 M HCl solution (1:1 molar of DMAPA). The water was then removed by vacuum stripping and a white powder resulted.

### Example 38

### Grafting of Poly(IB-Co-MA) (M_{w} 80,000 Da) with N-(3-Dimethylaminopropyl)Amine, n-Octylamine, and Ethanol (60% Dimethylaminopropyl Imide, 20% n-Octylimide, 20% Ethyl Ester; Molar Ratios)

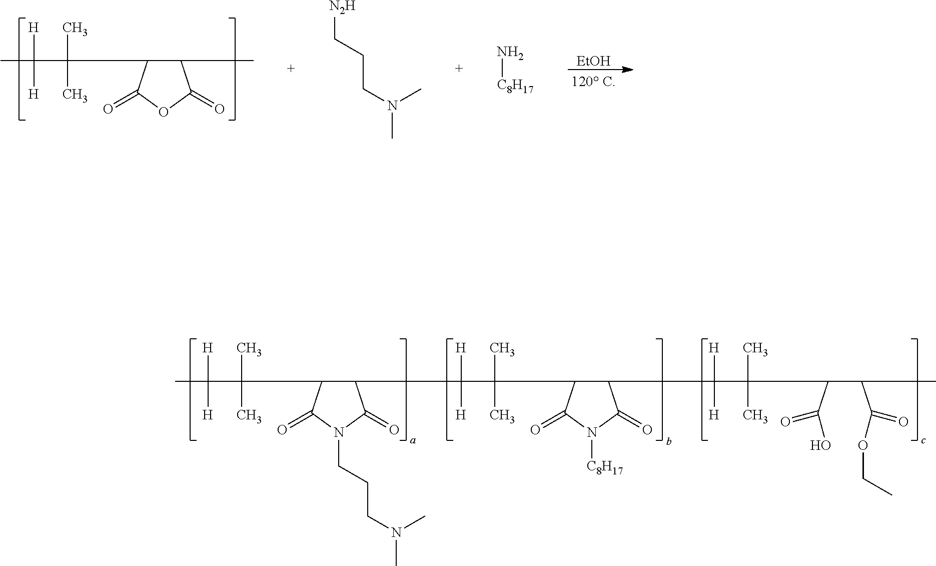

A quantity of 9.77 g of poly(IB-co-MA) (Mw 80,000 Da), 3.88 g of DMAPA, 1.28 g triethylamine, 1.64 g of octylamine and 30.77 g ethanol were charged into a sealed stainless steel reactor. The mixture was heated at 120° C. for 10 hours. The polymer solution was then cooled and discharged. The ethanol and triethylamine were removed by vacuum stripping and solvent exchange with water. Then, it was neutralized by 1 M HCl solution (1:1 molar of DMAPA). The water was then removed by vacuum stripping and a white powder resulted.

### Example 39

### Grafting of Poly(IB-Co-MA) (M_{w} 240,000 Da) with N-(3-Dimethylaminopropyl)Amine, n-Octylamine, and Ethanol (60% Dimethylaminopropyl Imide, 20% n-Octylimide, 20% Ethyl Ester; Molar Ratios)

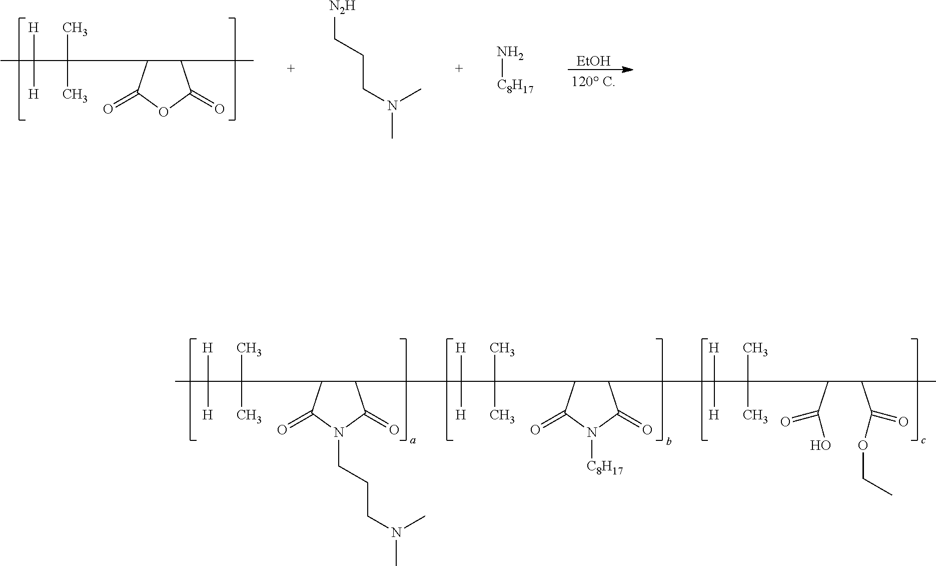

A quantity of 9.77 g of poly(IB-co-MA) (M_{w} 240,000 Da), 3.88 g of DMAPA, 1.28 g triethylamine, 1.64 g of octylamine and 30.77 g ethanol were charged into a sealed stainless steel reactor. The mixture was heated at 120° C. for 10 hours. The polymer solution was then cooled and discharged. The ethanol and triethylamine were removed by vacuum stripping and solvent exchange with water. Then, it was neutralized by 1 M HCl solution (1:1 molar of DMAPA). The water was then removed by vacuum stripping and a white powder resulted.

### Example 40

### Grafting of Poly(IB-Co-MA) (M_{w} 6,000 Da) with N-(3-Dimethylaminopropyl)Amine, Ethanol, and Dodecylamine (60% Dimethylaminopropyl Imide, 20% Dodecylimide, 20% Ethyl Ester; Molar Ratios)

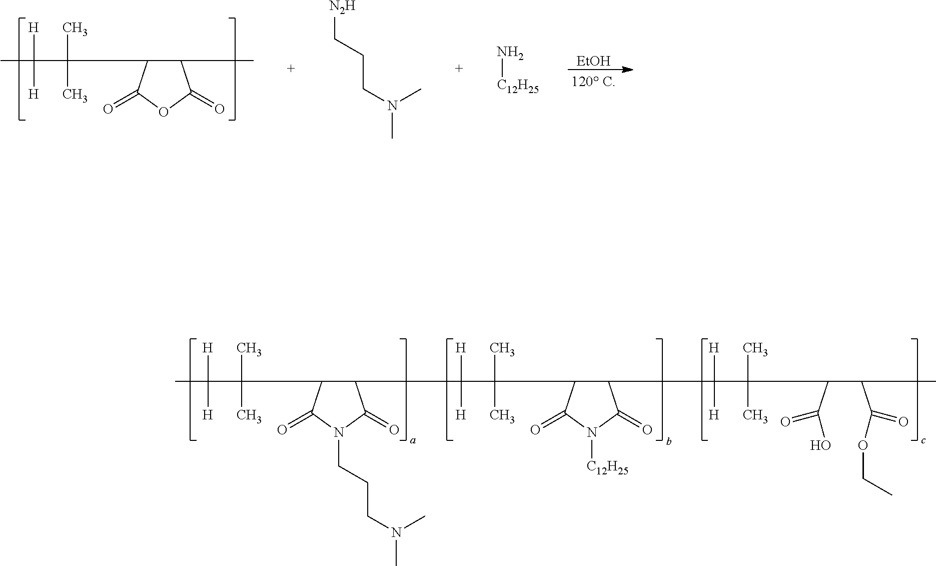

A quantity of 9.36 g of poly(IB-co-MA) (M_{w} 6,000 Da), 3.72 g of DMAPA, 1.23 g triethylamine, 2.25 g of dodecylamine and 30.77 g ethanol were charged into a sealed stainless steel reactor. The mixture was heated at 120° C. for 10 hours. The polymer solution was then cooled and discharged. The ethanol and triethylamine were removed by vacuum stripping and solvent exchange with water. Then, it was neutralized by 1 M HCl solution (1:1 molar of DMAPA). The water was then removed by vacuum stripping and a white powder resulted.

### Example 41

### Grafting of Poly(IB-Co-MA) (M_{w} 80,000 Da) with N-(3-Dimethylaminopropyl)Amine, Ethanol, and Dodecylamine (60% Dimethylaminopropyl Imide, 20% Dodecylimide, 20% Ethyl Ester; Molar Ratios)

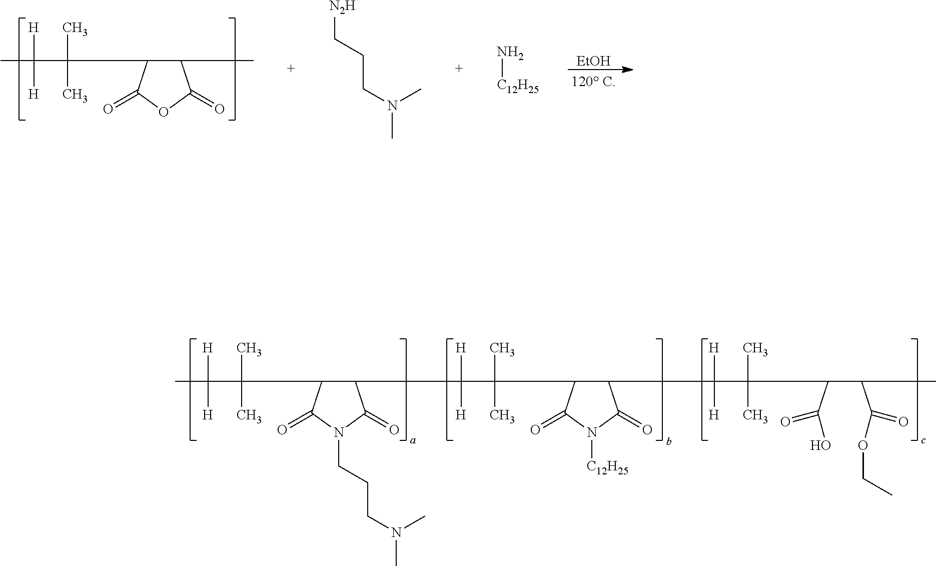

A quantity of 9.36 g of poly(IB-co-MA) (M_{w} 80,000 Da), 3.72 g of DMAPA, 1.23 g triethylamine, 2.25 g of dodecylamine and 30.77 g ethanol were charged into a sealed stainless steel reactor. The mixture was heated at 120° C. for 10 hours. The polymer solution was then cooled and discharged. The ethanol and triethylamine were removed by vacuum stripping and solvent exchange with water. Then, it was neutralized by 1 M HCl solution (1:1 molar of DMAPA). The water was then removed by vacuum stripping and a white powder resulted.

### Example 42

### Grafting of Poly(IB-Co-MA) (M_{w} 240,000 Da) with N-(3-Dimethylaminopropyl)Amine, Ethanol, and Dodecylamine (60% Dimethylaminopropyl Imide, 20% Dodecylimide, 20% Ethyl Ester; Molar Ratios)

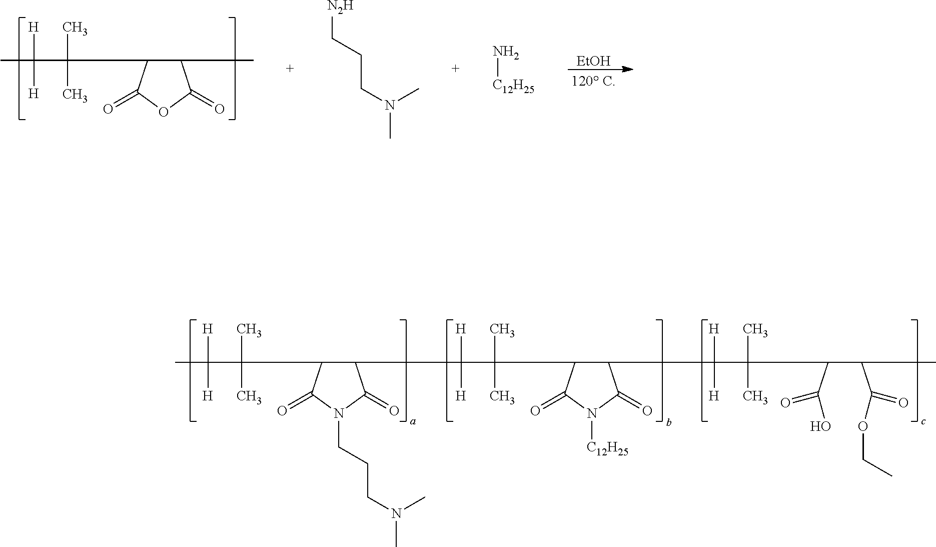

A quantity of 9.36 g of poly(IB-co-MA) (M_{w} 240,000 Da), 3.72 g of DMAPA, 1.23 g triethylamine, 2.25 g of dodecylamine and 30.77 g ethanol were charged into a sealed stainless steel reactor. The mixture was heated at 120° C. for 10 hours. The polymer solution was then cooled and discharged. The ethanol and triethylamine were removed by vacuum stripping and solvent exchange with water. Then, it was neutralized by 1 M HCl solution (1:1 molar of DMAPA). The water was then removed by vacuum stripping and a white powder resulted.

### Example 43

### Grafting of Poly(IB-Co-MA) (M_{w} 80,000 Da) with N-(3-Dimethylaminopropyl)Amine, Butylamine, Propylene Oxide/Ethylene Oxide Amine, Ethanol (60% Dimethylaminopropyl Imide, 20% Butylimide, 0.01% Propylene Oxide/Ethylene Oxide Imide, 19% Ethyl Ester; Molar Ratios)

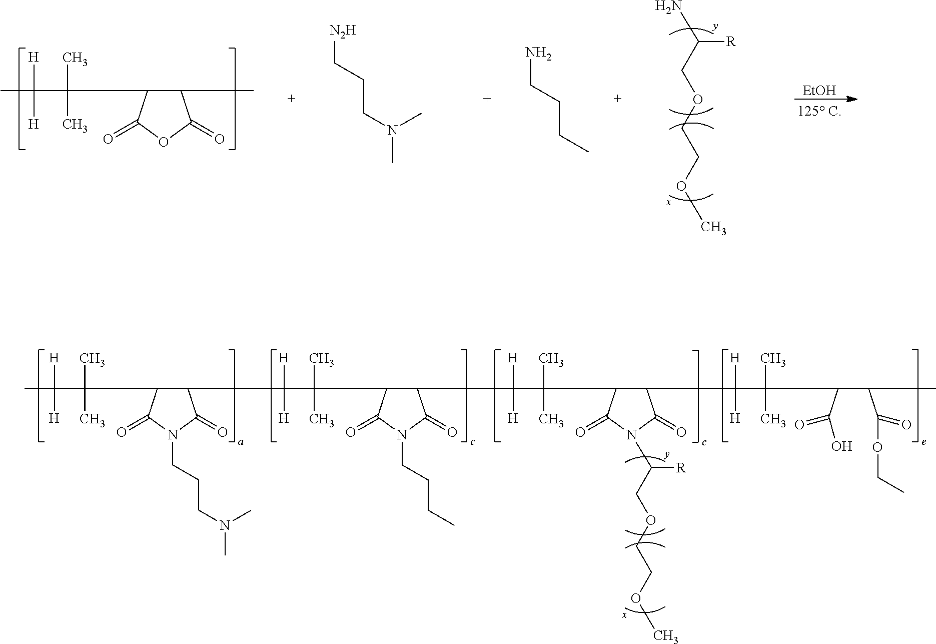

A quantity of 117.73 g of poly(IB-co-MA) (M_{w} 80,000 Da), 46.81 g DMAPA, 11.17 g butylamine, 14.68 g triethylamine, 16.74 g Jeffamine M2070 (a polyethylene/polypropylene amine, Huntsman Corporation) and 384.64 g ethanol were added to a sealed 1 L stainless steel reactor. The mixture was heated to 120° C. and allowed to react for 10 hours. The polymer solution was then cooled and discharged from the reactor. Ethanol and triethylamine were removed by vacuum stripping and the solution was solvent exchanged with water. The solution was then neutralized with 1 M HCl (1:1 molar of DMAPA). Water was then removed by vacuum stripping, resulting in a white powder.

### Example 44

### Antimicrobial Activity by Plate Streak Method

The antimicrobial activity of the various polymers was evaluated using a plate streak method. The antimicrobial activity was screened against various microorganism including Staphylococcus aureus (ATCC 65380), Escherichia coli (ATCC 8730), Pseudomonas aeruginosa (ATCC 9027), Candida albicans (ATCC 10231) and Aspergillus niger (ATCC 16404). Briefly, a stock solution of each microorganism was prepared by growing the bacterial cells in tryptic soy broth (TSB) or the fungi cells in yeast malt broth (YM) to reach a concentration of about 10⁸-10⁹ cfu/mL. Molten agar (TSA or YM) was seeded with each microorganism to obtain a microbial concentration of about 10⁵-10⁶ cfu/mL. Plates were allowed to solidify. The polymers were tested either as a 5% solution in water or as powders by streaking the solution or sprinkling the polymers over the microbial seeded plate, respectively. The plates were refrigerated for 24 hours to allow for the polymer to diffuse and were then placed in the incubator (32° C. for bacteria plates, 28° C. for fungal plates) for 24-72 hours. Growth inhibition along the polymeric streak or polymer sprinkles was considered as indicative of antimicrobial activity. Seeded plates without polymers were used as positive controls for microbial growth. The results of the streak test for various polymers are summarized in Table 1. A “−” symbol indicates that antimicrobial activity was observed (growth inhibition) whereas a “+” symbol indicates that no antimicrobial activity was detected in this assay. The letters NT means “not tested.”

TABLE 1
Bacteria and fungi growth inhibition test results of Example 44.

| | growth inhibition | | | | |
|---|---|---|---|---|---|
| | S. | E. | P. | A. | C. |
| polymer | aureus | coli | aeruginosa | niger | albicans |
| Example 1 | − | − | − | + | + |
| Example 2 | + | + | + | + | + |
| Example 3 | − | − | − | + | + |
| Example 4 | − | − | + | + | − |
| Example 5 | − | − | − | + | + |
| Example 6 | − | + | + | + | + |
| Example 7 | − | − | − | + | − |
| Example 8 | − | − | − | + | − |
| Example 9 | − | − | − | + | − |
| Example 10 | + | + | + | + | + |
| Example 11 | − | − | + | + | + |
| Example 12 | + | + | + | + | + |
| Example 13 | + | + | + | + | + |
| Example 14 | + | + | + | + | + |
| Example 15 | − | + | + | + | + |
| Example 16 | − | + | + | + | + |
| Example 17 | + | + | + | + | + |
| Example 18 | + | + | + | + | + |
| Example 19 | + | + | + | + | + |
| Example 20 | + | + | + | + | + |
| Example 21 | + | + | + | + | + |
| Example 22 | + | + | + | NT | NT |
| Example 23 | + | + | + | NT | NT |
| Example 24 | + | + | + | NT | NT |
| Example 25 | + | − | − | NT | NT |
| Example 26 | + | + | + | NT | NT |
| Example 27 | + | + | + | NT | NT |
| Example 28 | + | + | + | NT | NT |
| Example 29 | + | + | + | NT | NT |
| Example 30 | + | + | + | NT | NT |
| Example 31 | + | − | − | NT | NT |
| Example 32 | + | + | + | NT | NT |
| Example 33 | + | + | + | NT | NT |
| Example 34 | + | + | + | NT | NT |
| Example 35 | + | + | + | NT | NT |
| Example 36 | + | + | + | NT | NT |
| Example 37 | − | − | + | + | + |
| Example 38 | − | − | + | + | + |
| Example 39 | + | + | + | + | + |
| Example 40 | − | + | + | + | + |
| Example 41 | − | + | + | + | + |
| Example 43 | − | − | + | + | + |

As shown in Table 1, polymers embraced by this invention exhibit antimicrobial activity.

### Example 45

### Antimicrobial Activity by Shake Flask Method

The antimicrobial activity of selected polymers was further evaluated by a shake flask method. Briefly, 2% by wt. of the polymers were added to TSB. The pH of the media was adjusted to a pH of about 6. Then, each flask was inoculated with a microorganism to achieve an initial concentration of about 10⁶ cfu/mL and incubated with shaking at 32° C. Microbial counts were conducted after 48 hours by serially diluting and plating onto TSA media. Test results are summarized in Table 2. The values indicate log reduction (Log CFU/mL control at t=48 h−Log CFU/mL treated sample at t=48 h) of each polymer tested against S. aureus, E. coli and P. aeruginosa.

TABLE 2
Microbe log reductions for Example 45

| | log reduction | | |
|---|---|---|---|
| polymer | S. aureus | E. coli | P. aeruginosa |
| Example 5 | >7.2 | >7.2 | 6.0 |
| Example 7 | >7.2 | >7.2 | 7.0 |

As shown in Table 2, the antimicrobial activity of Example 5 and example 7 when tested at 2% resulted in total growth inhibition of both S. aureus and E. coli. Example 7 further reduced the counts of P. aeruginosa by 7 logs, whereas Example 5 provided a 6-log reduction in the P. aeruginosa counts.

### Example 45

### Minimum Inhibitory Concentration Testing

Additional microbiology testing was performed to determine minimal inhibitory concentrations (MIC) and cidal concentrations against various microbes, including Bacteria cepacia (ATCC 25416), Staphylococcus aureus (ATCC 65380), Escherichia coli (ATCC 8730), Pseudomonas aeruginosa (ATCC 9027), Candida albicans (ATCC 10231) and Aspergillus niger (ATCC 16404) (Tables 3 and 4). Two molecular weight poly(IB-co-MA) polymers were studied, 6,000 Da and 15,000 Da. In addition to protonation by HCl, two functionalized polymers were protonated using organic acids.

The evaluated polymers showed antimicrobial activity against E. coli, S. aureus, and P. aeruginosa.

TABLE 3
MIC results for Example 45

| | minimum inhibitory concentration (ppm) | | | |
|---|---|---|---|---|
| polymer | E. coli | P. aeruginosa | B. cepacia | S. aureus |
| p(IB-co-MA), M_{w} = 6,000 Da | <10 | 2,500 | >20,000 | 1,250 |
| 60% dimethylaminopropylimide/20% | | | | |
| butyl imide/20% ethyl ester | | | | |
| 12266-18 | | | | |
| p(IB-co-MA), M_{w} = 15,000 Da | <10 | 2,500 | >20,000 | 2,500 |
| 60% dimethylaminopropylimide/20% | | | | |
| butyl imide/20% ethyl ester | | | | |
| 12266-62 | | | | |
| p(IB-co-MA), M_{w} = 6,000 Da | 600 | >10,000 | >10,000 | 1,250 |
| 60% dimethylaminopropylimide/20% | | | | |
| butyl imide/20% ethyl ester, | | | | |
| protonation with HCl | | | | |
| 12368-3a | | | | |
| p(IB-co-MA), M_{w} = 6,000 Da | 150 | 10,000 | >10,000 | 1,250 |
| 60% dimethylaminopropylimide/20% | | | | |
| butyl imide/20% ethyl ester, | | | | |
| protonated with sorbic acid | | | | |
| 12368-3b | | | | |
| p(IB-co-MA), M_{w} = 6,000 Da | 300 | >10,000 | >10,000 | 1,250 |
| 60% dimethylaminopropylimide/20% | | | | |
| butyl imide/20% ethyl ester, | | | | |
| protonated with lactic acid | | | | |
| 12368-3c | | | | |

TABLE 4
Cidal concentration results for Example 45

| | cidal concentration (ppm) | | | |
|---|---|---|---|---|
| polymer | E. coli | P. aeruginosa | B. cepacia | S. aureus |
| p(IB-co-MA), M_{w} = 6,000 Da | 600 | 5,000 | >20,000 | 1,250 |
| 60% dimethylaminopropylimide/20% | | | | |
| butyl imide/20% ethyl ester | | | | |
| p(IB-co-MA), M_{w} = 15,000 Da | 75 | 2,500 | >20,000 | 5,000 |
| 60% dimethylaminopropylimide/20% | | | | |
| butyl imide/20% ethyl ester | | | | |
| p(IB-co-MA), M_{w} = 6,000 Da | 600 | >10,000 | >10,000 | 10,000 |
| 60% dimethylaminopropylimide/20% | | | | |
| butyl imide/20% ethyl ester, | | | | |
| protonation with HCl | | | | |
| p(IB-co-MA), M_{w} = 6,000 Da | 1,250 | >10,000 | >10,000 | 10,000 |
| 60% dimethylaminopropylimide/20% | | | | |
| butyl imide/20% ethyl ester, | | | | |
| protonated with sorbic acid | | | | |
| p(IB-co-MA), M_{w} = 6,000 Da | 600 | >10,000 | >10,000 | 5,000 |
| 60% dimethylaminopropylimide/20% | | | | |
| butyl imide/20% ethyl ester, | | | | |
| protonated with lactic acid | | | | |

### Example 45

### Antimicrobial Performance in Skin Creams

Two microbial tests were conducted to determine the efficacy of the multifunctional polymers in skin creams originally formulated without a preservative. Both skin creams were post-formulated by blending with 1% (w/w) of the polymer from Example 3. These two formulas and their controls (which did not contain multifunctional polymer) were challenged against Bacillus licheniformis (ATCC 27326), Bacillus megaterium (ATCC 27327), Bacillus subtilis (ATCC 27348), Enterobacter cloacae (ATCC 13047), Pseudomonas aeruginosa (ATCC 10145), and a mixture of the five microbes (Table 5).

The first test was a direct streak onto tryptic soy agar (TSA, for bacteria), yeast malt extract agar (YM, for yeast and fungi), and potato dextrose agar (PDA, adjusted to pH 3.5 for fungi and yeast) plates. The plates were incubated for 24-48 hours at 32° C. for the detection of bacteria, and 3-7 days at 28° C. for fungal and yeast contaminants. No microbial growth along the streak was observed in the samples, indicating that the corresponding sample did not contain viable microbial cells.

For the second test in-can preservation was performed in accordance with the ASTM D2574-94, “Resistance of Emulsion Paints in the Container to Attack of Microorganisms”. Briefly, each sample was inoculated with individual broth cultures containing each of the test bacteria. The legend for in-can testing is summarized in Table 6, and the final bacterial concentration levels recorded for each bacteria are indicated in Tables 7-12. Samples were also inoculated with a mixed broth culture containing all five bacteria. The inoculated samples were mixed vigorously and incubated at 32° C. for the duration of the test. At appropriate intervals, the samples were checked for the presence of viable microorganisms by directly streaking the sample onto TSA plates with a sterile cotton swab. The plates were incubated for 48 hours at 32° C. The plates were then rated on a scale of “0” to “4” based upon the number of colony forming units observed.

All of the samples containing the multifunctional polymer were adequately protected against the test microorganisms individually and when combined. All unprotected controls, except the sample inoculated with B. licheniformis, were susceptible to microbial spoilage. The B. licheniformis unprotected control was protected by the final day of the second microbial challenge.

TABLE 5
Challenge concentrations for Example 45.

microbe concentration (cfu/mL)

| microbe | challenge I | challenge II |
|---|---|---|
| Bacillus licheniformis | 1.45 × 10⁶ | 1.36 × 10⁷ |
| Bacillus megaterium | 1.42 × 10⁶ | 1.48 × 10⁷ |
| Bacillus subtilis | 1.41 × 10⁶ | 1.45 × 10⁷ |
| Enterobacter cloacae | 1.91 × 10⁶ | 1.87 × 10⁷ |
| Pseudomonas aeruginosa | 1.84 × 10⁶ | 1.87 × 10⁷ |
| Mixed Culture | 1.88 × 10⁶ | 1.81 × 10⁷ |

TABLE 6
Legend for in-can preservation testing

| rating | meaning |
|---|---|
| — | no growth |
| 1 | trace growth (1-9 colonies per “streak-inch”). |
| 2 | light growth (10-99 colonies per “streak-inch”). |
| 3 | moderate growth (greater than 100 colonies, but |
| | still distinguishable). |
| 4 | heavy growth (continuous smear of growth). |

TABLE 7
B. licheniformis results

| | challenge I | | | | challenge II | | | |
|---|---|---|---|---|---|---|---|---|
| | days | | | | days | | | |
| sample | 1 | 2 | 3 | 6 | 1 | 2 | 3 | 6 |
| skin cream 1, unprotected control | 0 | 0 | 0 | 0 | 1 | 1 | 1 | 1 |
| skin cream 1 + multfunctional | 0 | 0 | 0 | 0 | 0 | 0 | 0 | 0 |
| polymer | | | | | | | | |
| skin cream 2, unprotected control | 2 | 1 | 1 | 0 | 4 | 2 | 1 | 0 |
| skin cream 2 + multfunctional | 0 | 0 | 0 | 0 | 0 | 0 | 0 | 0 |
| polymer | | | | | | | | |

TABLE 8
B. megaterium results

| | challenge I | | | | challenge II | | | |
|---|---|---|---|---|---|---|---|---|
| | days | | | | days | | | |
| sample | 1 | 2 | 3 | 6 | 1 | 2 | 3 | 6 |
| skin cream 1, unprotected control | 0 | 0 | 0 | 0 | 2 | 1 | 1 | 1 |
| skin cream 1 + multfunctional | 0 | 0 | 0 | 0 | 0 | 0 | 0 | 0 |
| polymer | | | | | | | | |
| skin cream 2, unprotected control | 1 | 1 | 1 | 1 | 2 | 1 | 1 | 1 |
| skin cream 2 + multfunctional | 0 | 0 | 0 | 0 | 0 | 0 | 0 | 0 |
| polymer | | | | | | | | |

TABLE 9
B. subtilis results

| | challenge I | | | | challenge II | | | |
|---|---|---|---|---|---|---|---|---|
| | days | | | | days | | | |
| sample | 1 | 2 | 3 | 6 | 1 | 2 | 3 | 6 |
| skin cream 1, unprotected control | 2 | 2 | 0 | 0 | 2 | 2 | 1 | 1 |
| skin cream 1 + multfunctional | 0 | 0 | 0 | 0 | 0 | 0 | 0 | 0 |
| polymer | | | | | | | | |
| skin cream 2, unprotected control | 2 | 2 | 0 | 0 | 3 | 2 | 2 | 2 |
| skin cream 2 + multfunctional | 0 | 0 | 0 | 0 | 0 | 0 | 0 | 0 |
| polymer | | | | | | | | |

TABLE 10
E. cloacae results

| | challenge I | | | | challenge II | | | |
|---|---|---|---|---|---|---|---|---|
| | days | | | | days | | | |
| sample | 1 | 2 | 3 | 6 | 1 | 2 | 3 | 6 |
| skin cream 1, unprotected control | 4 | 4 | 4 | 4 | 4 | 4 | 4 | 4 |
| skin cream 1 + multfunctional | 2 | 2 | 1 | 0 | 4 | 4 | 3 | 0 |
| polymer | | | | | | | | |
| skin cream 2, unprotected control | 4 | 4 | 4 | 4 | 4 | 4 | 4 | 4 |
| skin cream 2 + multfunctional | 0 | 0 | 0 | 0 | 0 | 0 | 0 | 0 |
| polymer | | | | | | | | |

TABLE 11
P. aeruginosa results

| | challenge I | | | | challenge II | | | |
|---|---|---|---|---|---|---|---|---|
| | days | | | | days | | | |
| sample | 1 | 2 | 3 | 6 | 1 | 2 | 3 | 6 |
| skin cream 1, unprotected control | 2 | 1 | 1 | 0 | 4 | 4 | 4 | 3 |
| skin cream 1 + multfunctional | 2 | 2 | 1 | 0 | 2 | 2 | 2 | 0 |
| polymer | | | | | | | | |
| skin cream 2, unprotected control | 4 | 4 | 3 | 1 | 4 | 4 | 4 | 4 |
| skin cream 2 + multfunctional | 0 | 0 | 0 | 0 | 0 | 0 | 0 | 0 |
| polymer | | | | | | | | |

TABLE 12
mixed microbes results

| | challenge I | | | | challenge II | | | |
|---|---|---|---|---|---|---|---|---|
| | days | | | | days | | | |
| sample | 1 | 2 | 3 | 6 | 1 | 2 | 3 | 6 |
| skin cream 1, unprotected control | 4 | 3 | 3 | 3 | 4 | 4 | 4 | 4 |
| skin cream 1 + multfunctional | 2 | 2 | 1 | 0 | 4 | 4 | 3 | 0 |
| polymer | | | | | | | | |
| skin cream 2, unprotected control | 4 | 4 | 4 | 4 | 4 | 4 | 4 | 4 |
| skin cream 2 + multfunctional | 0 | 0 | 0 | 0 | 0 | 0 | 0 | 0 |
| polymer | | | | | | | | |

While a number of embodiments of this invention have been represented, it was apparent that the basic construction can be altered to provide other embodiments that utilize the invention without departing from the spirit and scope of the invention. All such modifications and variations are intended to be included within the scope of the invention as defined in the appended claims rather than the specific embodiments that have been presented by way of example.

We claim:

1. A polymer comprising at least:
(A) at least a first repeating unit selected from the group consisting of: 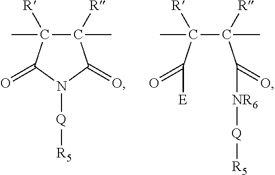
and combinations thereof, and
(B) at least a second repeating unit selected from the group consisting of: 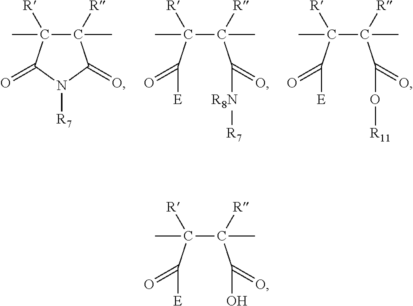
and combinations thereof,
wherein
each C— indicates a bond from said unit to another unit along the polymer backbone;
each R′ and R″ is independently selected from the group consisting of: hydrogen, alkyl, cycloalkyl, aryl, and combinations thereof, wherein said R′ and R″ may optionally form a ring;
each R₅ is independently selected from the group consisting of —NR₉R₁₀, functionalized and unfunctionalized nitrogen or phosphorus containing C₅-C₇ cyclic groups, and mixtures thereof;
each R₆, R₈, R₉, and R₁₀ is independently selected from the group consisting of hydrogen, functionalized and unfunctionalized alkyl, alkoxy, cycloalkyl, alkenyl, cycloalkenyl, aryl, wherein any of the before mentioned groups may be with or without heteroatoms, and mixtures thereof;
each R₇ and R₁₁ is independently selected from the group consisting of functionalized and unfunctionalized alkyl, alkoxy, cycloalkyl, alkenyl, cycloalkenyl, and aryl, wherein any of the before mentioned groups may be with or without heteroatoms, and mixtures thereof;
each Q is independently selected from the group consisting of functionalized or unfunctionalized alkylene, cycloalkylene, and combinations thereof, wherein any of the functionalized or unfunctionalized alkylene may be with or without heteroatoms, and mixtures thereof;
each E is independently selected from the group consisting of —OM, —OR₇, —NHR₇, —NR₇R₁₁, and mixtures thereof; and
each M is independently selected from the group consisting of hydrogen, alkali metal ions, alkaline earth metal ions, ammonium ions, and mixtures thereof, and
wherein said polymer has a weight-average molecular weight less than 50,000 Da.

2. The polymer according to claim 1 wherein each R′ and R″ is hydrogen.

3. The polymer according to claim 1, wherein each R₅ is independently —NR₉R₁₀ or a di-(C₁-C₈ alkyl)amino group or imidazolyl group, wherein said R₉ and R₁₀.

4. The polymer according to claim 1, wherein each Q is independently functionalized or unfunctionalized C₁-C₄ alkylene.

5. The polymer according to claim 1, wherein each R₇ is independently functionalized or unfunctionalized C₁-C₉ alkyl.

6. The polymer according to claim 1, wherein each R₁₁ is independently functionalized or unfunctionalized C₁-C₇ alkyl.

7. The polymer according to claim 1 that comprises at least another repeating unit which is the polymerized residue of a monomer selected from the group consisting of: alpha-olefins, vinyl ethers, styrenes, (meth)acrylates, (meth)acrylamides, 4-vinyl-1,2,3-triazoles, 5-vinyl-1,2,3-triazoles, vinyls, allyls, maleates, maleimides, α-β-olefinically unsaturated carboxylic nitriles, vinyl esters, vinyl acetates, vinyl amides, vinyl alcohols, vinyl carbonates, vinyl carbamates, vinyl thiocarbamates, vinyl ureas, vinyl halides, vinyl imidazoles, vinyl lactams, vinyl pyridines, vinyl silanes, vinyl siloxanes, vinyl sulfones, allyl ethers, and combinations thereof.

8. The polymer according to claim 7 wherein said polymer has the structure: 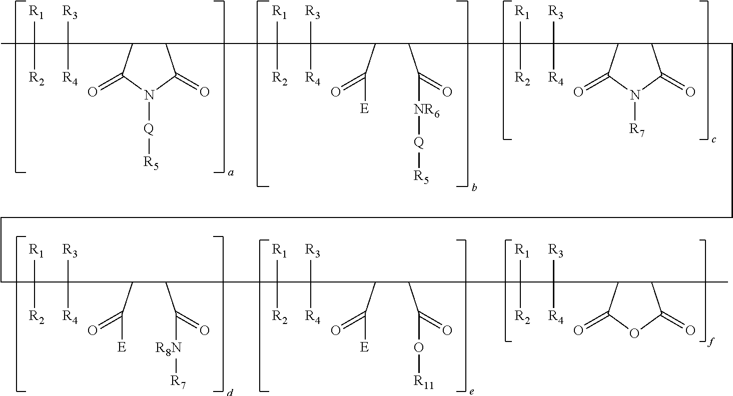
wherein
each R₁, R₂, R₃, R₄, R₆, R₈, R₉, and R₁₀ is independently selected from the group consisting of hydrogen, functionalized and unfunctionalized alkyl, alkoxy, cycloalkyl, alkenyl, cycloalkenyl, aryl, wherein any of the before mentioned groups may be with or without heteroatoms, and mixtures thereof;
each R₅ is independently selected from the group consisting of —NR₉R₁₀, functionalized and unfunctionalized nitrogen or phosphorus containing C₅-C₇ cyclic groups, and mixtures thereof;
each R₇ and R₁₁ is independently selected from the group consisting of functionalized and unfunctionalized alkyl, alkoxy, cycloalkyl, alkenyl, cycloalkenyl, and aryl, wherein any of the before mentioned groups may be with or without heteroatoms, and mixtures thereof;
each Q is independently selected from the group consisting of functionalized or unfunctionalized alkylene, cycloalkylene, and combinations thereof, wherein any of the functionalized or unfunctionalized alkylene may be with or without heteroatoms, and mixtures thereof;
each E is independently selected from the group consisting of —OM, —OR₇, —NHR₇, —NR₇R₁₁, and mixtures thereof;
each M is independently selected from the group consisting of hydrogen, alkali metal ions, alkaline earth metal ions, ammonium ions, and mixtures thereof; and
a, b, c, d, e, and f are mole percents whose sum in each polymer equals 100%, where at least one of a and b is not zero; and at least one of c, d, and e is not zero, wherein said polymer is alternating, block, or random.

9. The polymer according to claim 7 wherein said another repeating unit which is the polymerized residue of a monomer is selected from the group consisting of: ethylene, propylene, 1-butene, 2-butene, iso-butylene, 1-decene, methyl vinyl ether, ethyl vinyl ether, iso-propyl vinyl ether, n-butyl vinyl ether, iso-butyl vinyl ether, sec-butyl vinyl ether, octyl vinyl ether, decyl vinyl ether, dodecyl vinyl ether, hexadecyl vinyl ether, octadecyl vinyl ether, styrene, α-methylstyrene and combinations thereof.

10. The polymer according to claim 9 having a structure selected from the group consisting of: 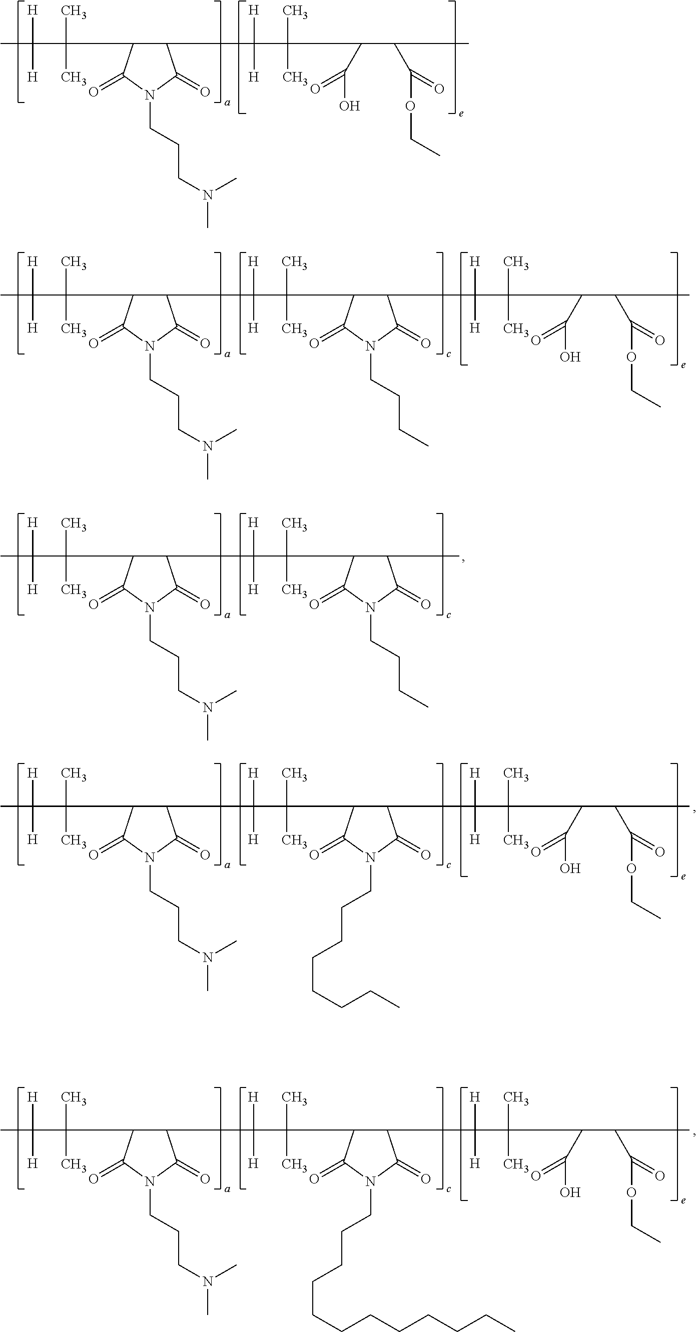 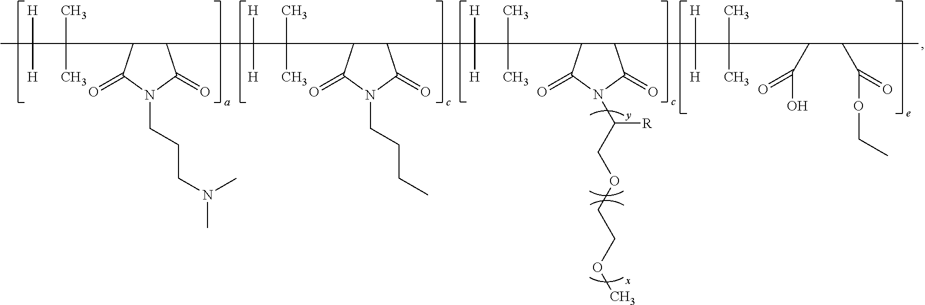 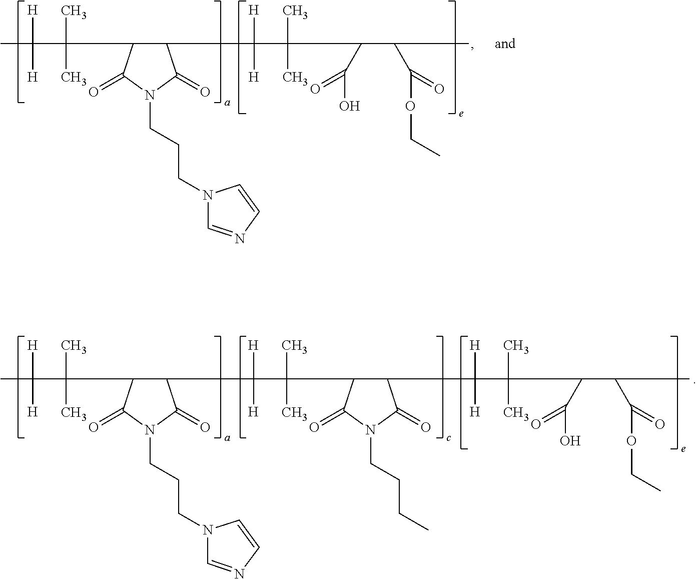
wherein said subscripts a, b, c, d, and e are molar ratios whose sum in each polymer equals 100%.

11. The polymer according to claim 1, wherein said polymer exhibits antimicrobial activity against a microbe selected from the group consisting of S. aureus, E. coli, P. aeruginosa, A. niger, C. albicans, and mixtures thereof.

12. A composition having at least one polymer comprising:
(A) at least a first repeating unit selected from the group consisting of: 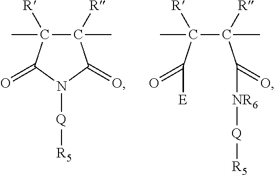
and combinations thereof, and
(B) at least a second repeating unit is selected from the group consisting of: 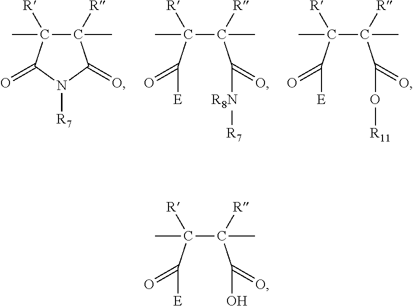
and combinations thereof,
wherein
each C— indicates a bond from said unit to another unit along the polymer backbone;
each R′ and R″ is independently selected from the group consisting of: hydrogen, alkyl, cycloalkyl, aryl, and combinations thereof;
each R₅ is independently selected from the group consisting of —NR₉R₁₀, functionalized and unfunctionalized nitrogen or phosphorus containing C₅-C₇ cyclic groups, and mixtures thereof;
each R₆, R₈, R₉, and R₁₀ is independently selected from the group consisting of hydrogen, functionalized and unfunctionalized alkyl, alkoxy, cycloalkyl, alkenyl, cycloalkenyl, aryl, wherein any of the before mentioned groups may be with or without heteroatoms, and mixtures thereof;
each R₇ and R₁₁ is independently selected from the group consisting of functionalized and unfunctionalized alkyl, alkoxy, cycloalkyl, alkenyl, cycloalkenyl, and aryl, wherein any of the before mentioned groups may be with or without heteroatoms, and mixtures thereof;
each Q is independently selected from the group consisting of functionalized or unfunctionalized alkylene, cycloalkylene, and combinations thereof, wherein any of the functionalized or unfunctionalized alkylene may be with or without heteroatoms, and mixtures thereof;
each E is independently selected from the group consisting of —OM, —OR₇, —NHR₇, —NR₇R₁₁, and mixtures thereof;
each M is independently selected from the group consisting of hydrogen, alkali metal ions, alkaline earth metal ions, ammonium ions, and mixtures thereof, and
wherein said polymer has a weight-average molecular weight of less than 50,000 Da.

13. The composition according to claim 12 wherein said R′ and R″ are hydrogen.

14. The composition according to claim 12 wherein said polymer comprises at least another repeating unit which is the polymerized residue of a monomer selected from the group consisting of: alpha-olefins, vinyl ethers, styrenes, (meth)acrylates, (meth)acrylamides, styrenes, 4-vinyl-1,2,3-triazoles, 5-vinyl-1,2,3-triazoles, vinyls, allyls, maleates, maleimides, α-β-olefinically unsaturated carboxylic nitriles, vinyl esters, vinyl acetates, vinyl amides, vinyl alcohols, vinyl carbonates, vinyl carbamates, vinyl thiocarbamates, vinyl ureas, vinyl halides, vinyl imidazoles, vinyl lactams, vinyl pyridines, vinyl silanes, vinyl siloxanes, vinyl sulfones, allyl ethers, and combinations thereof.

15. The composition according to claim 14 wherein said another repeating unit which is the polymerized residue of a monomer is selected from the group consisting of: ethylene, propylene, 1-butene, 2-butene, iso-butylene, 1-decene, methyl vinyl ether, ethyl vinyl ether, iso-propyl vinyl ether, n-butyl vinyl ether, iso-butyl vinyl ether, sec-butyl vinyl ether, octyl vinyl ether, decyl vinyl ether, dodecyl vinyl ether, hexadecyl vinyl ether, octadecyl vinyl ether, styrene, α-methylstyrene and combinations thereof.

16. The composition according to claim 15 wherein said polymer has the structure: 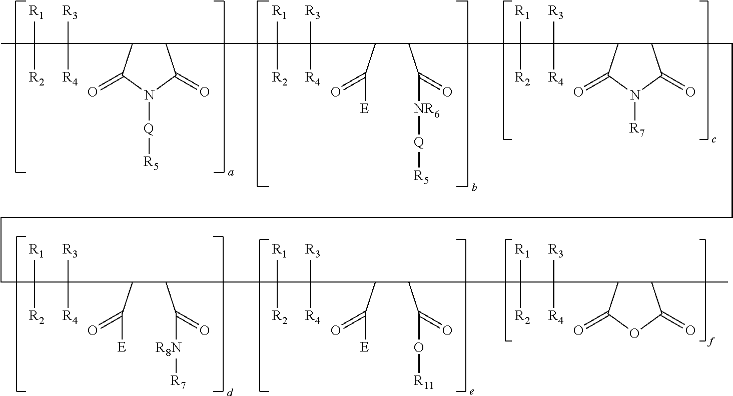
wherein each R₁, R₂, R₃, R₄, R₆, R₈, R₉, and R₁₀ is independently selected from the group consisting of hydrogen, functionalized and unfunctionalized alkyl, alkoxy, cycloalkyl, alkenyl, cycloalkenyl, aryl, wherein any of the before mentioned groups may be with or without heteroatoms, and mixtures thereof;
each R₅ is independently selected from the group consisting of —NR₉R₁₀, functionalized and unfunctionalized nitrogen or phosphorus containing C₅-C₇ cyclic groups, and mixtures thereof;
each R₇ and R₁₁ is independently selected from the group consisting of functionalized and unfunctionalized alkyl, alkoxy, cycloalkyl, alkenyl, cycloalkenyl, and aryl, wherein any of the before mentioned groups may be with or without heteroatoms, and mixtures thereof;
each Q is independently selected from the group consisting of functionalized or unfunctionalized alkylene, cycloalkylene, and combinations thereof, wherein any of the functionalized or unfunctionalized alkylene may be with or without heteroatoms, and mixtures thereof;
each E is independently selected from the group consisting of —OM, —OR₇, —NHR₇, —NR₇R₁₁, and mixtures thereof;
each M is independently selected from the group consisting of hydrogen, alkali metal ions, alkaline earth metal ions, ammonium ions, and mixtures thereof; and
a, b, c, d, e, and f are mole percents whose sum in each polymer equals 100%, with the proviso that at least one of a and b is not zero; and at least one of c, d, and e is not zero, wherein said polymer is alternating, block, or random.

17. The composition according to claim 16, wherein said polymer has a structure selected from the group consisting of: 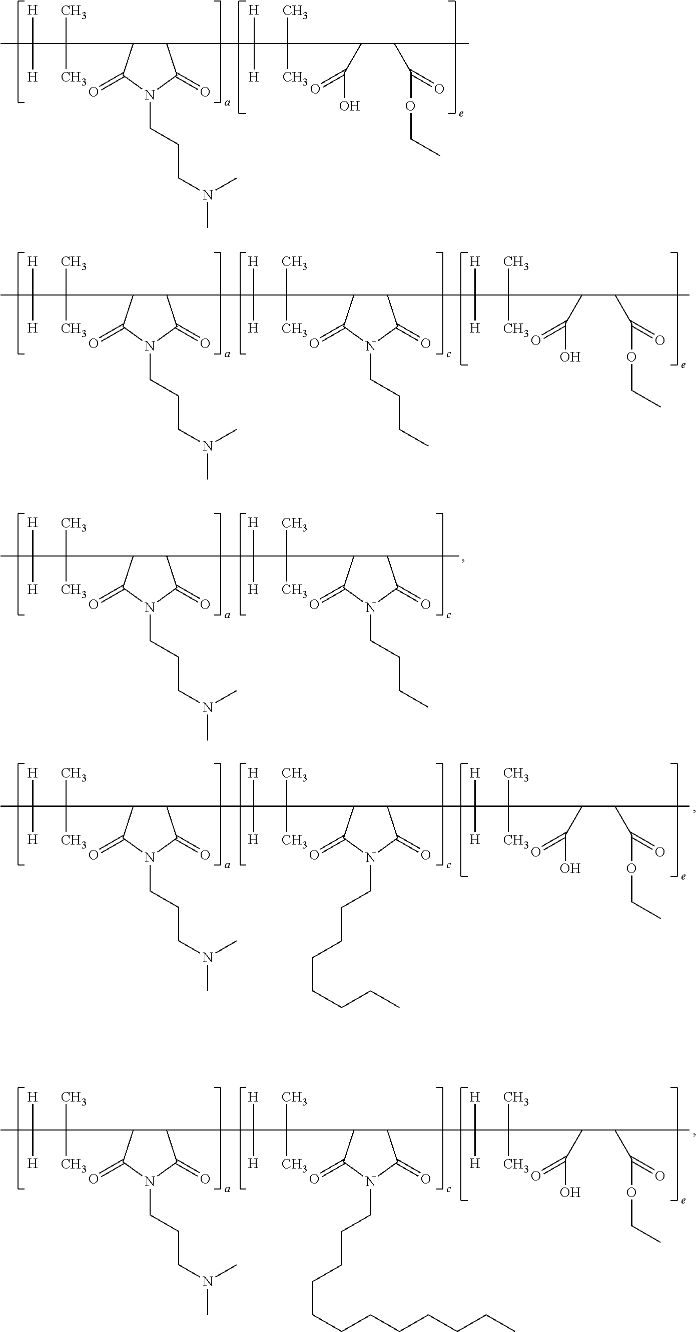 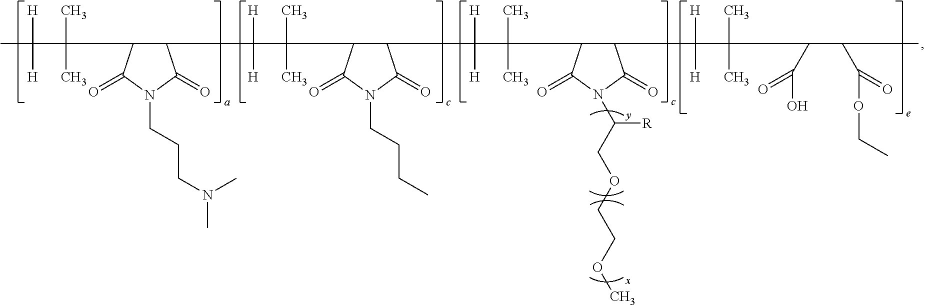 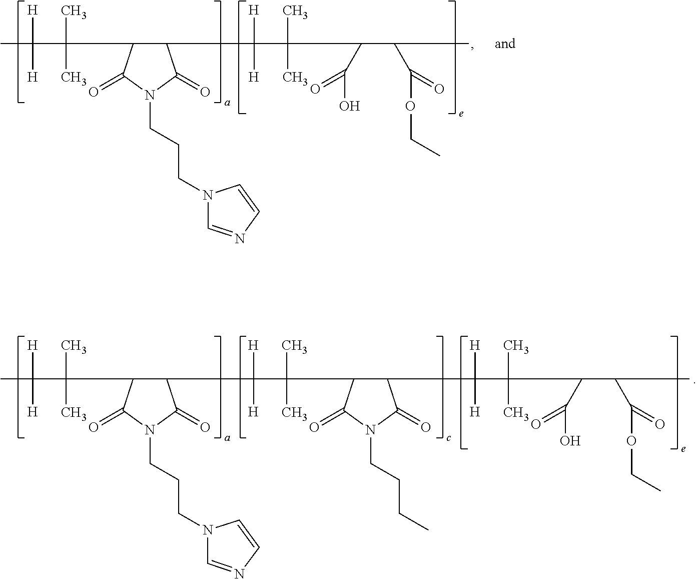
wherein said subscripts a, b, c, d, and e are molar ratios whose sum in each polymer equal 100%.

18. The composition according to claim 12, wherein said polymer exhibits antimicrobial activity against a microbe selected from the group consisting of S. aureus, E. coli, P. aeruginosa, A. niger, C. albicans, and mixtures thereof.

19. The composition according to claim 12 that is a nutrition, food, beverage, pharmaceutical, cleaning, coating, biocide, construction, energy, industrial, oilfield, personal care, household, performance, agricultural, cosmetic, pesticide, veterinary, fuel, lubricant, adhesive, electronic, textile, ink, or membrane composition.

20. The composition according to claim 19, wherein said personal care composition is a color cosmetic, hair care, skin care, sun care, or oral care composition.

21. A method of providing antimicrobial activity, said method comprising the step: contacting a compound or a composition with at least one polymer comprising:
(A) at least a first repeating unit selected from the group consisting of: 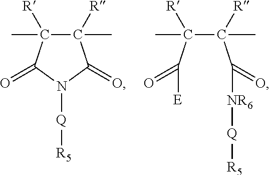
and combinations thereof, and
(B) at least a second repeating unit is selected from the group consisting of: 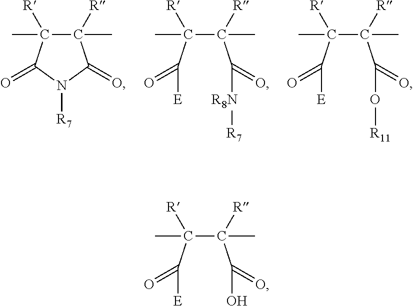
and combinations thereof,
wherein
each C— indicates a bond from said unit to another unit along the polymer backbone;
each R′ and R″ is independently selected from the group consisting of: hydrogen, alkyl, cycloalkyl, aryl, and combinations thereof;
each R₅ is independently selected from the group consisting of —NR₉R₁₀, functionalized and unfunctionalized nitrogen or phosphorus containing C₅-C₇ cyclic groups, and mixtures thereof;
each R₆, R₈, R₉, and R₁₀ is independently selected from the group consisting of hydrogen, functionalized and unfunctionalized alkyl, alkoxy, cycloalkyl, alkenyl, cycloalkenyl, aryl, wherein any of the before mentioned groups may be with or without heteroatoms, and mixtures thereof;
each R₇ and R₁₁ is independently selected from the group consisting of functionalized and unfunctionalized alkyl, alkoxy, cycloalkyl, alkenyl, cycloalkenyl, and aryl, wherein any of the before mentioned groups may be with or without heteroatoms, and mixtures thereof;
each Q is independently selected from the group consisting of functionalized or unfunctionalized alkylene, cycloalkylene, and combinations thereof, wherein any of the functionalized or unfunctionalized alkylene may be with or without heteroatoms, and mixtures thereof;
each E is independently selected from the group consisting of —OM, —OR₇, —NHR₇, —NR₇R₁₁, and mixtures thereof;
each M is independently selected from the group consisting of hydrogen, alkali metal ions, alkaline earth metal ions, ammonium ions, and mixtures thereof.

22. The method according to claim 21 wherein said polymer has the structure: 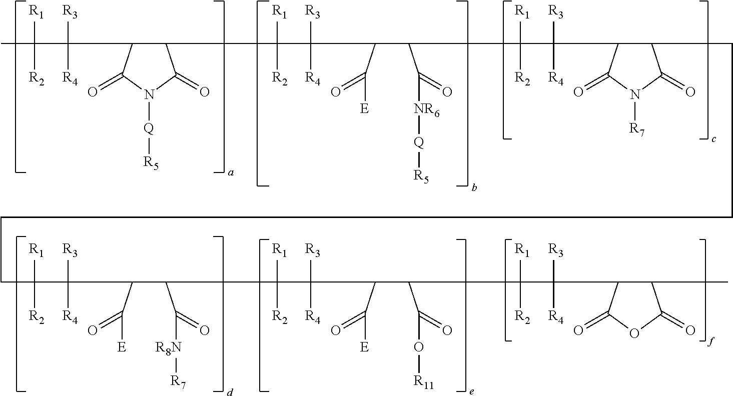
wherein each R₁, R₂, R₃, R₄, R₆, R₈, R₉, and R₁₀ is independently selected from the group consisting of hydrogen, functionalized and unfunctionalized alkyl, alkoxy, cycloalkyl, alkenyl, cycloalkenyl, aryl, wherein any of the before mentioned groups may be with or without heteroatoms, and mixtures thereof;
each R₅ is independently selected from the group consisting of —NR₉R₁₀, functionalized and unfunctionalized nitrogen or phosphorus containing C₅-C₇ cyclic groups, and mixtures thereof;
each R₇ and R₁₁ is independently selected from the group consisting of functionalized and unfunctionalized alkyl, alkoxy, cycloalkyl, alkenyl, cycloalkenyl, and aryl, wherein any of the before mentioned groups may be with or without heteroatoms, and mixtures thereof;
each Q is independently selected from the group consisting of functionalized or unfunctionalized alkylene, cycloalkylene, and combinations thereof, wherein any of the functionalized or unfunctionalized alkylene may be with or without heteroatoms, and mixtures thereof;
each E is independently selected from the group consisting of —OM, —OR₇, —NHR₇, —NR₇R₁₁, and mixtures thereof;
each M is independently selected from the group consisting of hydrogen, alkali metal ions, alkaline earth metal ions, ammonium ions, and mixtures thereof; and
a, b, c, d, e, and f are mole percents whose sum in each polymer equals 100%, with the proviso that at least one of a and b is not zero; and at least one of c, d, and e is not zero, wherein said polymer is alternating, block, or random.

23. The method according to claim 22, wherein said polymer has a structure selected from the group consisting of: 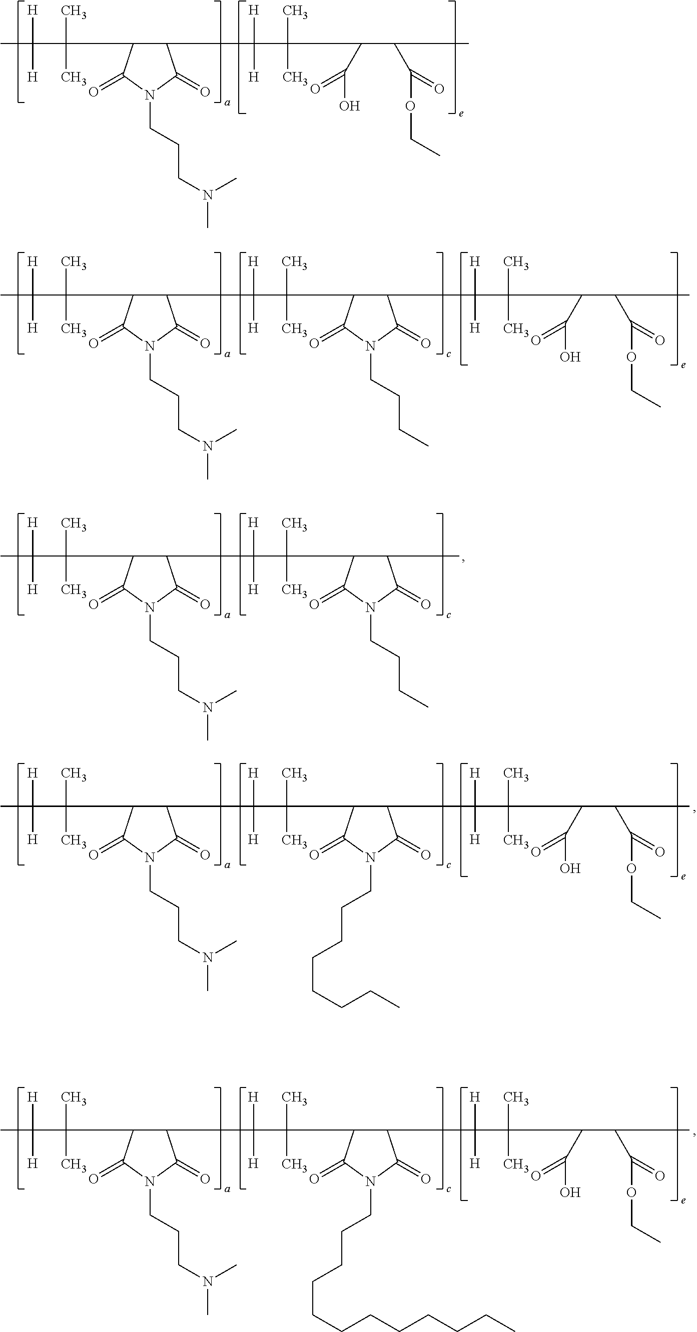 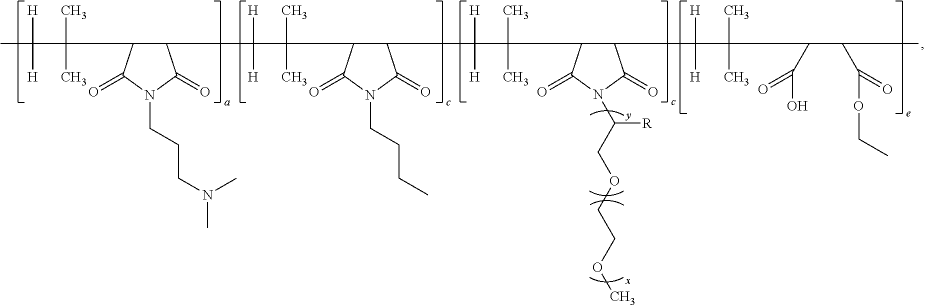 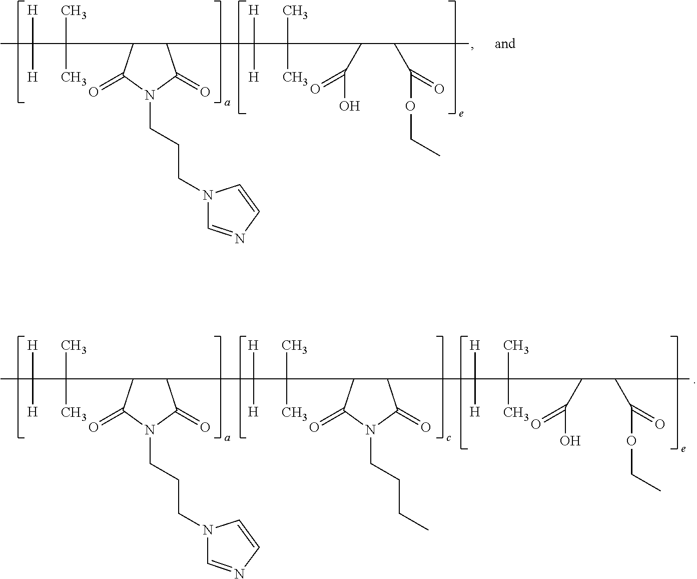
wherein said subscripts a, b, c, d, and e are molar ratios whose sum in each polymer equal 100%.

24. The method according to claim 21 that provides antimicrobial activity against a microbe selected from the group consisting of S. aureus, E. coli, P. aeruginosa, A. niger, C. albicans, and mixtures thereof.
